(19) United States
(12) Patent Application Publication
(10) Pub. No.: US 2024/0325050 A1
(43) Pub. Date: Oct. 3, 2024

(54) INSERTION TOOL WITH A DISSECTOR

(71) Applicant: Senseonics, Incorporated, Germantown, MD (US)

(72) Inventors: Abhi Chavan, Germantown, MD (US); Bryan Hays, Germantown, MD (US); Erman Citirik, Germantow, MD (US)

(73) Assignee: Senseonics, Incorporated, Germantown, MD (US)

(21) Appl. No.: 18/744,083

(22) Filed: Jun. 14, 2024

Related U.S. Application Data
(63) Continuation-in-part of application No. 17/230,610, filed on Apr. 14, 2021.
Application No. 17/230,610 is a continuation-in-part of application No. 16/844,043, filed on Apr. 9, 2020, now Pat. No. 11,432,845.
(60) Provisional application No. 63/010,661, filed on Apr. 15, 2020.
(60) Provisional application No. 62/831,286, filed on Apr. 9, 2019.
(60) Provisional application No. 63/508,143, filed on Jun. 14, 2023.

(51) Int. Cl. A61B 17/34; A61B 17/32

(52) U.S. Cl. CPC A61B 17/3468; A61B 2017/320044; A61B 2017/320056

(57) ABSTRACT

An insertion tool for creating a subcutaneous pocket and implanting a device in the pocket. The tool may include a handle, a tunneling tube, a dissector, and an inserter. The dissector may be configured to create the subcutaneous pocket, move from an extended position to a retracted position, and rotate from an operating position disposed on a longitudinal axis of the tunneling tube to an idle position. The insertor including a cannula and configured to hold the device in a passage of the cannula, rotate from an idle position to an operating position disposed on the longitudinal axis of the tunneling tube, and move from a retracted position to an extended position in which the cannula is at least partially within the tunneling tube. The tunneling tube is configured to retract to release the device out of the tunneling tube and deploy the device in the subcutaneous pocket.

### CROSS-REFERENCE TO RELATED APPLICATION

The present application is a continuation-in-part of U.S. application Ser. No. 17/230,610, filed on Apr. 14, 2021, which claims the benefit of priority to U.S. Provisional Application Ser. No. 63/010,661, filed on Apr. 15, 2020, and is a continuation-in-part of U.S. application Ser. No. 16/844,043, filed on Apr. 9, 2020, now U.S. Pat. No. 11,432,845, which claims the benefit of priority to U.S. Provisional Application Ser. No. 62/831,286, filed on Apr. 9, 2019, each of which are incorporated herein by reference in their entireties. The present application also claims the benefit of priority to U.S. Provisional Application Ser. No. 63/508,143, filed on Jun. 14, 2023, which is incorporated herein by reference in its entirety.

### BACKGROUND

### Field of Invention

This disclosure relates to a tunneling tool for creating a subcutaneous pocket below a skin surface and implanting a device in the subcutaneous pocket.

### Discussion of the Background

Implantable devices may be implanted within a living animal (e.g., a human). Some implantable devices are implanted in subcutaneous tissue below the skin. Some implantable devices may detect the presence or amount of an analyte (e.g., glucose or oxygen) in a medium (e.g., blood or interstitial fluid) within the living animal.

Known tools for inserting an implantable device in subcutaneous tissue include a tunneling tool and an insertion tool. The tunneling tool may be used to create a tunnel and a subcutaneous pocket below a skin surface, and the insertion tool may be used to deliver the device through the tunnel and into the subcutaneous pocket.

However, when using the insertion tool, the device is typically exposed at the distal end of the insertion tool, such that the device leads the insertion tool through the tunnel below the skin surface. Often, the user has to rotate the insertion tool back and forth to force the device through the tunnel and position the device into the pocket. Such exposure and force may dislocate the device from the insertion tool before the device reaches the subcutaneous pocket and/or damage the device. Moreover, exposing the device at the distal end of the insertion tool while implanting the device may cause trauma to the inside of the pocket, which may result in more bleeding and may cause distortion in the signal transmitted from the device.

Accordingly, there is a need for an improved insertion tool to shield the device from exposure to the skin tissue during insertion and reduced trauma to the subcutaneous pocket while implanting the device. The improved insertion tool may thereby extend the longevity of the device and/or improve accuracy.

### SUMMARY

Aspects of the present invention may relate to an improved insertion tool that shields an implantable device from exposure to the skin tissue during insertion. Aspects of the improved insertion tool may reduce trauma to the subcutaneous pocket while implanting the device.

One aspect of the invention may provide an insertion tool creating a subcutaneous pocket below a skin surface and implanting a device in the subcutaneous pocket. In some aspects, the insertion tool may include a handle. In some aspects, the insertion tool may include a cannula extending from the handle, wherein the cannula comprises a passage and an opening into the passage at a distal end of the cannula. In some aspects, the cannula may be configured to move in an axial direction between an extended position and a retracted position. In some aspects, the insertion tool may include a dissector tip disposed at the distal end of the cannula, wherein the cannula and the dissector tip are configured to create the subcutaneous pocket below the skin surface. In some aspects, the insertion tool may include a rod disposed in the passage of the cannula and configured to remain stationary relative to the handle in the passage of the cannula as the cannula moves between the extended and retracted positions. In some aspects, the insertion tool may include an actuator disposed in the handle and operatively connected to the cannula such that the actuator is configured to move the cannula between the retracted and extended positions. In some aspects, the rod and the cannula may be configured such that, when the cannula is at the extended position, the rod is spatially separated from the distal end of the cannula such that the cannula holds the device in the passage of the cannula. In some aspects, the rod and cannula may be configured such that, when the cannula is at or about the retracted position, the rod is disposed proximate to the distal end of the cannula such that, as the cannula moves from the extended position to the retracted position, the rod forces the device through the opening at the distal end of the cannula, at least partially out of the cannula, and at least partially into the subcutaneous pocket.

In some aspects, the dissector tip may comprise a sleeve around at least a portion of the cannula. In some aspects, the sleeve may be configured to be pulled along the cannula from a closed position to an open position; wherein, in the closed position, the sleeve encloses the opening at the distal end of the cannula; and wherein, in the open position, the sleeve exposes the opening of the distal end of the cannula. In some aspects, the sleeve may comprise one or more perforations configured to separate when the sleeve is pulled along the cannula from the closed position to the open position.

In some aspects, the actuator may be operatively connected to the sleeve such that the actuator is configured to move the sleeve between the closed and open positions. In some aspects, the actuator may be configured to move the sleeve from the closed position to the open position before moving the cannula from the extended position to the retracted position. In some aspects, the actuator may be operatively connected to the sleeve such that the actuator is configured to move the sleeve between the closed and open positions. In some aspects, the actuator may be to move the sleeve from the closed position to the open position before moving the cannula from the extended position to the retracted position.

In some aspects, the distal end of the cannula may be bevel-shaped, and the dissector tip comprises a protrusion from the distal end of the cannula. In some aspects, the insertion tool may comprise a sleeve around at least a portion of the cannula and enclosing the opening at the distal end of the cannula. In some aspects, the sleeve may be configured to be pulled along the cannula from a closed position to an open position; wherein, in the closed position, the sleeve encloses the opening at the distal end of the cannula; and wherein, in the open position, the sleeve exposes the opening at the distal end of the cannula. In some aspects, the sleeve may comprise one or more perforations configured to separate when the sleeve is pulled along the cannula from the closed position to the open position.

In some aspects, the dissector tip may be blunt-shaped. In some aspects, the distal end of the cannula may be bevel-shaped, and the dissector tip may comprise a flap coupled to the cannula, wherein the flap is configured to cover the opening at the distal end of the cannula. In some aspects, the flap may be configured to pivot between a closed position, in which the flap encloses the opening of the distal end of the cannula, and an open position, in which the flap is spatially separated from the distal end of the cannula and exposes the opening at the distal end of the cannula. In some aspects, the actuator may comprise a track extending along a portion of the handle and a slider knob configured to slide along the track and cause the rod to move between the retracted and extended positions.

Another aspect of the invention may provide an insertion tool for creating a subcutaneous pocket below a skin surface and implanting a device in the subcutaneous pocket. In some aspects, the insertion tool may include a handle defining a cavity therein. In some aspects, the insertion tool may comprise a tunneling tube extending from a first end of the handle and defining a passage opening into the cavity of the handle, and the tunneling tube is configured to move in an axial direction between an extended position and a retracted position. In some aspects, the dissector may comprise a blunt tip configured to move between a retracted position, wherein the blunt tip is disposed in the cavity of the handle, and an extended position, wherein the blunt tip protrudes out of a distal end of the tunneling tube, and the dissector is configured to create the subcutaneous pocket. In some aspects, the insertion tool may comprise an insertor comprising a cannula configured to move between a retracted position, wherein the cannula is disposed in the cavity of the handle, and an extended position, wherein the cannula is at least partially disposed in the passage of the tunneling tube. In some aspects, the insertion tool may comprise an actuator disposed in the handle and operatively linked to the cannula, the dissector, and the insertor such that the actuator is configured to trigger the cannula, the blunt tip, and the cannula to move between the retracted and extended positions. In some aspects, the tunneling tube and the cannula may be configured such that, when the tunneling tube and the cannula are at the extended position, the cannula holds the device in the passage of the cannula. In some aspects, the tunneling tube and the cannula may be configured such that, when the tunneling tube is at or about the retracted position, the cannula moves toward the retracted position and releases the device out of the tunneling tube to deploy the device in the subcutaneous pocket.

In some aspects, the dissector may be configured to pivot from an operating position disposed along a first axis defined by the tunneling tube to an idle position disposed along a second axis, and the inserter is configured to pivot from an idle position disposed along a third axis to an operating position disposed along the first axis, wherein the second axis extends at a first acute angle with respect to the first axis, and third axis extends at a second acute angle with respect to the first axis. In some aspects, the handle may comprise a hinge pivotably coupled to the dissector and the inserter. In some aspects, the inserter may be configured to pivot between the operating and idle positions when the cannula tube is set in the retracted position, and the cannula tube is configured to move between the retracted and extended positions when the inserter is set at the operating position.

In some aspects, the actuator may comprise a track extending along a portion of the handle and a slider knob configured to slide along the track and cause the tunneling tube, the blunt tip, and the cannula to move between the retracted and extended positions. In some aspects, the dissector may comprise a first rod configured to move along the cavity of the handle and the passage of the tunneling tube, and the blunt tip is coupled to the first rod such that the first rod is configured to move the blunt tip along the cavity of the handle and the passage of the tunneling tube between the retracted and extended positions. In some aspects, the insertor may comprise a second rod configured to move along the cavity of the handle and the passage of the tunneling tube, and the cannula comprises a first end configured to hold and release the device and a second end coupled to the second rod such that the second rod is configured to move the cannula along the cavity of the handle and the passage of the tunneling tube between the retracted and extended positions.

Yet another aspect of the invention may provide a method of using an insertion tool to create a subcutaneous pocket below a skin surface and implant a device in the subcutaneous pocket. In some aspects, the method may comprise a step of inserting a cannula of the insertion tool and a dissector tip disposed at a distal end of the cannula into an incision in the skin surface such that the dissector tip and the cannula create the subcutaneous pocket. In some aspects, the method may comprise a step of moving the cannula in an axial direction from an extended position to a retracted position such that a rod disposed along a passage of the cannula forces the device through an opening at the distal end of the cannula, at least partially out of the passage of the cannula, and at least partially into the subcutaneous pocket.

In some aspects, the method may further comprise loading the device into the passage of the cannula. In some aspects, the method may further comprise after loading the device into the passage and before inserting the cannula and the dissector tip, pulling a sleeve around the distal end of the cannula to enclose the opening in the distal end of the cannula. In some aspects, the method may further comprise before the step of loading, pulling a sleeve received around the cannula toward the handle to expose the opening in the distal end of the cannula. In some aspects, the method may further comprise pulling the cannula away from the subcutaneous pocket.

In some aspects, the method may further comprise using an actuator disposed in the handle to move the cannula from the extended position to the retracted position. In some aspects, the method may further comprise pulling a sleeve along the cannula from a closed position, in which the sleeve encloses the opening at the distal end of the cannula, to an open position, in which the sleeve exposes the opening at the distal end of the cannula. In some aspects, the method may further comprise a flap covers the opening at the distal end of the cannula, and moving the rod along the passage of the cannula from the retracted position to the extended position spatially separates the flap from the distal end of the cannula and exposes the opening at the distal end of the cannula.

Still another aspect of the invention may provide an insertion tool for creating a subcutaneous pocket below a skin surface and implanting a device in the subcutaneous pocket. The insertion tool may include a handle defining a cavity therein. The insertion tool may include a tunneling tube defining a passage opening into the cavity of the handle. The insertion tool may include a dissector comprising a blunt tip. The dissector may be configured to: (i) create the subcutaneous pocket; (ii) move from a retracted position, wherein the blunt tip is disposed in the cavity of the handle, and an extended position, wherein the blunt tip protrudes out of a distal end of the tunneling tube; and (iii) rotate from an operating position disposed on a longitudinal axis of the tunneling tube to an idle position disposed off the longitudinal axis of the tunneling tube. The insertion tool may include an insertor comprising a cannula. The inserter may be configured to: (i) hold the device in a passage of the cannula; (ii) rotate from an idle position disposed off the longitudinal axis of the tunneling tube to an operating position disposed on the longitudinal axis of the tunneling tube; and (iii) move from a retracted position to an extended position and release the device out of the tunneling tube to deploy the device in the subcutaneous pocket.

In some aspects, the inserter may be configured to hold hydration fluid in the passage of the cannula. In some aspects, the handle may include a hinge rotatably coupled to the dissector and the inserter. In some aspects, the inserter may be configured to move from the retracted position to the extended position when the inserter is set at the operating position. In some aspects, the actuator may include a slider knob configured to cause the inserter and the dissector to move between the retracted and extended positions. In some aspects, the insertion tool may further include a mechanism that, after the inserter is rotated to the operating position, prevents backward movement of the slider knob of the actuator so that the cannula of the inserter is capable of only moving toward the extended position of the inserter. In some aspects, the mechanism may be a ratchet.

In some aspects, the dissector may include a first rod configured to move along the cavity of the handle and the passage of the tunneling tube, and the blunt tip may be coupled to the first rod such that the first rod is configured to move the blunt tip along the cavity of the handle and the passage of the tunneling tube between the retracted and extended positions. In some aspects, the insertor may include a second rod configured to move along the cavity of the handle and the passage of the tunneling tube, and the cannula may include a first end configured to hold and release the device and a second end coupled to the second rod such that the second rod is configured to move the cannula along the cavity of the handle and the passage of the tunneling tube between the retracted and extended positions.

Yet another aspect of the invention may provide a method for creating a subcutaneous pocket below a skin surface and implanting a device in the subcutaneous pocket. The method may include using a dissector of an insertion tool with the dissector in an extended position in which a blunt tip of the dissector protrudes out of a distal end of a tunneling tube to create the subcutaneous pocket, and the tunneling tube may define a passage opening into a cavity of the handle. The method may include moving the dissector from the extended position to a retracted position in which the blunt tip is disposed in the cavity of the handle. The method may include rotating the dissector from an operating position disposed on a longitudinal axis of the tunneling tube to an idle position disposed off the longitudinal axis of the tunneling tube. The method may include using an inserter of the insertion tool to hold the device in a passage of a cannula of the inserter. The method may include rotating the inserter from an idle position disposed off the longitudinal axis of the tunneling tube to an operating position disposed on the longitudinal axis of the tunneling tube. The method may include moving the inserter from a retracted position to an extended position, and moving the inserter from the retracted positon to the extended position may release the device out of the tunneling tube and deploys the device in the subcutaneous pocket.

In some aspects, the method may further include using the inserter to hold hydration fluid in the passage of the cannula. In some aspects, rotating the dissector and rotating the inserter may include using a hinge rotatably coupled to the dissector and the inserter. In some aspects, moving the inserter from the retracted position to the extended position may include moving the inserter from the retracted position to the extended position while the inserter is in the operating position. In some aspects, moving the dissector from the extended position to the retracted position and moving the inserter from the retracted position to the extended position may include using a slider knob to cause the inserter and the dissector to move. In some aspects, moving the inserter from the retracted position to the extended position may include preventing backward movement of the slider knob of the actuator so that the cannula of the inserter is capable of only moving toward the extended position.

In some aspects, the dissector may include a first rod configured to move along the cavity of the handle and the passage of the tunneling tube, and the blunt tip may be coupled to the first rod such that the first rod is configured to move the blunt tip along the cavity of the handle and the passage of the tunneling tube between the retracted and extended positions. In some aspects, the insertor may include a second rod configured to move along the cavity of the handle and the passage of the tunneling tube, and the cannula may include a first end configured to hold and release the device and a second end coupled to the second rod such that the second rod is configured to move the cannula along the cavity of the handle and the passage of the tunneling tube between the retracted and extended positions.

Still another aspect of the invention may provide a method for creating a subcutaneous pocket below a skin surface and implanting a device in the subcutaneous pocket. The method may include using a dissector of an insertion tool with the dissector in an extended position in which a blunt tip of the dissector protrudes out of a distal end of a tunneling tube to create the subcutaneous pocket. The tunneling tube may define a passage opening into a cavity of the handle. The method may include moving the dissector from the extended position to a retracted position in which the blunt tip is disposed in the cavity of the handle. The method may include rotating the dissector from an operating position disposed on a longitudinal axis of the tunneling tube to an idle position disposed off the longitudinal axis of the tunneling tube. The method may include rotating an inserter of the insertion tool from an idle position disposed off the longitudinal axis of the tunneling tube to an operating position disposed on the longitudinal axis of the tunneling tube. The inserter may hold the device in a passage of a cannula of the inserter. The method may include moving the inserter from a retracted position to an extended position in which the cannula is at least partially in the tunneling tube. The method may include retracting the tunneling tube to release the device out of the tunneling tube and deploy the device in the subcutaneous pocket.

In some aspects, the method may further include moving a slider knob of an actuator of the insertion tool in a first direction to move the dissector from the extended position to the retracted position in which the blunt tip is disposed in the cavity of the handle, moving the slider knob in a second direction to move the inserter from the retracted position to the extended position, and moving the slider knob in the first direction to retract the tunneling tube. In some aspects, the method may further include using a stopper of the insertion tool to prevent movement of the slider knob while the dissector is used create the subcutaneous pocket and removing the stopper of the insertion tool, and moving the slider knob in the first direction to move the dissector from the extended position to the retracted position in which the blunt tip is disposed in the cavity of the handle may occur after removal of the stopper.

In some aspects, the method may further include using tubing connected to the cannula to provide hydration fluid to the passage of the cannula. In some aspects, the tubing may connect a hydration fluid container and the cannula, and the method may further include using the hydration fluid container to provide the hydration fluid to the passage of the cannula via the tubing.

In some aspects, rotating the dissector and rotating the inserter may include using a swing rotatably coupled to the dissector and the inserter. In some aspects, moving the inserter from the retracted position to the extended position may include moving the inserter from the retracted position to the extended position while the inserter is in the operating position. In some aspects, the dissector may include a first rod configured to move along the cavity of the handle and the passage of the tunneling tube, and the blunt tip may be coupled to the first rod such that the first rod is configured to move the blunt tip along the cavity of the handle and the passage of the tunneling tube between the retracted and extended positions.

In some aspects, the insertor may include a second rod configured to move along the cavity of the handle and the passage of the tunneling tube, and the cannula may include a first end configured to hold and release the device and a second end coupled to the second rod such that the second rod is configured to move the cannula along the cavity of the handle and the passage of the tunneling tube between the retracted and extended positions. In some aspects, the cannula may retract with tunneling tube, and the method may further include using the second rod to force the device at least partially out of the cannula and the tunneling tube.

Yet another aspect of the invention may provide an insertion tool for creating a subcutaneous pocket below a skin surface and implanting a device in the subcutaneous pocket. The insertion tool may include a handle, a tunneling tube, a dissector, and an insertor. The handle may define a cavity therein. The tunneling tube may define a passage opening into the cavity of the handle. The dissector may include a blunt tip and may be configured to: (i) create the subcutaneous pocket; (ii) move from an extended position, wherein the blunt tip protrudes out of a distal end of the tunneling tube, to a retracted position, wherein the blunt tip is disposed in the cavity of the handle; and (iii) rotate from an operating position disposed on a longitudinal axis of the tunneling tube to an idle position disposed off the longitudinal axis of the tunneling tube. The insertor may include a cannula and configured to: (i) hold the device in a passage of the cannula; (ii) rotate from an idle position disposed off the longitudinal axis of the tunneling tube to an operating position disposed on the longitudinal axis of the tunneling tube; and (iii) move from a retracted position to an extended position in which the cannula is at least partially within the tunneling tube. The tunneling tube is configured to retract to release the device out of the tunneling tube and deploy the device in the subcutaneous pocket.

In some aspects, the actuator may include a slider knob configured to cause the inserter and the dissector to move between the retracted and extended positions. In some aspects, the insertion tool may further include a removable stopper configured to prevent movement of the slider knob. In some aspects, the insertion tool may further include tubing connected to the cannula and configured to provide hydration fluid to the passage of the cannula.

In some aspects, the handle may include a swing rotatably coupled to the dissector and the inserter. In some aspects, the inserter may be configured to move from the retracted position to the extended position when the inserter is set at the operating position. In some aspects, the dissector may include a first rod configured to move along the cavity of the handle and the passage of the tunneling tube, and the blunt tip may be coupled to the first rod such that the first rod is configured to move the blunt tip along the cavity of the handle and the passage of the tunneling tube between the retracted and extended positions.

In some aspects, the insertor may include a second rod configured to move along the cavity of the handle and the passage of the tunneling tube, and the cannula may include a first end configured to hold and release the device and a second end coupled to the second rod such that the second rod is configured to move the cannula along the cavity of the handle and the passage of the tunneling tube between the retracted and extended positions. In some aspects, the cannula may be configured to retract with tunneling tube, and the second rod is configured to force the device at least partially out of the cannula and the tunneling tube.

Still another aspect of the invention may provide a package including an insertion tool, a hydration fluid container, and tubing connecting the hydration fluid container to the insertion tool and configured to provide hydration fluid from the hydration fluid container to the insertion tool.

In some aspects, the insertion tool may include a cannula, a device in a passage of the cannula, and a loading port. In some aspects, the hydration fluid container may be configured to provide the hydration fluid into the cannula of the insertion tool via the tubing and the loading port. In some aspects, the device in the passage of the cannula may include a hydrogel, and the hydration fluid may hydrate the hydrogel of the device. In some aspects, the tubing may be removably connected to the cannula.

In some aspects, the insertion tool may include a tunneling tube and a dissector tip protruding from the tunneling tube. In some aspects, the package may further include a base and a removable cover. In some aspects, the base and the cover may enclose the insertion tool, the hydration fluid container, and the tubing.

In some aspects, the hydration fluid container may include a syringe. In some aspects, the tubing may be luer lock tubing.

Still another aspect of the invention may provide a method including using a hydration fluid container to provide a hydration fluid to a device in a passage of a cannula of an insertion tool via tubing connecting the hydration fluid container and the insertion tool.

In some aspects, the method may further include opening a package enclosing the insertion tool, the hydration fluid container, and the tubing connecting the hydration fluid container and the insertion tool. In some aspects, opening the package may include removing a cover of the package from a base of the package.

In some aspects, using the hydration fluid container to provide the hydration fluid may include activating a plunger of the hydration fluid container. In some aspects, the insertion tool may include a tunneling tube and a dissector tip protruding from the tunneling tube. In some aspects, the method may further include using the insertion tool to create a subcutaneous pocket below a skin surface and implant the device in the subcutaneous pocket. In some aspects, the hydration fluid container may provide the hydration fluid to the device in the passage of the cannula of the insertion tool via the tubing and a loading port of the insertion tool.

Yet another aspect of the invention may provide a package including a base and a stand. The base may include a cavity. The base may be configured to support a product at least partially within the cavity. The stand may be configured to (i) fit at least partially within the cavity of the base, (ii) when the stand is at least partially within the cavity of the base, hold the product at least partially within the cavity of the base between the stand and a bottom surface of the cavity of the base, (iii) attach to an exterior bottom surface of the base, and (iv) when the stand is attached to the exterior bottom surface of the base, define an angle of tilt of the product supported within the cavity of the base.

In some aspects, the package may further include the product. In some aspects, the product may be an insertion tool. In some aspects, the insertion tool may include a cannula and a device in a passage of the cannula, and the angle of tilt defined by the stand may be such that hydration fluid in the cannula stays in the cannula around the device. In some aspects, the base may include a passage through which tubing is capable of passing, and the tubing may be configured to connect to a hydration fluid container to the product and provide hydration fluid from the hydration fluid container to the product.

In some aspects, the base may be configured to support at least a portion of the bottom of the product and at least a portion of sides of the product. In some aspects, the stand may be configured to, when the stand is at least partially within the cavity of the base, hold the product at least partially within the cavity of the base by contacting at least a portion of a top of the product supported at least partially within the cavity.

In some aspects, the package may further include a removable cover, and the base and the cover may enclose the product and the stand.

Still another aspect of the invention may provide a method including using a base to support a product at least partially within a cavity of the base. The method may include using a stand at least partially within the cavity of the base to hold the product at least partially within the cavity of the base between the stand and a bottom surface of the cavity of the base. The method may include removing the stand from the cavity of the base. The method may include attaching the stand to an exterior bottom surface of the base, and the stand attached to the exterior bottom surface of the base may define an angle of tilt of the product supported within the cavity of the base.

In some aspects, the method may further include using tubing to connect a hydration fluid container to the product and provide hydration fluid from the hydration fluid container to the product. In some aspects, using the tubing to connect the hydration fluid container to the product comprises passing the tubing through a passage of the base.

In some aspects, the method may further include removing a cover from the base, and the base and the cover may enclose the product and the stand before the cover is removed.

Yet another aspect of the invention may include a method of loading a device into an insertion tool. The method may include moving a slider of a loading tool from a forward position to a rearward position so that the slider pulls the device into a passage of the insertion tool. The method may include moving the slider from the rearward position to the forward position so that the slider pushes the device forward, out of the passage of the loading tool, and into a cannula of the insertion tool. The method may include removing the loading tool from the insertion tool and leaving the device in the cannula of the insertion tool.

Further variations encompassed within the insertion tools and methods are described in the detailed description of the invention below.

### BRIEF DESCRIPTION OF THE DRAWINGS

The accompanying drawings, which are incorporated herein and form part of the specification, illustrate various aspects of the subject matter of this disclosure. In the drawings, like reference numbers indicate identical or functionally similar elements.

FIG. 1 is a schematic view of an insertion tool embodying aspects of the present disclosure.

FIG. 2A is a perspective view of a dissector pull sleeve actuated tip in a closed position with the push rod retracted embodying aspects of the present disclosure.

FIG. 2B is a cross-sectional side view of the pull sleeve actuated tip in the closed position with the push rod retracted embodying aspects of the present disclosure.

FIG. 2C is a perspective view of the dissector pull sleeve actuated tip in an open position with the push rod retracted embodying aspects of the present disclosure.

FIG. 2D is a cross-sectional side view of the dissector pull sleeve actuated tip in the open position with the push rod retracted embodying aspects of the present disclosure.

FIG. 2E is a perspective view of the dissector pull sleeve actuated tip in an open position with the push extended embodying aspects of the present disclosure.

FIG. 2F is a cross-sectional side view of the dissector pull sleeve actuated tip in the open position with the push rod extended embodying aspects of the present disclosure.

FIG. 3A is a cross-sectional side view of an insertion tool dissector pull sleeve actuated bevel-shaped tip in a closed position with the push rod retracted embodying aspects of the present disclosure.

FIG. 3B is a perspective view of the insertion tool dissector pull sleeve actuated bevel-shaped tip in the closed position with the push rod retracted embodying aspects of the present disclosure.

FIG. 3C is a perspective view of the insertion tool dissector pull sleeve actuated bevel-shaped tip in an open position with the push rod retracted embodying aspects of the present disclosure.

FIG. 4 is a cross-sectional side view of an insertion tool dissector device-supported tip in a closed position with the push rod retracted embodying aspects of the present disclosure.

FIG. 5 is a cross-sectional side view of an insertion tool dissector device-supported bevel-shaped tip in a closed position with the push rod retracted embodying aspects of the present disclosure.

FIG. 6A is a cross-sectional side view of an insertion tool dissector plug-covered bevel-shaped tip in the closed and retracted position embodying aspects of the present disclosure.

FIG. 6B is a perspective view of the insertion tool dissector plug-covered bevel-shaped tip in an open position with the push rod retracted embodying aspects of the present disclosure.

FIG. 7A is a cross-sectional side view of an insertion tool dissector having a device as a nose cone and with the push rod in a retracted position embodying aspects of the present disclosure.

FIG. 7B is a cross-sectional side view of the insertion tool dissector having the device as the nose cone with the push rod in an extended position embodying aspects of the present disclosure.

FIG. 7C is a perspective view of the insertion tool dissector having the device as the nose cone with the push rod in the retracted position embodying aspects of the present disclosure.

FIG. 8A is a cross-sectional side view of an insertion tool with the dissector tip set in the extended position and the cannula set in the retracted position embodying aspects of the present disclosure.

FIG. 8B is a cross-sectional side view of an insertion tool with the dissector tip set in the retracted position and the cannula set in the extended position embodying aspects of the present disclosure.

FIG. 9A is a schematic view of an insertion tool with the dissector tip set in the extended position and the cannula set in the retracted position embodying aspects of the present disclosure.

FIG. 9B is a schematic view of an insertion tool with the dissector tip set in the retracted position and the cannula set in the extended position embodying aspects of the present disclosure.

FIG. 10 is a schematic view of an insertion tool embodying aspects of the present disclosure.

FIG. 11 is a flow chart of a method of creating a subcutaneous pocket and implanting a device in the subcutaneous pocket embodying aspects of the present disclosure.

FIG. 12A is perspective view of an insertion tool embodying aspects of the present disclosure.

FIGS. 12B and 12C are perspective views of components within an insertion tool with the dissector tip set in the extended position and the cannula set in the retracted position embodying aspects of the present disclosure.

FIG. 12D is a schematic view of components within an insertion tool with the dissector tip set in the retracted position and the cannula set in the retracted position embodying aspects of the present disclosure.

FIG. 12E is a schematic view of components within an insertion tool with the dissector tip set in the retracted position and the cannula set in the extended position embodying aspects of the present disclosure.

FIG. 13 is a flow chart of a method of creating a subcutaneous pocket and implanting a device in the subcutaneous pocket embodying aspects of the present disclosure.

FIGS. 14A and 14B are perspective views of an insertion tool embodying aspects of the present disclosure.

FIGS. 14C and 14D are perspective views of components of an insertion tool embodying aspects of the present disclosure.

FIG. 15A is a perspective view of a package including an insertion tool and hydration fluid container embodying aspects of the present disclosure.

FIG. 15B is a perspective view of an insertion tool with a dissector tip in an extended position and a stopper preventing movement of a slider knob embodying aspects of the present disclosure.

FIG. 15C is a perspective view of an insertion tool with the stopper removed embodying aspects of the present disclosure.

FIG. 15D is a perspective view of an insertion tool with the dissector tip in a retracted position embodying aspects of the present disclosure.

FIG. 15E is a perspective view of an insertion tool with a cannula in an extended position embodying aspects of the present disclosure.

FIG. 15F is a perspective view of an insertion tool with a cannula and tunneling tube in a retracted position embodying aspects of the present disclosure.

FIGS. 16A and 16B are side views of an insertion tool embodying aspects of the present disclosure.

FIG. 16C is a perspective view of a stopper of an insertion tool embodying aspects of the present disclosure.

FIGS. 16D-16F illustrate perspective, top, and perspective views, respectively, an insertion tool including an extension embodying aspects of the present disclosure.

FIG. 16G is a side view of an insertion tool embodying aspects of the present disclosure.

FIGS. 16H and 16I are perspective views of a cannula support embodying aspects of the present disclosure.

FIGS. 17A and 17B illustrate a package embodying aspects of the present disclosure.

FIGS. 17C-17F illustrate a base of a package embodying aspects of the present disclosure.

FIGS. 17G-17K illustrate a stand of a package embodying aspects of the present disclosure.

FIGS. 18A-18C illustrate perspective, cross-sectional perspective, and cross-sectional side views, respectively, of an insertion tool, tubing, and hydration fluid container embodying aspects of the present disclosure.

FIGS. 18D-18F illustrate perspective, cross-sectional perspective, and cross-sectional side views of an insertion tool with a dissector tip in an extended position and a stopper preventing movement of a slider knob embodying aspects of the present disclosure.

FIGS. 18G-18I illustrate perspective, cross-sectional perspective, and cross-sectional side views of an insertion tool with the stopper removed and the dissector tip in a retracted position embodying aspects of the present disclosure.

FIGS. 18J-18L illustrate perspective, cross-sectional perspective, and cross-sectional side views of an insertion tool with an inserter in an extended position embodying aspects of the present disclosure.

FIGS. 18M-180 illustrate perspective, cross-sectional perspective, and cross-sectional side views of an insertion tool with a tunneling tube in a retracted position embodying aspects of the present disclosure.

FIG. 19 is a flowchart illustrating a method of creating a subcutaneous pocket and implanting a device in the subcutaneous pocket embodying aspects of the present disclosure.

FIG. 20 is a flowchart illustrating a method of creating a subcutaneous pocket and implanting a device in the subcutaneous pocket embodying aspects of the present disclosure.

FIG. 21 is a flowchart illustrating a method embodying aspects of the present disclosure.

FIG. 22A illustrates a loading tool embodying aspects of the present disclosure, and FIGS. 22B-22E illustrate steps of a loading process for using the loading tool to load a device into a cannula of an insertion tool embodying aspects of the present disclosure.

### DETAILED DESCRIPTION

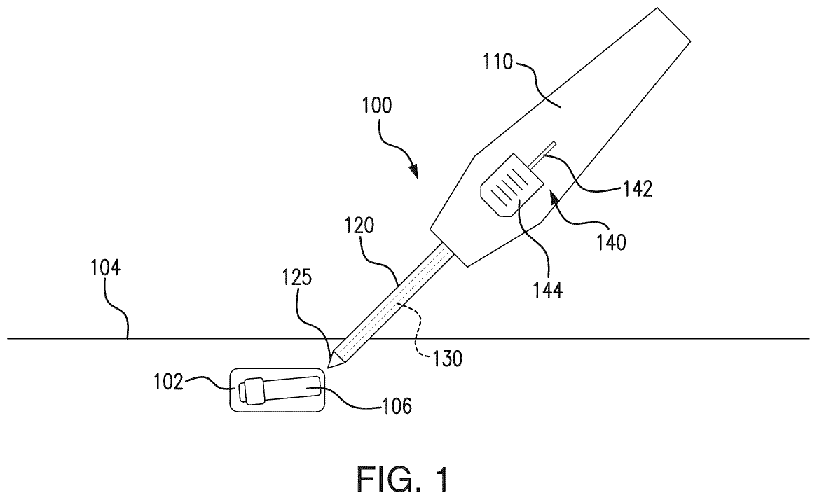

FIG. 1 is a schematic view illustrating an exemplary insertion tool 100 embodying aspects of the present disclosure. In some aspects, the insertion tool 100 may be for creating a subcutaneous pocket 102 below a skin surface 104 and implanting a device 106 in the subcutaneous pocket 102. In some aspects, the device 106 may include an RFID chip. In some aspects, the device 106 may include a sensor, such as, for example and without limitation, an analyte sensor (e.g., glucose sensor) and/or a temperature sensor. In some aspects, the insertion tool 100 may include one or more of a handle 110, a cannula 120, a dissector tip 125, a rod 130, and an actuator 140.

In some aspects, the cannula 120 may extend from the handle 110. In some aspects, the cannula 120 may define a passage (e.g., through which the device 106 may pass). In some aspects, the cannula 120 and dissector tip 125 may be configured to create the subcutaneous pocket below 102 the skin surface 104 by inserting the dissector tip 125 and cannula 120 into an incision (not shown) made in the skin surface 104. In some aspects, the cannula 120 may be configured to move in an axial direction between an extended position and a retracted position. In some aspects, when the cannula 120 is set at the extended position, the cannula 120 and the dissector tip 125 may be configured to create the subcutaneous pocket 102 below the skin surface 104. In some aspects, movement of the cannula 120 to the retracted position may deploy the device 106 from within the passage of the cannula 120 into the subcutaneous pocket 102. In some aspects, when the cannula 120 is set at the extended position, a substantial portion of the cannula 120 may be disposed outside the handle 110. In some aspects, when the cannula 120 is set at the retracted position, a portion or all of the cannula 120 may be disposed in a cavity (not shown) of the handle 110.

In some aspects, the rod 130 may be disposed (at least partially) in the passage of the cannula 120. In some aspects, movement of the cannula 120 between the extended and retracted positions may be relative to the rod 130, which the rod 130 may be configured to remain stationary relative to the handle 110 in the passage of the cannula 120 as the cannula 120 moves between the extended and retracted positions. In some aspects, when the cannula 120 is set in the extended position, the rod 130 may be spatially separated from a distal end of the cannula 120 so that the passage of the cannula 120 holds the device 106. In some aspects, when the cannula 120 is moved from the extended position to the retracted position, the distal end of the cannula 120 may move toward the distal end of the rod 130, which may cause the rod 130 to act as a backstop and force the device 106 within the passage of the cannula 120 through an opening at the distal end of the cannula 120, at least partially out of the cannula 120, and at least partially into the subcutaneous pocket 120. In some aspects, while or after retracting the cannula 120, a user may pull the handle 110 away from the pocket, leaving the device 106 in the subcutaneous pocket 120.

In some aspects, the actuator 140 may be disposed in the handle 110 and may be operatively connected to the cannula 120. In some aspects, the actuator 140 may be configured to move the cannula 120 between the retracted and extended positions. In some aspects, the actuator 140 may include a track 142 extending along the handle 110 and a slider knob 144 configured to slide along the track 142. In some aspects, movement of the slider knob 144 of the actuator 140 may force the cannula 120 to move between the extended and retracted positions. In some aspects, the cannula 120 may be configured to move from the extended position to the retracted position by sliding the slider knob 144 along the track 142 in a direction away from the cannula 120. In some aspects, the cannula 120 may be configured to move from the retracted position to the extended position by sliding the slider knob 144 along the track 142 in a direction toward the cannula 120. In other aspects (not shown), the actuator 140 may additionally or alternatively include other mechanisms, such as, for example, a spring, a solenoid, or a motor, to bring about movement of the cannula 120.

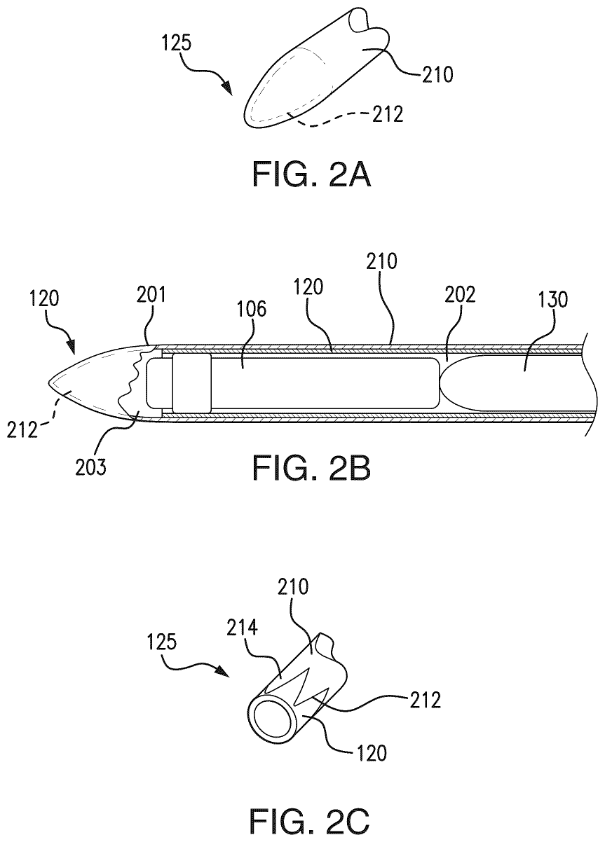

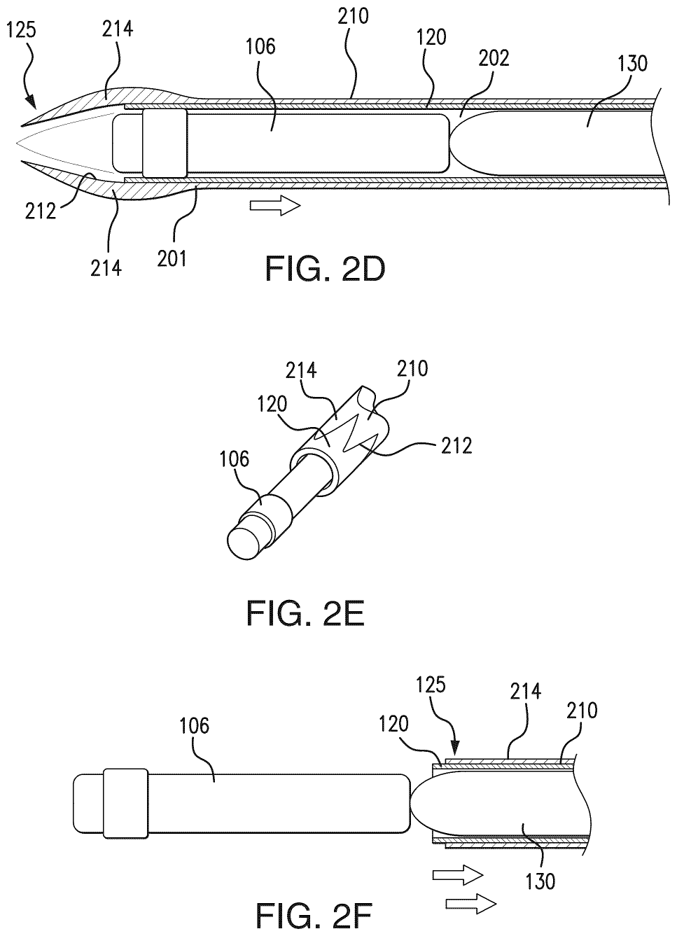

FIGS. 2A-7C illustrate various exemplary aspects of the cannula 120 and the inserter 130 that may be implemented with the insertion tool 100 according to the present disclosure.

In some aspects, as shown in FIGS. 2B and 2D, the cannula 120 may have a tubular shape and may define a passage 202 extending along a longitudinal axis of the cannula 120. In some aspects, the cannula 120 may comprise a distal end 201 defining an opening 203 into the passage 202. In some aspects, the distal end 201 of the cannula 120 may move in an axial direction away from the handle 110 when the cannula 120 is moving toward the extended position. In some aspects, the distal end 201 of the cannula 120 may move in an axial direction toward the handle 110 when the cannula 120 is moving toward the retracted position. In some non-limiting aspects, the cannula 120 may be comprised of, for example and without limitation, stainless steel. In some other non-limiting aspects, the cannula 120 may be comprised of a polymeric material, such as, for example and without limitation, PC (polycarbonate), PPSU (polymer polyphenyl sulfone), PEEK (polyether ether ketone), PES (Polyethersulfone), POLY (polyarylamide) . . . .

In some aspects, as shown in FIGS. 2A-2F, the dissector tip 125 may comprise a sleeve 210 around at least a portion of the cannula 120. In some aspects, the sleeve 210 may comprise one or more perforations 212 (e.g., one or more weakened portions or score lines) defining two or more portions 214 of the sleeve 210 proximate to the distal end 201 of the cannula 120. In some aspects, the one or more perforations 212 may be separated by pulling the sleeve 210 away from the distal end 201 of the cannula 120 and toward the handle 110. In some aspects, as shown in FIGS. 2C-2E, the separation at the one or more perforations or weakened portions 212 may result in the portions 214 of the sleeve 210 separating from one another. In some aspects, the separated portions 214 of the sleeve 210 may expose the opening 203 into the passage 202 of the cannula 120. In some non-limiting aspects, the sleeve 210 may be comprised of a polymeric material, such as, for example and without limitation, PU (polyurethane), PVC (polyvinyl chloride), PTFE (Polytetrafluoroethylene).

In some aspects, the sleeve 210 may be operatively connected to the actuator 140. In some aspects, the sleeve 210 may be configured to be pulled along the cannula 120 from a closed position to an open position by sliding the slider knob 144 along the track 142 in a direction away from the cannula 120. In some aspects, as shown in FIGS. 2A and 2B, when the sleeve 210 is in the closed position, the sleeve 210 may enclose the opening 203 of the distal end 201 of the cannula 120. In some aspects, when the sleeve 210 is moved to the open position, the sleeve 210 may be pulled away from the distal end 201 of the cannula 120 and toward the handle 110. In some aspects, as shown in FIGS. 2C-2F, moving the sleeve 210 toward the open position may separate the one or more perforations or weakened portions 212 and expose the opening 203 of the distal end 201 of the cannula 120. In some aspects, when the sleeve 210 is pulled toward the handle 110 of the insertion tool 100, the distal end 201 of the cannula 120 may apply a sufficient amount of force against the sleeve to separate the portions 214 of the sleeve 210. In some aspects, the sleeve 210 may be configured to be moved toward the open position before the cannula 120 moves toward the retracted position to ensure that the distal end 201 of the cannula 120 engages the portions 214 of the sleeve 210 with a sufficient amount of force to separate or rupture the perforations or weakened portions 212.

In some aspects, as shown in FIGS. 2B-2F, the distal end 201 of the cannula 120 may be blunt-shaped. However, in other aspects, the distal end 201 of the cannula 120 may form other shapes (e.g., a bevel shape) configured to create a tunnel and pocket below the skin surface.

In some aspects, as shown in FIGS. 2B, 2D, and 2F, the rod 130 may be disposed, at least partially, in the passage 202 of the cannula 120. In some aspects, the rod 130 may be configured to remain stationary (relative to the handle 110) in the passage 202 of the cannula 120 as the cannula 120 moves between the retracted position and the extended position. In some aspects, as shown in FIG. 2B, when the cannula 120 is set at the extended position, the rod 130 may be spatially separated from the distal end 201 of the cannula 120 such that the passage 202 of the cannula 120 receives and/or holds the device 106 proximate to the distal end 201. In some aspects, as shown in FIG. 2F, when the cannula 120 is set at the retracted position, the rod 130 may be disposed proximate the distal end 201 of the cannula 120 or may protrude through the distal end 201 of the cannula 120. In some aspects, when the cannula 120 is moved from the extended position to the retracted position, the rod 130 may act as a backstop and force the device 106 through the opening 203 at the distal end 201 of the cannula 120 and at least partially out of the cannula 120.

In some aspects, the rod 130 may be solid or hollow. In some aspects, the rod 130 may be comprised of a rigid material, such as, for example and without limitation, stainless steel, polymeric material, such as ABS, Polycarbonate, Polyarylamide.

In some aspects, the insertion tool 100 may create the subcutaneous pocket 102 below the skin surface 104 by inserting the cannula 120 at the extended position with the dissector tip 125 through an incision in the skin surface. In some aspects, when the dissector tip 125 and the cannula 120 are inserted into the incision and while the dissector tip 125 and the cannula 120 create the subcutaneous pocket 102, the sleeve 210 may be set at the closed position (as shown in FIGS. 2A and 2B), and the sleeve 210 may enclose the device 106 in the passage 202 of the cannula 120. In some aspects, after creating the subcutaneous pocket 102, the sleeve 210 may be moved (e.g., by pulling the sleeve 210 toward the handle 110) to the open position in which the opening 203 at the distal end 201 of the cannula 120 is exposed (as shown in FIGS. 2C and 2D). In some aspects, after the moving the sleeve 210 to the open position, the insertion tool 100 may implant the device 106 by moving the cannula 120 from the extended position toward the retracted position. In some aspects, as the cannula 120 moves toward the retracted position, the rod 130 may abut against the device 106, act as a backstop, and propel the device 106 through the opening 203 at the distal end 201 of the cannula 120, at least partially out of the cannula 120, and at least partially into the subcutaneous pocket 102 (as shown in FIGS. 2E and 2F). In some aspects, after moving the cannula 120 to the retracted position (or simultaneously therewith), the handle 110 may be used to pull the cannula 120 out of the subcutaneous pocket 102, and the device 106 may be left in the subcutaneous pocket 102.

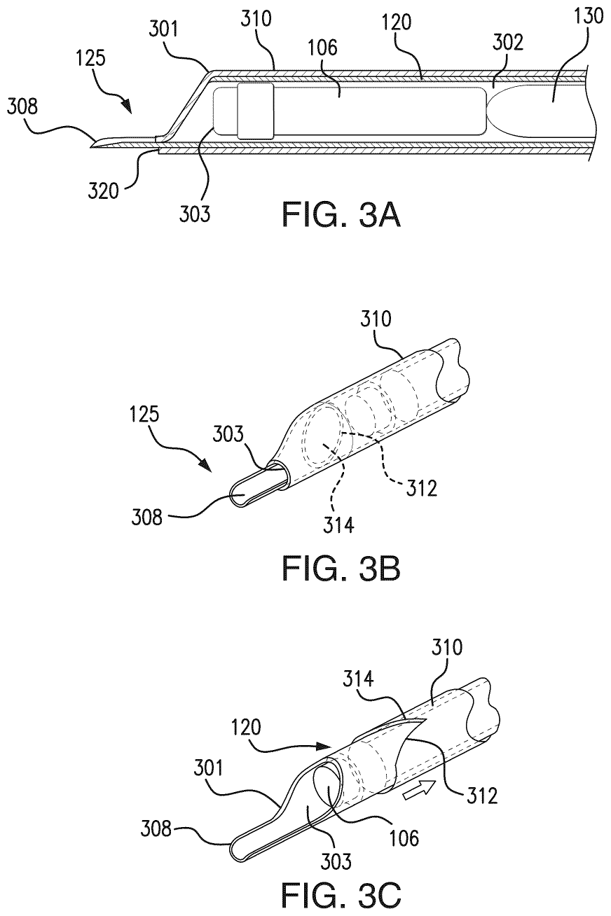

In some aspects, as shown in FIGS. 3A-3C, the cannula 120 may have a bevel-shaped distal end 301. In some aspects, the bevel-shaped distal end 301 may define an opening 303 into the passage 302 of the cannula 120. In some aspects, the dissector tip 125 may include a protrusion 308 projecting from the distal end 301 of the cannula 120. In some non-limiting aspects, the protrusion 308 and the cannula 120 may be formed from a single piece of a material.

In some aspects, as shown in FIGS. 3A-C, the insertion tool 100 may further comprise a sleeve 310 at least partially around the cannula 120. In some aspects, the sleeve 310 may enclose the opening 303 at the distal end 301 of the cannula 120. In some aspects, the sleeve 310 may define an aperture 320 proximate to the distal end 301 of the cannula 120. In some aspects, the dissector tip 125 (e.g., the protrusion 308) may project through the aperture 320 of the sleeve 310.

In some aspects, the sleeve 310 may comprise a one or more perforations 312 (e.g., one or more weakened portions or score lines) defining two or more portions 314 of the sleeve 310 proximate to the distal end 301 of the cannula 120. In some aspects, the one or more perforations 312 may be separated by pulling the sleeve 310 toward the handle 110. In some aspects, as shown in FIG. 3C, the separation at the one or more perforations 312 may result in the portions 314 separating from one another. In some aspects, the separated portions 314 of the sleeve 310 may expose the opening 303 into the passage 302 of the cannula 120.

In some aspects, the sleeve 310 may be operatively connected to the actuator 140. In some aspects, the sleeve 310 may be configured to be pulled along the cannula 120 from a closed position to an open position by sliding the slider knob 144 along the track 142 in a direction away from the cannula 120. In some aspects, as shown in FIGS. 3A and 3B, when the sleeve 310 is in at the closed position, the sleeve 310 may enclose the opening 303 of the distal end 201 of the cannula 120. In some aspects, as shown in FIG. 3C, when the sleeve 310 is moved to the open position, the sleeve 310 may be pulled toward the handle 110. In some aspects, as shown in FIG. 3C, moving the sleeve 310 to the open position may separate the one or more perforations 312 and expose the opening 203 of the distal end 301 of the cannula 120. In some aspects, when the sleeve 310 is pulled toward the handle 110 of the insertion tool 100, the distal end 301 of the cannula 120 and the device 106 may apply a sufficient amount of force against the aperture 320 of the sleeve 310 to separate the portions 314 of the sleeve 310. In some aspects, the sleeve 310 may be configured to be moved toward the open position before the cannula 120 moves toward the retracted position to ensure that the distal end 301 of the cannula 120 engages the portions 314 of the sleeve 310 with a sufficient amount of force to separate or rupture the perforations or weakened portions 312.

In some aspects, as shown in FIG. 3A, the rod 130 may be disposed, at least partially, in the passage 302 of the cannula 120. In some aspects, the rod 130 may be configured remain stationary (relative to the handle 110) in the passage 302 of the cannula 120 as the cannula 120 moves between the retracted position and the extended position. In some aspects, as shown in FIG. 3A, when the cannula 120 is set at the extended position, the rod 130 may be spatially separated from the distal end 301 of the cannula 120 such that the passage 302 of the cannula 120 may receive and/or hold the device 106 proximate to the distal end 301. In some aspects, when the cannula 120 is set at the retracted position, the rod 130 may be disposed proximate the distal end 301 of the cannula 120 or protruding through the distal end 301 of the cannula 120. In some aspects, moving the cannula from the extended position to the retracted position may force the device 106 through the opening 303 of the distal end 301 of the cannula 120.

In some aspects, the insertion tool 100 may create the subcutaneous pocket 102 below the skin surface 104 by inserting the cannula 120 at the extended position with the dissector tip 125 through incision in the skin surface. In some aspects, when the dissector tip 125 and the cannula 120 are inserted into the incision and while the dissector tip 125 and the cannula 120 create the subcutaneous pocket 102, the sleeve 310 may be set at the closed position (as shown in FIGS. 3A and 3B) and may enclose the device 106 in the passage 302 of the cannula 120. In some aspects, after creating the subcutaneous pocket 102, the sleeve 310 may be moved (e.g., by pulling the sleeve 310 toward the handle 110) to the open position in which the opening 303 at the distal end 301 of the cannula 120 is exposed (as shown in FIG. 3C). In some aspects, after the moving the sleeve 310 to the open position, the insertion tool 100 may implant the device 106 by moving the cannula 120 from the extended position toward the retracted position. In some aspects, as the cannula 120 moves toward the retracted position, the rod 130 may abut against the device 106, act as a backstop, and propel the device 106 through the opening 303 at the distal end 301 of the cannula 120, at least partially out of the cannula 120, and at least partially into the subcutaneous pocket 102. In some aspects, after moving the cannula 120 to the retracted position (or simultaneously therewith), the handle 110 may be used to pull the cannula 120 out of the subcutaneous pocket 102, and the device 106 may be left in the subcutaneous pocket 102.

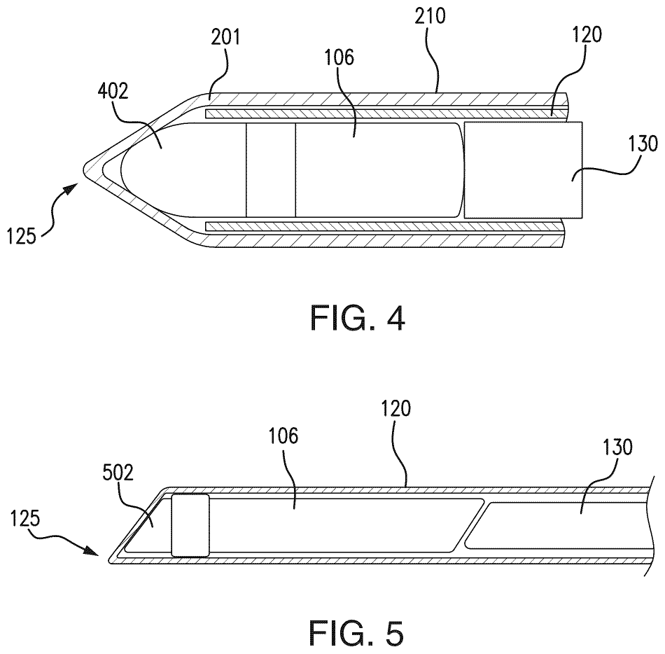

In some aspects, as shown in FIGS. 4 and 5, the device 106 may support the dissector tip 125 (e.g., a portion of the surface-area of the device 106 may contact the dissector tip 125) when the cannula 120 is at the extended position. In some aspects, the device 106 may support the dissector tip 125 when the dissector tip 125 and cannula 120 at the extended position are inserted into the incision in the skin surface 104 and create the subcutaneous pocket 102. In some aspects, as shown in FIG. 4, a front end 402 of the device 106 may support a sleeve 201 when the sleeve 201 is in a closed position in which the sleeve 201 encloses the opening at the distal end of the cannula 120. In some non-limiting aspects, as shown in FIG. 4, the device 106 may have a round edge that approximates the shape of the sleeve 201 of the dissector tip 125 that covers a blunt-shaped opening at the distal end of the cannula 210. In some alternative aspects, as shown in FIG. 5, a front end 502 of the device 106 may support a sleeve when the sleeve is in a closed position in which the sleeve encloses the opening at the distal end of the cannula 120. In some non-limiting aspects, as shown in FIG. 5, the device 106 may have a beveled edge that approximates a bevel-shape of the dissector tip 125.

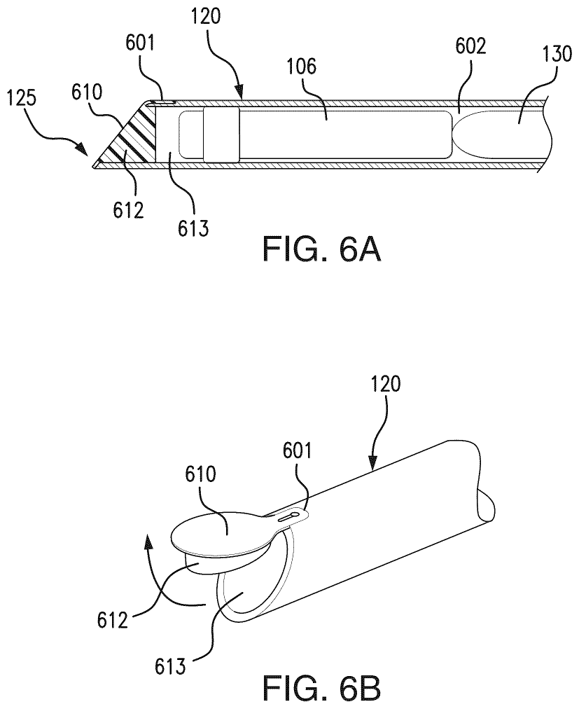

Referring to FIGS. 6A and 6B, in some aspects, the cannula 120 may include a bevel-shaped distal end 601. In some aspects, the bevel-shaped distal end 601 may define an opening 613 into the passage 602 of the cannula 120. In some non-limiting aspects, the cannula 120 may be operatively connected to the actuator 140. In some aspects, the cannula 120 may be configured to be moved from the extended position to the retracted position by sliding the slider knob 144 along the track 142 in a direction away from the cannula 120.

In some aspects, the dissector tip 125 may include a flap 610 coupled to the cannula 120. In some aspects, the flap 610 may pivot between a closed position and an open position. In some aspects, when in a closed position, the flap 610 may abut against the distal end 601 of the cannula 120 and enclose the opening 613 at the distal end 601 of the cannula 120. In some aspects, when in an open position, the flap 610 may be spatially separated from the distal end 601 and expose the opening 613 at the distal end 601 of the cannula 120. In some aspects, the flap 610 may be configured to pivot from the closed position to the open position upon the application of force, by an object, such as the device 106, moving in an axial direction against the flap 610.

In some aspects, as shown in FIGS. 6A and 6B, the flap 610 may include an extension 612 projecting from an interior surface of the flap 610. In some aspects, when the flap 610 is in the closed position, the extension 612 may engage the interior surface of the cannula 120 to ensure that the flap 610 encloses the opening 613 of the distal end 601.

In some aspects, as shown in FIG. 6A, the rod 130 may be disposed, at least partially, in the passage 602 of the cannula 120. In some aspects, the rod 130 may be configured to remain stationary (relative to the handle 110) in the passage 613 of the cannula 120 as the cannula 120 moves between the retracted position and the extended position In some aspects, as shown in FIG. 6A, when the cannula 120 is set at the extended position, the rod 130 may be spatially separated from the distal end 601 of the cannula 120 such that the passage 602 of the cannula 120 may receive and/or hold the device 106 proximate to the distal end 601. In some aspects, when the cannula 120 is set at the retracted position, the rod 130 may be disposed proximate the distal end 601 or protruding through the distal end 601 of the cannula 120. In some aspects, when the cannula 120 moves from the extended position to the retracted position, the rod 130 may force the device 106 through the opening 613 of the distal end 601 of the cannula 120.

In some aspects, the insertion tool 100 may create the subcutaneous pocket 102 below the skin surface 104 by inserting the cannula 120 at the extended position with the dissector tip 125 through an incision in the skin surface. In some aspects, when the dissector tip 125 and the cannula 120 are inserted into the incision and while the dissector tip 125 and cannula 120 create the subcutaneous pocket 102, the flap 610 may be in the closed position (as shown in FIG. 6A). In the closed position, the flap 610 may enclose the device 106 in the passage 602 of the cannula 120. In some aspects, after creating the subcutaneous pocket 102, the insertion tool 100 may implant the device 106 by moving the cannula 120 toward the retracted position. In some aspects, as the cannula 120 moves toward the retracted position, the rod 130 may abut against the device 106 to force the device 106 against the flap 610. In some aspects, forcing the device 106 against the flap 610 may cause the flap 610 to pivot from the closed position to the open position (as shown in FIG. 6B). In some aspects, the device 106 may then be pushed through the opening 613 at the distal end 601 of the cannula 120, at least partially out of the cannula 120, and at least partially into the subcutaneous pocket 102. In some aspects, after moving the cannula 120 to the retracted position (or simultaneously therewith), the handle 110 may be used to pull the cannula 120 out of the subcutaneous pocket 102, and the device 106 may be left in the subcutaneous pocket 102.

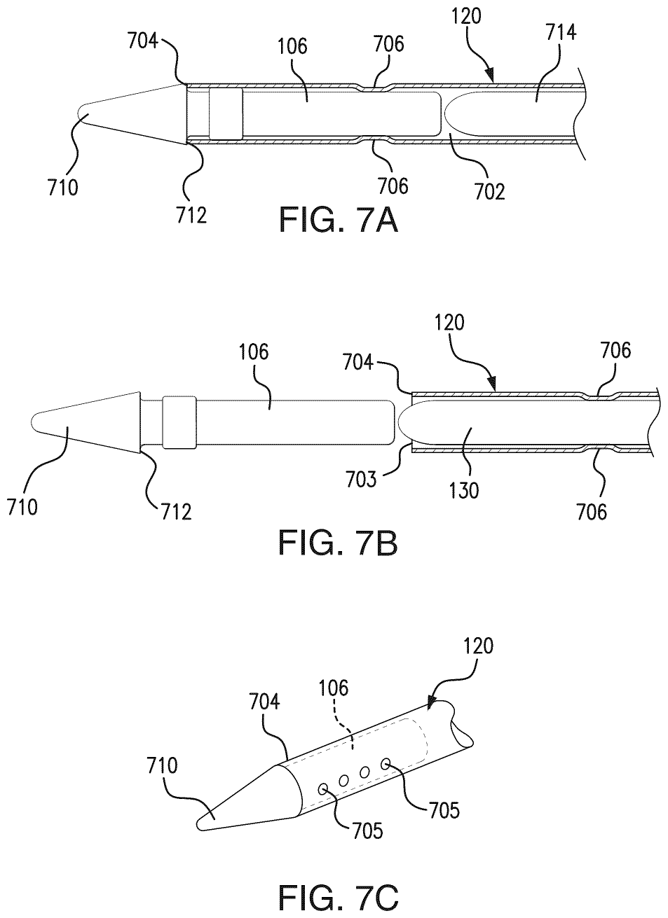

In some aspects, as shown in FIGS. 7A-7C, the cannula 120 may define a passage 702 extending along a longitudinal axis thereof. In some non-limiting aspects, the cannula 120 is operatively connected to the actuator 140. In some aspects, the cannula 120 may be configured to be moved from the extended position to the retracted position by sliding the slider knob 144 along the track 142 in a direction away from the cannula 120. In some aspects, the cannula 120 may include a distal end 704 defining an opening 703 into the passage 702. In some aspects, when set at the extended position, the cannula 120 may be configured to retain and hold at least a portion of the device 106 in the passage 702 proximate to the open distal end 704. In some aspects, the cannula 120 may comprise one or more flexible fingers 706 disposed along the cannula 120. In some aspects, the one or more flexible fingers 706 may be configured to retain the device 106 by flexing toward the longitudinal axis of the cannula 120. In some aspects, the flexible fingers 706 may be defined by a pair of parallel slits cut along the cannula 120, and each flexible finger 706 may include a strip of the dissector rod 120 defined by the pair of slits.

In some aspects, as shown in FIGS. 7A-7C, the dissector tip 125 may be integrally connected to the device 106. In some aspects, the dissector tip 125 may be a cone-shaped projection 710 disposed at a front end of the device 106. In some aspects, when the device 106 is partially retained in the passage 702 of the cannula 120, the projection 710 of the device 106 protrudes out of the distal end 704 of the dissector rod 120. In some aspects, projection 710 may include one or more shoulders 712 projecting from a side of the device 106 in a radial direction. In some aspects, when the device 106 may be retained in the passage 702 of the cannula 120, the distal end 702 of the cannula 120 may abut against the shoulders 712, thereby ensuring that the projection 710 extends away from the distal end 704 by a predetermined length.

In some aspects, as shown in FIGS. 7A and 7B, the rod 130 may be disposed, at least partially, in the passage 702 of the cannula 120. In some aspects, the rod 130 may be configured to remain stationary (relative to the handle 110) in the passage 702 of the cannula 120 as the cannula 120 moves between the retracted position and the extended position. In some aspects, as shown in FIG. 7A, when the cannula 120 is set at the extended position, the rod 130 may be spatially separated from the distal end 704 of the cannula 120 such that the passage 702 of the cannula 120 may receive at least a portion of the device 106 proximate to the distal end 704. In some aspects, when the cannula 120 is set at the retracted position, the rod 130 may be disposed proximate the distal end 704 or protruding through the distal end 704 of the cannula 120. In some aspects, when the cannula 120 is moved from the extended position to the retracted position, the rod 130 may force the device 106 through the opening 703 at the distal end 704 of the cannula 120 and at least partially out of the cannula 120.

In some aspects, as shown in FIG. 7C, the cannula 120 may further comprise one or more ports 705 for introducing a hydration fluid (e.g., saline fluid) into the passage 702. In some aspects, the hydration fluid may be introduced into the one or more ports 705 as device 106 is held in the passage 702 of the cannula 120. In some aspects, the hydration fluid may hydrate (or at least begin hydration of) at least a portion of the device 106 before implantation of the device 106. For example, for a non-limiting aspect of a device 106 that is an analyte sensor, the analyte sensor may include an analyte indicator including a hydrogel, and the hydration fluid may hydrate (or at least begin hydration of) at least the analyte indicator of the sensor before insertion. In some non-limiting aspects, the device 106 may require hydration before the device 106 can operate normally. Accordingly, in some aspects, hydration fluid in the cannula 120 may reduce or eliminate an amount of time that the device 106 is required to be implanted in the body before device 106 operates normally.

In some aspects, the insertion tool 100 may create the subcutaneous pocket 102 below the skin surface 104 by inserting the cannula 120 at the extended position with the dissector tip 125 through an incision in the skin surface. In some aspects, when the cannula 120 is inserted into the incision and while the projection 710 and the cannula 120 create the subcutaneous pocket 102, the device 106 may be partially held in the passage 702 of the cannula 120 with the projection 710 protruding away from the distal end 704 of the cannula 120. In some aspects, after creating the subcutaneous pocket 102, the insertion tool 100 may implant the device 106 by moving the cannula 120 toward the retracted position. In some aspects, as the cannula 120 moves toward the retracted position, the rod 130 may abut against the device 106, act as a backstop, and propel the device 106 through the opening 703 at the distal end 701 of the cannula 120, at least partially out of the cannula 120, and at least partially into the subcutaneous pocket 102 (as shown in FIG. 7B). In some aspects, after moving the cannula 120 to the retracted position (or simultaneously therewith), the handle 110 may be used to pull the cannula 120 out of the subcutaneous pocket 102, and the device 106 may be left in the subcutaneous pocket 102.

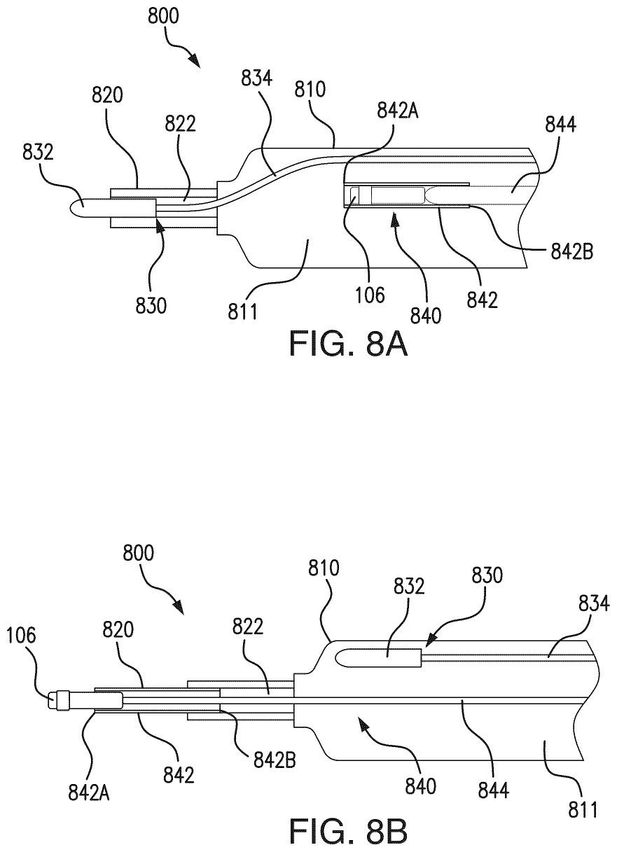

FIGS. 8A and 8B illustrate an exemplary insertion tool 800 for creating the subcutaneous pocket 102 below the skin surface 104 and implanting the device 106 in the subcutaneous pocket 102. In some aspects, the insertion tool 800 may include a handle 810, a tunneling tube 820, a dissector 830, an inserter 840, and one or more actuators (not shown). In some aspects, the handle 810 may define a cavity 811 therein. In some aspects, the tunneling tube 820 may extend from a first end 812 of the handle 810 and may define a passage 822 opening into the cavity 811 of the handle 810.

In some aspects, the tunneling tube 820 may be configured to move in an axial direction between an extended position and a retracted position. In some aspects, when the tunneling tube 820 is set at the extended position, the tunneling tube 820 and the dissector 830 may be configured to create the subcutaneous pocket 102 below the skin surface 104. In some aspects, when the tunneling tube 820 is moved to the retracted position, the tunneling tube 820 and the inserter 840 may be configured to deploy the device 106 into the subcutaneous pocket 102. In some aspects, when the tunneling tube 820 is set at the extended position, a substantial portion of the tunneling tube 820 may be disposed outside the handle 110. In some aspects, when the tunneling tube 820 is set at the retracted position, a portion or all of the tunneling tube 820 may be disposed in a cavity 811 of the handle 810.

In some aspects, the dissector 830 may comprise a first rod 834 disposed at least partially in the cavity 811 of the handle 810. In some aspects, the first rod 834 may be rigid. However, this is not required, and, in some alternative aspects, the first rod 834 may be flexible. In some aspects, the first rod 834 may comprise of a polymeric material, such as nylon, polypropylene, or polyvinylchloride. In some aspects, a dissector tip 832 may be coupled to a distal end of the first rod 834. In some aspects, the dissector tip 832 and the first rod 834 may be configured to move between an extended position and a retracted position. FIGS. 8A and 8B show the dissector tip 832 and the first rod 834 in the extended and retracted positions, respectively. In some non-limiting aspects, as shown in FIG. 8A, when at the extended position, the dissector tip 832 may protrude out of a distal end of the tunneling tube 820. In some aspects, the dissector tip 832 may be configured to create the subcutaneous pocket 102 below the skin surface 104. In some aspects, as shown in FIG. 8B, when set at the retracted position, the dissector tip 832 may be disposed in the cavity 811 of the handle 810.

In some aspects, the first rod 834 may be operatively connected to an actuator, and the actuator may be configured to move the first rod 834 and the dissector tip 832 between the extended and retracted positions. In some aspects, the actuator may be configured to move the dissector tip 832 out of the tunneling tube 820 and into the cavity 811. In some aspects, the actuator may be a slider or a lever connected to the first rod 834. In some aspects, the actuator may be a spring loaded mechanism connected to the first rod 834 to advance and retract the first rod 834. Although some aspects include an actuator configured to move the first rod 834 and the dissector tip 832 between extended and retracted positions, this actuator is not required, and, in some alternative aspects, a user may move the first rod 834 and the dissector tip 832 from the extended position shown in FIG. 8A to the retracted position shown in FIG. 8B by pulling directly on the first rod 832.

In some aspects, the inserter 840 may comprise a second rod 844 disposed at least partially in the cavity 811 of the handle 810. In some aspects, the second rod 844 may be rigid. However, this is not required, and, in some alternative aspects, the second rod 844 may be flexible. In some aspects, the inserter 840 may include a cannula 842. In some aspects, the cannula 842 may include a first end 842A configured to hold and release the device 106. In some aspects, the cannula 842 may include a second end 842B coupled to a distal end of the second rod 844. In some aspects, the second rod 844 may be configured to move along the cavity 811 of the handle 810. In some aspects, the cannula 842 may be configured to move between a retracted position and an extended position. In some aspects, the cannula 842 may be configured to slide along the second rod 844 toward the retracted position, as the tunneling tube 820 moves toward the retracted position.

FIGS. 8A and 8B show the cannula 842 in the retracted and extended positions, respectively. In some aspects, as shown in FIG. 8A, when at the retracted position, the cannula 842 may be disposed in the cavity 811 of the handle 810. In some aspects, as shown in FIG. 8B, when at the extended position, the cannula 842 may be at least partially disposed in the passage 822 of the tunneling tube 820. In some aspects, the tunneling tube 820 may hold the cannula 842 in place at its extended position. In some aspects, when the cannula 842 is at its extended position and at least partially disposed in the tunneling tube 820, the tunneling tube 820 may retract in axial direction toward the handle 810, which may also slide the cannula 842 back toward the handle 810. In some aspects, as the cannula 842 retracts with the tunneling tube 820 into the cavity 811 of the handle 810, the second rod 844 may remain in an extended position and abut against the device 106 to force the device 106 at least partially out of the cannula 842 and the tunneling tube 820 and at least partially into the subcutaneous pocket 102.

In some aspects, the second rod 844 may be operatively connected to an actuator, and the actuator may be configured to move the second rod 844, the cannula 842, and the tunneling tube 820 between retracted and extended positions. In some aspects, the actuator configured to move the second rod 844, the cannula 842, and the tunneling tube 820 between retracted and extended positions may be the same actuator that is configured to move the first rod 834 and the dissector tip 832 between retracted and extended positions. In some aspects, the actuator may be a slider knob (not shown) configured to slide along a track (not shown) disposed along the handle 810. In some aspects, the actuator may include a fork (not shown) extending from the slider knob and comprising a plurality of prongs connected to the first rod 834 and the second rod 844. In some aspects, the actuator may include a catch mechanism (not shown), such as a strut, to hold the second rod 844 in the extended position, while the actuator moves the tunneling tube 820 and the cannula 842 toward the retracted position. In some alternative aspects, a first actuator may be configured to move the second rod 844 and the cannula 842 between retracted and extended positions, and a different, second actuator may be configured to move the first rod 834 and the dissector tip 832 between retracted and extended positions.

In some aspects, the insertion tool 800 may create the subcutaneous pocket 102 below the skin surface 104 by setting the dissector 830 in the extended position (as shown in FIG. 8A) and inserting the tunneling tube 820 with the dissector tip 832 protruding from the tunneling tube 820 through an incision in the skin surface. In some aspects, after creating the subcutaneous pocket 102, the dissector tip 832 and first rod 834 may be moved from their extended position to their retracted position. In some aspects, the second rod 844 and cannula 842 may then be moved from the retracted position toward the extended position so that the cannula 842 (with the device 106 held therein) is disposed at least partially in the tunneling tube 820. In some aspects, with the cannula 842 disposed at least partially in the tunneling tube 820, the tunneling tube 820 may be retracted toward the handle 810 to slide the cannula 842 along the second rod 844 and at least partially into the cavity 811 of the handle 810. In some aspects, as the cannula 842 retracts with the tunneling tube 820 into the cavity 811 of the handle 810, the second rod 844 may remain in the extended position (e.g., using the catch mechanism of the actuator 950) and abut against the device 106 to force the device 106 at least partially out of the cannula 842 and the tunneling tube 820. In some aspects, the handle 810 may be used to pull the tunneling tube 820 out of the subcutaneous pocket 102, and the device 106 may be left in the subcutaneous pocket 102. In some aspects, one or more of the dissector tip 832, first rod 834, cannula 842, and second rod 844 may be moved while keeping the tunnel tube 820 disposed in the subcutaneous pocket 102.

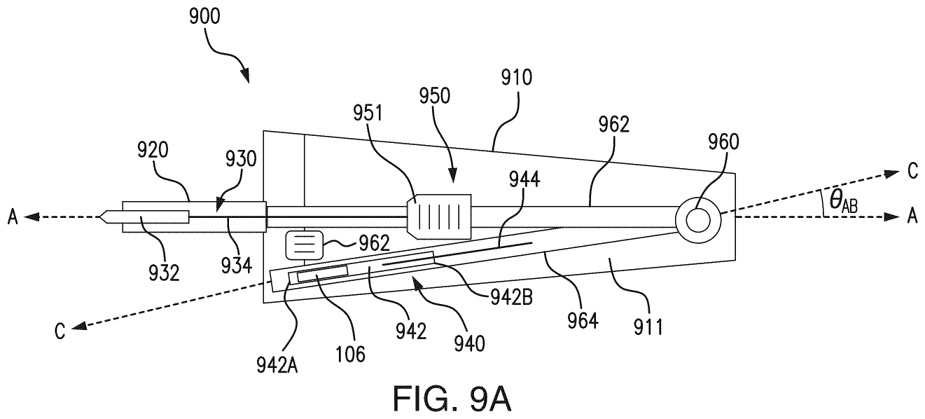

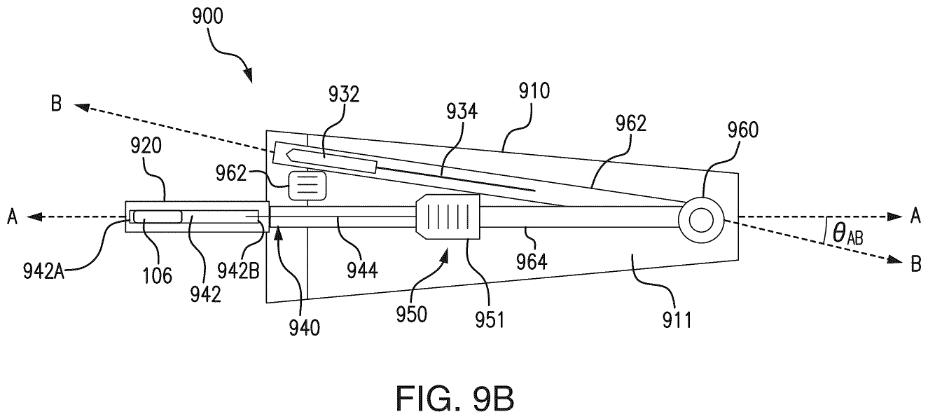

FIGS. 9A and 9B illustrate an exemplary insertion tool 900 for creating the subcutaneous pocket 102 below the skin surface 104 and implanting the device 106 in the subcutaneous pocket 102. In some aspects, the insertion tool 900 comprises a handle 910, a tunneling tube 920, a dissector 930, an inserter 940, and an actuator 950.

In some aspects, the insertion tool 900 may include one or more of the same features as the aspect of the insertion tool 800 shown in FIGS. 8A and 8B. For example, the tunneling tube 920 may be configured to move between an extended position to create the subcutaneous pocket 102 using the dissector 930 and a retracted position to deploy the device 106 into the subcutaneous pocket 102 using the inserter 940. In some aspects, the dissector 930 may include a first rod 934 configured to move along a cavity 911 of the handle 910 and a dissector tip 932 coupled to a distal end of the first rod 934. In some aspects, the inserter 940 may include a second rod 944 configured to move along the cavity 911 of the handle 910 and a cannula 942 configured to hold the device 106 at a first end 942A and coupled to the second rod 944 at a second end 942B.

In some aspects, as shown in FIGS. 9A and 9B, the dissector 930 may be configured to pivot from an operating position disposed along a first axis A defined by the tunneling tube 920 to an idle position disposed along a second axis B. In some aspects, the second axis B may extend at an acute angle θ_{AB} (e.g., 10°) with respect to the first axis A. In some aspects, the inserter 940 may be configured to pivot from an idle position disposed along a third axis C to an operating position disposed along the first axis A. In some aspects, the third axis C may extend at an acute angle θ_{AC} (e.g., 10°) with respect to the first axis A.

In some aspects, as shown in FIGS. 9A and 9B, the insertion tool 900 may further comprise a hinge 960. In some non-limiting aspects, the hinge 960 may be disposed in the handle 910. In some aspects, the hinge 960 may include a first arm 962 disposed in the cavity 911 and configured to pivot about an axis defined by the hinge 960 from the operating position extending along the first axis A to the idle position extending along the second axis B. In some aspects, the hinge 960 may include a second arm 964 disposed in the cavity 911 and configured to pivot about an axis defined by the hinge 960 from the idle position extending along the third axis C to the operating position extending along the first axis A.

In some aspects, the first arm 962 may define a passage, and the dissector tip 932 and the first rod 934 may be disposed in the passage of the first arm 962. In some aspects, the dissector 930 may be configured to move between the operating and idle positions as the first arm 962 pivots about the hinge 960. In some aspects, the dissector tip 932 and the first rod 930 may be configured to move along the passage of the first arm 962 between retracted and extended positions when the first arm 962 is set at the operating position extending along the first axis A. In some aspects, the dissector tip 932 and the first rod 930 may be configured to remain stationary in the retracted position when the first arm 962 is set at the idle position extending along the second axis B.

In some aspects, the second arm 964 may define a passage, and the cannula 942 and the second rod 944 may be disposed in the passage of the second arm 964 such that the inserter 940 may be configured to move between the idle and operating positions as the second arm 964 pivots about the hinge 960. In some aspects, the cannula tube 942 and the second rod 944 may be configured to move along the passage of the second arm 964 is set at the operating position extending along the first axis A. In some aspects, the cannula tube 942 and the second rod 944 may be configured to remain stationary in the retracted position when the second arm 964 is set at the idle position extending along the third axis C.

In some aspects, the insertion tool 900 may include a pivot actuator 962 operatively connected to the dissector 930 and the inserter 940 to cause the pivoting movement of the dissector 930 and the inserter 940 between the idle and operating positions. In some aspects, the pivot actuator 962 may include, for example and without limitation, a twist knob or a slider. In other aspects (not shown), the insertion tool 900 may comprise other internal mechanisms (e.g. a spring-loaded device) to move the dissector 930 and the inserter 940 between the idle and operating positions.

In some aspects, the actuator 950 may be disposed in the handle 910 and may be operatively connected to the tunneling tube 920, the dissector 930, and the inserter 940. In some aspects, the actuator 950 may be configured to selectively force the tunneling tube 920, the dissector 930, and the inserter 940 to move between retracted and extended positions. In some aspects, the actuator 950 may include a track (not shown) extending along the handle 910 and a slider knob 951 configured to slide along the track. In some aspects, the actuator 950 may include a fork (not shown) extending from the slider knob 951 and configured to removably connect to the dissector 930 and inserter 940 when pivoted into the operating position. In some aspects, the actuator 950 may include a catch mechanism (not shown), such as a strut, to hold the second rod 944 in the extended position, while the actuator 950 moves the tunneling tube 920 and the cannula 942 toward the retracted position.

In some aspects, the insertion tool 900 may create the subcutaneous pocket 102 below the skin surface 104 by first setting the dissector 930 in the operating position (e.g., using the pivot actuator 962) and sliding the dissector tip 932 and the first rod 934 to the extended position (as shown in FIG. 9A) (e.g., using the actuator 950). In some aspects, the subcutaneous pocket 102 may be created by inserting the tunneling tube 920 with the dissector tip 932 protruding from the tunneling tube 920 through an incision in the skin surface. In some aspects, after creating the subcutaneous pocket 102, the dissector tip 932 and first rod 934 may be moved from their extended position to their retracted position. In some aspects, the dissector 930 in the retracted position may be pivoted from the operating position along the first axis A to the idle position along the second axis B, and the inserter 940 may be pivoted from the idle position along the third axis C to the operating position along the first axis A (e.g., using pivot actuator 962). In some aspects, the second rod 944 and cannula 942 of the inserter 940 in the operating position may then be moved from the retracted position toward the extended position (e.g., using the actuator 950) so that the cannula 942 (with the device 106 held therein) is disposed at least partially in the tunneling tube 920. In some aspects, with the cannula 942 disposed at least partially in the tunneling tube 920, the tunneling tube 920 may be retracted toward the handle 910 (e.g., using the actuator 950) to slide the cannula 942 along the second rod 944 and into at least partially into the cavity 911 of the handle 910. In some aspects, as the cannula 942 retracts with the tunneling tube 920 into the cavity 911 of the handle 910, the second rod 944 may remain in the extended position (e.g., using the catch mechanism of the actuator 950), abuts against the device 106, act as a backstop, and force the device 106 at least partially out of the cannula 942 and the tunneling tube 920. In some aspects, after at least partially retracting the tunneling tube 920 into the cavity 911 of the handle 910 (or simultaneously therewith), the handle 910 may be used to pull the tunneling tube 920 out of the subcutaneous pocket 102, and the device 106 may be left in the subcutaneous pocket 102. In some aspects, one or more of the dissector tip 932, first rod 934, cannula 942, second rod 944, actuator 950, and pivot actuator 962 may be moved while keeping the tunneling tube 920 disposed in the subcutaneous pocket 102.

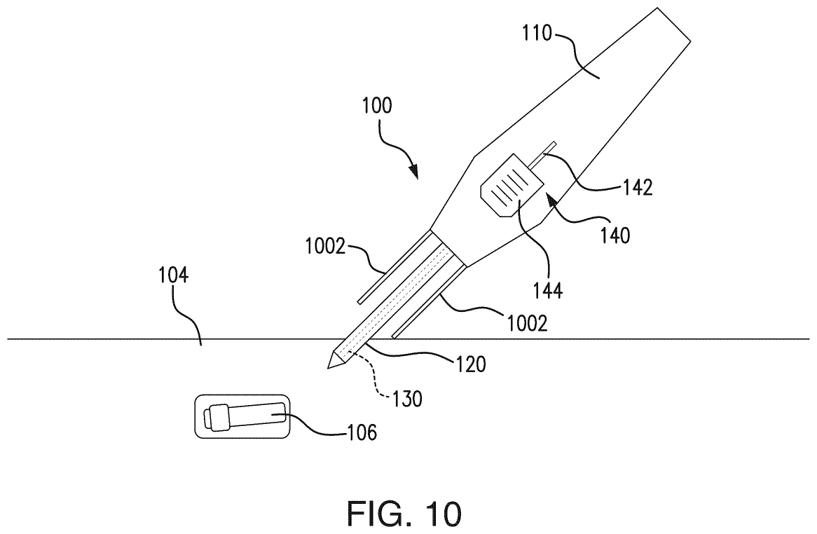

FIG. 10 illustrates an exemplary insertion tool 1000, which may comprise any of the features described in FIGS. 1-7. In some aspects, the insertion tool 1000 may include one or more guide prongs 1002 extending from a first end of the handle 110. In some aspects, the one or more guide prongs 1002 may be configured to limit the depth at which the dissector rod 120 is capable of creating the subcutaneous pocket 102. In some aspects, the dissector rod 120 may extend farther from the end of the handle 110 than the one or more guide prongs 1002.

In some aspects, all the configurations of the insertion tools 100, 800, 900, and 1000 described herein may include a loading port disposed in the handle and in communication with the cannula for introducing a hydration fluid (e.g., saline fluid) into the cannula. In some aspects, the hydration fluid may hydrate (or at least begin hydration of) at least a portion of the device 106 (e.g., an analyte indicator or hydrogel of the device 106) before implantation of the device 106.

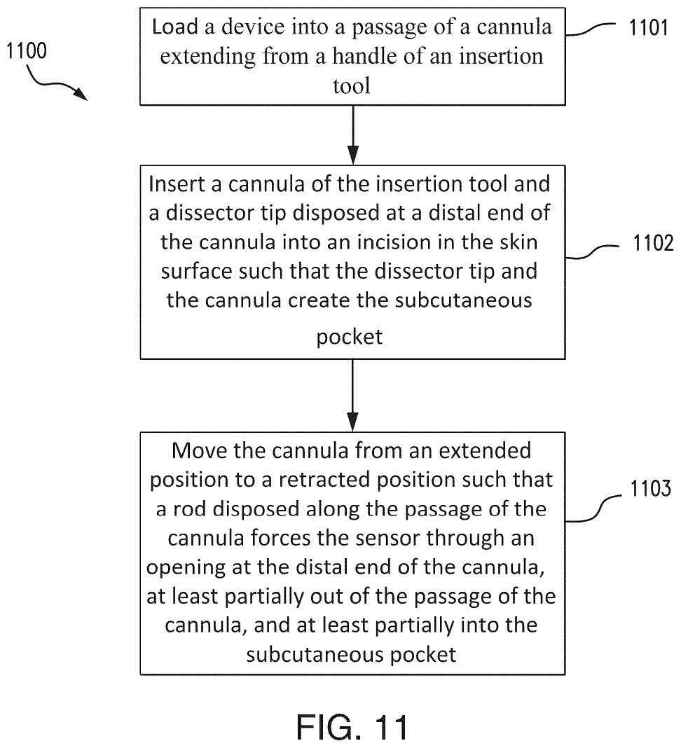

FIG. 11 is a flow chart showing a method 1100 of creating a subcutaneous pocket below a skin surface and implanting a device in the subcutaneous pocket embodying aspects of the present disclosure. In some aspects, the insertion tools 100, 800, 900, and 1000 described above in FIGS. 1-10 may be used to create the subcutaneous pocket and implant the device according to the method described in FIG. 11.

In some aspects, the method 1100 may include a step 1101 of loading the device into a passage of a cannula extending from a handle of the insertion tool.

In some aspects, the method 1100 may include a step 1102 of inserting a cannula of the insertion tool and a dissector tip disposed at a distal end of the cannula into an incision in the skin surface such that the dissector tip and the cannula create the subcutaneous pocket.

In some aspects, the method 1100 may include a step 1103 of moving the cannula from an extended position to a retracted position such that a rod disposed along the passage of the cannula forces the device through an opening at the distal end of the cannula, out of the passage of the cannula, and into the subcutaneous pocket.

In some aspects, the method 1100 may further include, after loading the device into the passage and before inserting the cannula and the dissector tip, a step of pulling a sleeve around the distal end of the cannula to enclose the opening in the distal end of the cannula. In some aspects, the method 1100 may further include, before the step of loading, a step of pulling a sleeve received around the cannula toward the handle to expose the opening in the distal end of the cannula.

In some aspects, the method 1100 may further include using an actuator disposed in the handle to move the cannula from the extended position to the retracted position. In some aspects the method 1100 may further include pulling the cannula away from the subcutaneous pocket.

In some aspects, the method 110 may further include a step of pulling a sleeve along the cannula from a closed position, in which the sleeve encloses the opening at the distal end of the cannula, to an open position, in which the sleeve exposes the opening at the distal end of the cannula.

In some aspects, the insertion tool may further include a flap covering the opening at the distal end of the cannula. In some aspects, the method 1100 may further include a step of moving the cannula from the extended position to the retracted position spatially separates the flap from the distal end of the cannula and exposes the opening at the distal end of the cannula.

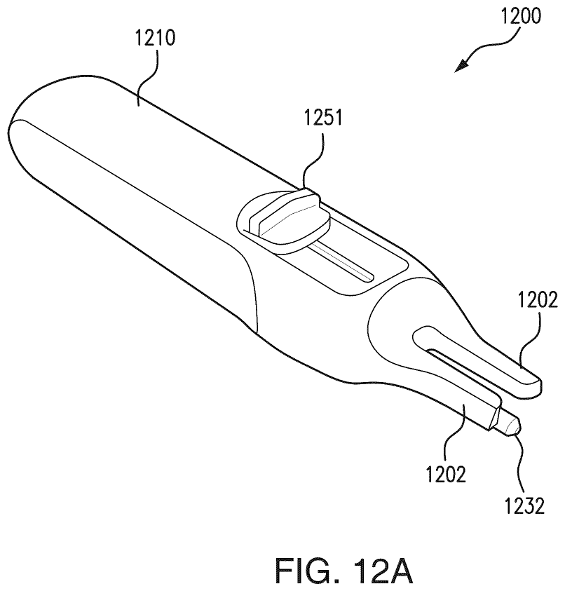

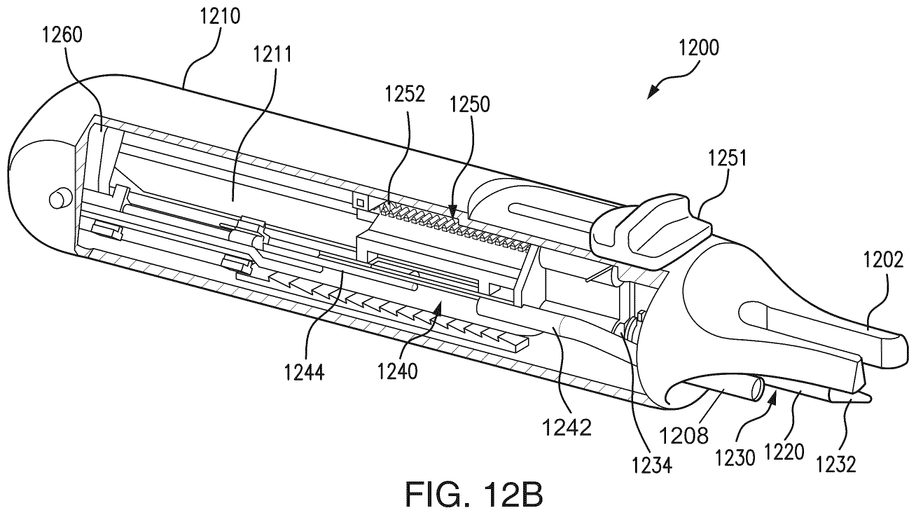

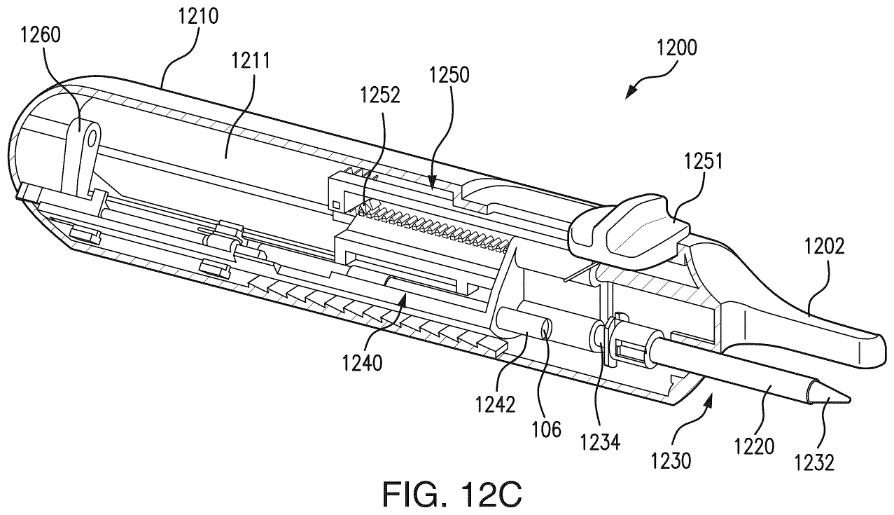

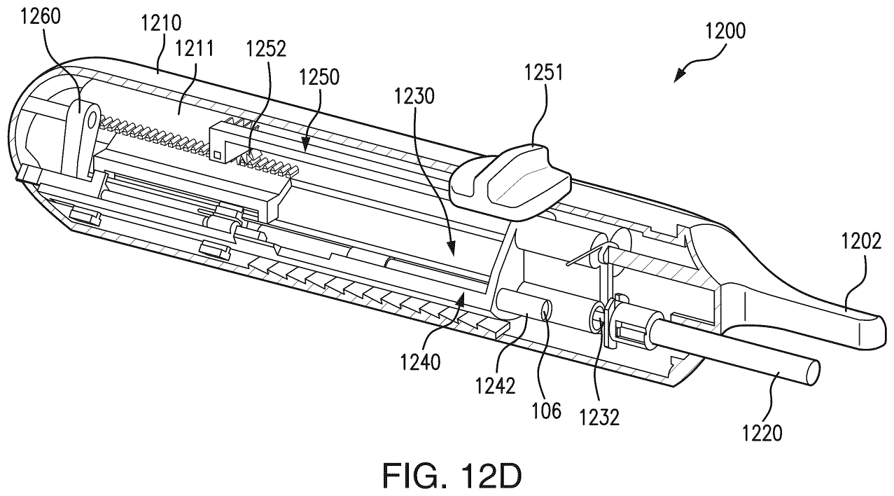

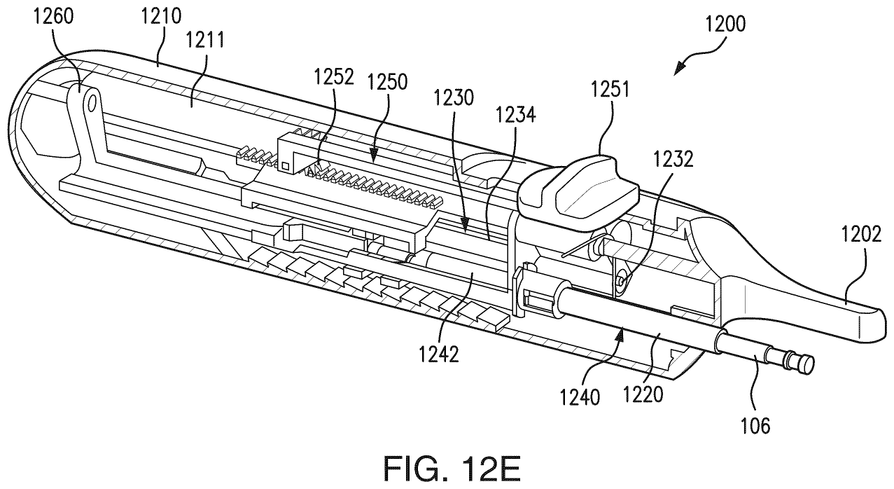

FIGS. 12A-12E illustrate an exemplary insertion tool 1200 for creating the subcutaneous pocket 102 below the skin surface 104 and implanting the device 106 in the subcutaneous pocket 102. In some aspects, the insertion tool 1200 comprises a handle 1210, a tunneling tube 1220, a dissector 1230, an inserter 1240, and an actuator 1250.

In some aspects, the insertion tool 1200 may include one or more of the same features as the aspects of the insertion tools 800 and 900 shown in FIGS. 8A-9B. For example, the tunneling tube 1220 may be configured to move between an extended position to create the subcutaneous pocket 102 using the dissector 1230 and a retracted position to deploy the device 106 into the subcutaneous pocket 102 using the inserter 1240. In some aspects, the dissector 1230 may include a first rod 1234 configured to move along a cavity 1211 of the handle 1210 and a dissector tip 1232 coupled to a distal end of the first rod 1234. In some aspects, the inserter 1240 may include a second rod 1244 configured to move along the cavity 1211 of the handle 1210 and a cannula 1242 configured to hold the device 106 at a first end and be coupled to the second rod 1244 at a second end.

In some aspects, the dissector 1230 may be configured to move from an operating position in which a longitudinal axis of the dissector 1230 coincides with a longitudinal axis of the tunneling tube 1220 to an idle position is which the longitudinal axis of the dissector 1230 does not coincide with (e.g., is shifted away from and as is parallel to) the longitudinal axis of the tunneling tube 1220. In some aspects, the inserter 1240 may be configured to move from an idle position in which a longitudinal axis of the inserter 1240 does not coincide with (e.g., is shifted away from and as is parallel to) the longitudinal axis of the tunneling tube 1220 to an operating position in which the longitudinal axis of the inserter 1240 coincides with the longitudinal axis of the tunneling tube 1220.

In some aspects, the insertion tool 1200 may comprise a hinge 1260. In some non-limiting aspects, the hinge 1260 may be disposed in the handle 1210. In some aspects, the dissector 1230 and the inserter 1240 may be configured to rotate about the hinge 1260. In some aspects, the dissector 1230 may be configured to rotate from its operating position to its idle position as the inserter 1240 rotates from its idle position to its operating position.

In some aspects, the insertion tool 1200 may include a rotation actuator operatively connected to the dissector 1230 and the inserter 1240 to cause the rotation of the dissector 1230 and the inserter 1240. In some aspects, the rotation actuator may include, for example and without limitation, a spring. In some aspects, the rotation actuator may be configured to automatically rotate the dissector 1230 from its operating position to its idle position and the inserter 1240 from its idle position to its operating position when an actuator 1250 is moved to a retracted positon. In some alternative aspects, the rotation actuator may actuator may include, for example and without limitation, a twist knob or a slider, and the rotation actuator may be configured to manually rotate the dissector 1230 and inserter 1240.

In some aspects, the actuator 1250 may be disposed in the handle 1210 and may be operatively connected to one or more of the tunneling tube 1220, the dissector 1230, and the inserter 1240. In some aspects, the actuator 1250 may be configured to selectively force one or more of the tunneling tube 1220, the dissector 1230, and the inserter 1240 to move between retracted and extended positions. In some aspects, the actuator 1250 may include a track extending along the handle 1210 and a slider knob 1251 configured to slide along the track. In some aspects, the actuator 1250 may include a gear 1252 that engages with teeth. In some aspects, the gear 1252 may enable relatively small movements of the slider knob 1251 to cause relatively large movements of the dissector 1230 and/or the inserter 1240.

In some aspects, the insertion tool 1200 may create the subcutaneous pocket 102 below the skin surface 104 with the dissector 1230 in the operating position and the dissector tip 1232 and the first rod 1234 in the extended position (as shown in FIGS. 12B and 12C). In some aspects, the subcutaneous pocket 102 may be created by inserting the tunneling tube 1220 with the dissector tip 1232 protruding from the tunneling tube 1220 through an incision in the skin surface. In some aspects, after creating the subcutaneous pocket 102, the dissector tip 1232 and first rod 1234 may be moved from their extended position to their retracted position (e.g., using the actuator 1250), as shown in FIG. 12D. In some aspects, the dissector 1230 in the retracted position may be rotated from the operating position on the longitudinal axis of the tunneling tube 1220 to the idle position, and the inserter 1240 may be rotated from the idle position to the operating position on the longitudinal axis of the tunneling tube 1220. In some aspects, the second rod 1244 and cannula 1242 of the inserter 1240 in the operating position may then be moved from the retracted position toward the extended position (e.g., using the actuator 1250) so that the cannula 1242 (with the device 106 held therein) is disposed at least partially in the tunneling tube 1220. In some aspects, with the cannula 1242 disposed at least partially in the tunneling tube 1220, the tunneling tube 1220 may be retracted toward the handle 1210 (e.g., using the actuator 950) to slide the cannula 1242 along the second rod 1244 and into at least partially into the cavity 1211 of the handle 1210. In some aspects, as the cannula 1242 retracts with the tunneling tube 1220 into the cavity 1211 of the handle 1210, the second rod 1244 may remain in the extended position (e.g., using a catch mechanism of the actuator 1250), abut against the device 106, act as a backstop, and force the device 106 at least partially out of the cannula 1242 and the tunneling tube 1220 (as shown in FIG. 12E). In some aspects, the insertion tool 1200 may include a mechanism (e.g., a ratchet) that, after the inserter 1240 is rotated to the operating position, allows only forward movement (and prevents backward movement) of the slider knob 1251 of the actuator 1250 so that the second rod 1244 and cannula 1242 of the inserter 1240 may only be moved from the retracted position toward the extended position.

In some aspects, after at least partially retracting the tunneling tube 1220 into the cavity 1211 of the handle 1210 (or simultaneously therewith), the handle 1210 may be used to pull the tunneling tube 1220 out of the subcutaneous pocket 102, and the device 106 may be left in the subcutaneous pocket 102. In some aspects, one or more of the dissector tip 1232, first rod 1234, cannula 1242, second rod 1244, actuator 1250, and rotation actuator may be moved while keeping the tunneling tube 1220 disposed in the subcutaneous pocket 102.

In some aspects, as shown in FIGS. 12A-12E, the insertion tool 1200 may include one or more guide prongs 1202 extending from a first end of the handle 1210. In some aspects, the one or more guide prongs 1202 may be configured to limit the depth at which the dissector rod 1220 is capable of creating the subcutaneous pocket 102. In some aspects, the dissector rod 1220 may extend farther from the end of the handle 110 than the one or more guide prongs 1202.

In some aspects, insertion tool 1200 include a loading port disposed in the handle 1210 and in communication with the cannula 1242 for introducing a hydration fluid (e.g., saline fluid) into the cannula 1242. In some aspects, the hydration fluid may hydrate (or at least begin hydration of) at least a portion of the device 106 (e.g., an analyte indicator or hydrogel of the device 106) before implantation of the device 106. In some aspects, the cannula 1242 may act as a hydration cavity inside the tool 1200 that can be filled by tubing 1208 (e.g., luer lock tubing), which may extend into the cavity within the cannula 1242 that holds the device 106 (e.g., via the loading port). In some aspects, the cavity may be sealed off as it hydrates the device 106.

In some aspects, the blunt dissector functionality of the tool 1200 may be utilized to create the subcutaneous tunnel with the dissector tip 1232 and tunneling tube 1220 (e.g., a steel tunneling tube). In some aspects, the insertion tool 1200 may create the subcutaneous tunnel while the device 106 is hydrating. In some aspects, after the subcutaneous tunnel is created, the dissector 1230 may be retracted using the slider 1251 of the actuator 1250 and then a rotation actuator (e.g., a spring) may align the device 106 into the bore that is used for ejection. Then, by sliding the thumb slider forward, the device 106 may be deployed, and the tool 1200 may be pulled from the insertion site.

In some aspects, the insertion tool 1200 may be used as a mating component for tubing 1208 (e.g., luer lock tubing) to fill the cavity of the cannula 1242 in which the device 106 resides. In some aspects, the rotation actuator may be spring loaded feature that aligns the device 106 with the tunneling tube 1230. In some aspects, the insertion tool 1200 may include one or more depth guides 1202. In some aspects, the insertion tool 1200 may have the different sequences of use triggered off the slider knob 1251.

In some aspects, a method of creating a subcutaneous pocket 102 below a skin surface 104 and implanting a device 106 in the subcutaneous pocket 102 using the insertion tool 1200 may include a first step of using the insertion tool 1200 with the dissector tip 1232 and the first rod 1234 in the extended position to creating the subcutaneous pocket 102. In some aspects, during the first step, a tubing 1208 (e.g., luer lock tube) may seal off device 106 for hydration. In some aspects, the method may include a second step of retracting the slider knob 1251 to move the dissector 1230 to a retracted positon. In some aspects, the second step may include rotating the dissector 1230 from an operating position to an idle position and rotating the inserter 1240 from an idle positon to an operating positon. In some aspects, the method may include a third step of pushing the slider knob 1251 forward, which may push the device 106 into the subcutaneous pocket 102 created in the first step.

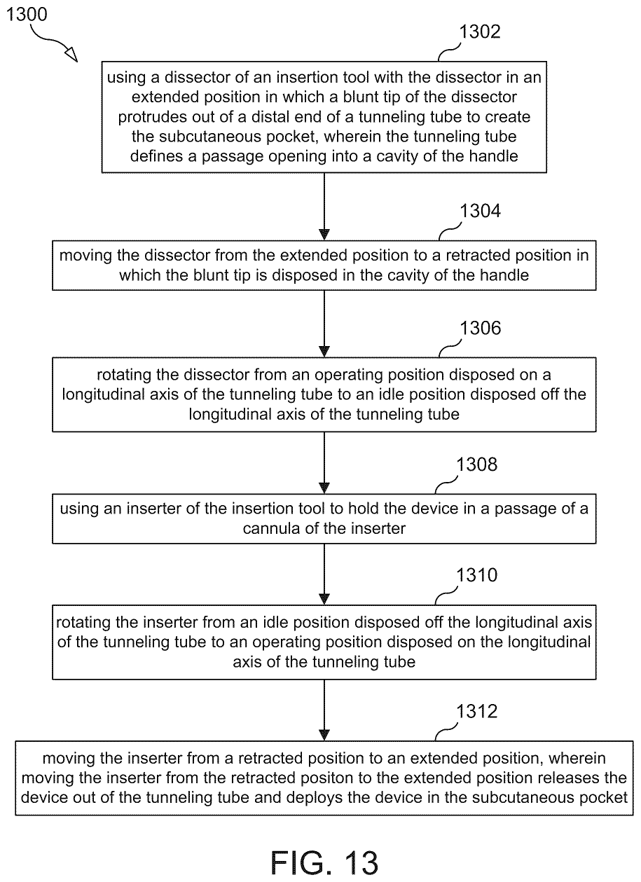

FIG. 13 is a flow chart showing a method 1300 of creating a subcutaneous pocket below a skin surface and implanting a device in the subcutaneous pocket embodying aspects of the present disclosure. In some aspects, the insertion tool 1200 described above in FIGS. 12A-12E may be used to create the subcutaneous pocket and implant the device according to the process 1300 described in FIG. 13.

In some aspects, the process 1300 may include a step 1302 of using a dissector 1230 of an insertion tool 1200 with the dissector 1230 in an extended position in which a blunt tip 1232 of the dissector 1230 protrudes out of a distal end of a tunneling tube 1220 to create the subcutaneous pocket 102. In some aspects, the tunneling tube 1220 may extend from a first end of a handle 1210 of the insertion tool 1200 and defines a passage opening into a cavity 1211 of the handle 1210.

In some aspects, the process 1300 may include a step 1304 of moving the dissector 1230 from the extended position to a retracted position in which the blunt tip 1232 is disposed in the cavity of the handle 1210.

In some aspects, the process 1300 may include a step 1306 of rotating the dissector 1230 from an operating position disposed on a longitudinal axis of the tunneling tube 1220 to an idle position disposed off the longitudinal axis of the tunneling tube 1220.

In some aspects, the process 1300 may include a step 1308 of using an inserter 1240 of the insertion tool 1200 to hold the device 106 in a passage of a cannula 1242 of the inserter 1240. In some aspects, the inserter 1240 may hold hydration fluid in the passage of the cannula 1242.

In some aspects, the process 1300 may include a step 1310 of rotating the inserter 1240 from an idle position disposed off the longitudinal axis of the tunneling tube 1220 to an operating position disposed on the longitudinal axis of the tunneling tube 1220.

In some aspects, the process 1300 may include a step 1312 of moving the inserter 1240 from a retracted position to an extended position. In some aspects, moving the inserter 1240 from the retracted positon to the extended position may release the device 106 out of the tunneling tube 1220 and deploys the device 106 in the subcutaneous pocket 102. In some aspects, the inserter 1240 may be moved from the retracted position to the extended position while the inserter 1240 is in the operating position.

In some aspects, rotating the dissector 1230 in step 1306 and/or rotating the inserter 1240 in step 1310 may include using a hinge 1260 rotatably coupled to the dissector 1230 and the inserter 1240. In some aspects, rotating the inserter 1240 in step 1310 may occur after rotating the dissector 1230 in step 1306. In some alternative aspects, rotating the dissector 1230 in step 1306 and rotating the inserter 1240 in step 1310 may occur simultaneously. In some aspects, moving the dissector 1230 from the extended position to the retracted position and moving the inserter 1240 from the retracted position to the extended position may include using a slider knob 1251 to cause the inserter 1240 and the dissector 1230 to move.

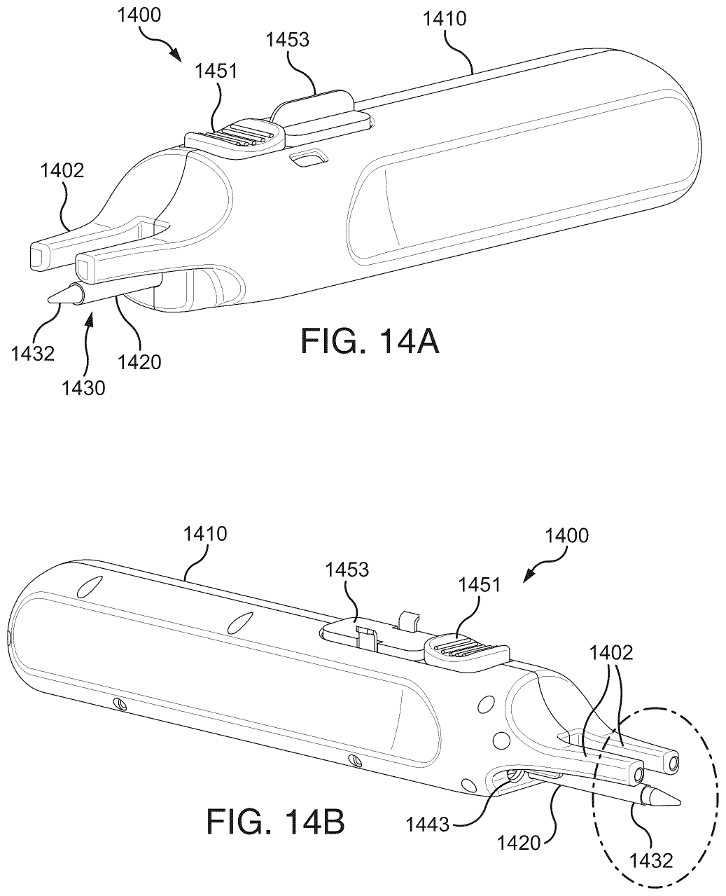

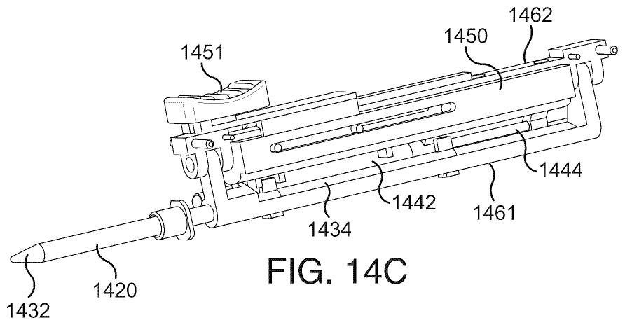

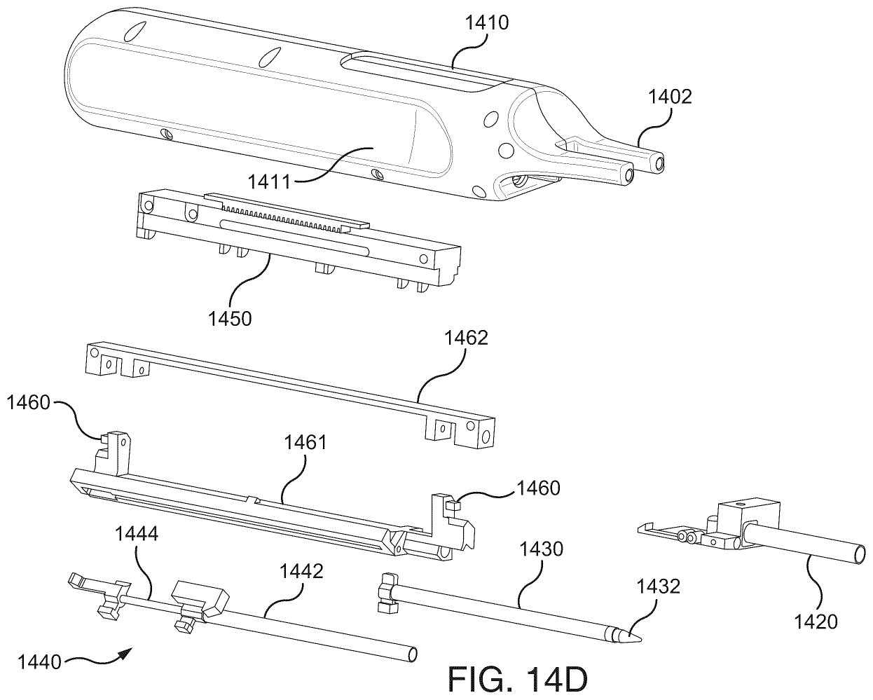

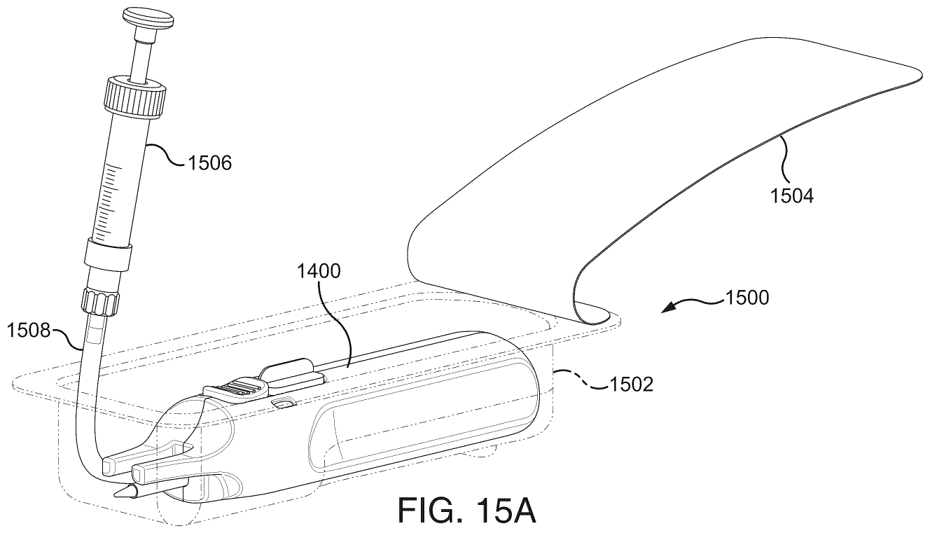

FIGS. 14A-14D illustrate an exemplary insertion tool 1400 for creating the subcutaneous pocket 102 below the skin surface 104 and implanting the device 106 in the subcutaneous pocket 102. In some aspects, the insertion tool 1400 may include a handle 1410, a tunneling tube 1420, a dissector 1430, an inserter 1440, an actuator 1450, and a stopper 1453.

In some aspects, the insertion tool 1400 may include one or more of the same features as the aspects of the insertion tool 1200 shown in FIGS. 12A-12E. For example, the tunneling tube 1420 may be configured to move between an extended position to create the subcutaneous pocket 102 using the dissector 1430 and a retracted position to deploy the device 106 into the subcutaneous pocket 102 using the inserter 1440. In some aspects, the dissector 1430 may include a first rod 1434 configured to move along a cavity 1411 of the handle 1410 and a dissector tip 1432 coupled to a distal end of the first rod 1434. In some aspects, the inserter 1440 may include a second rod 1444 configured to move along the cavity 1411 of the handle 1410 and a cannula 1442 configured to hold the device 106 at a first end and be coupled to the second rod 1444 at a second end.

In some aspects, the dissector 1430 may be configured to move from an operating position in which a longitudinal axis of the dissector 1430 coincides with a longitudinal axis of the tunneling tube 1420 to an idle position is which the longitudinal axis of the dissector 1430 does not coincide with (e.g., is shifted away from and as is parallel to) the longitudinal axis of the tunneling tube 1420. In some aspects, the inserter 1440 may be configured to move from an idle position in which a longitudinal axis of the inserter 1440 does not coincide with (e.g., is shifted away from and as is parallel to) the longitudinal axis of the tunneling tube 1420 to an operating position in which the longitudinal axis of the inserter 1440 coincides with the longitudinal axis of the tunneling tube 1420.

In some aspects, the insertion tool 1400 may include a swing 1461. In some aspects, the swing 1461 may include one or more hinges 1460. In some aspects, the swing 1461 including the one or more hinges 1460 may be disposed in the handle 1410. In some aspects, the insertion tool 1400 may include a frame 1462, and the one or more hinges 1460 of the swing 1461 may be attached rotatably to the frame 1462. In some aspects, the swing 1461 may be configured to rotate the dissector 1430 and the inserter 1440 about the one or more hinges 1460. In some aspects, the dissector 1430 may be configured to rotate from its operating position to its idle position as the inserter 1440 rotates from its idle position to its operating position.

In some aspects, the insertion tool 1400 may include a rotation actuator operatively connected to the dissector 1430 and the inserter 1440 to cause the rotation of the dissector 1430 and the inserter 1440. In some aspects, the rotation actuator may include, for example and without limitation, a spring. In some aspects, the rotation actuator may be configured to automatically rotate the dissector 1430 from its operating position to its idle position and the inserter 1440 from its idle position to its operating position when an actuator 1450 is moved to a retracted positon. In some alternative aspects, the rotation actuator may include, for example and without limitation, a twist knob or a slider, and the rotation actuator may be configured to manually rotate the dissector 1430 and inserter 1440.

In some aspects, the actuator 1450 may be disposed in the handle 1410 and may be operatively connected to one or more of the tunneling tube 1420, the dissector 1430, and the inserter 1440. In some aspects, the actuator 1450 may be configured to selectively force one or more of the tunneling tube 1420, the dissector 1430, and the inserter 1440 to move between retracted and extended positions. In some aspects, the actuator 1450 may include a linear actuator (e.g., a rack and pinion). In some aspects, the actuator 1450 may include a track extending along the handle 1410 and a slider knob 1451 configured to slide along the track. In some aspects, the actuator 1450 may include a gear that engages with teeth. In some aspects, the gear may enable relatively small movements of the slider knob 1451 to cause relatively large movements of the dissector 1430 and/or the inserter 1440.

In some aspects, the stopper 1453 of the insertion tool 1400 may be configured to prevent movement of the slider knob 1451 from a forward position and, thereby, prevent movement of the dissector tip 1432 from an extended position. In some aspects, the stopper 1453 may prevent movement (e.g., accidental movement) of the slider knob 1451 during (i) shipping/transportation of the of insertion tool 1400, (ii) sterilization of the insertion tool 1400, and/or (iii) creation of the subcutaneous pocket 102 below the skin surface 104 with the dissector tip 1432 in the extended position. In some aspects, the stopper 1453 may act as a safety feature that prevents attempted creation of the subcutaneous pocket 102 without the dissector tip 1432 at its extended position. In some aspects, the stopper 1453 may be configured to be removed from the insertion tool 1400 (e.g., after creation of the subcutaneous pocket 102 below the skin surface 104). In some aspects, after removal of the stopper 1453, the slider knob 1451 may be moved from the forward position (e.g., to move the dissector tip 1432 and first rod 1434 from their extended position to their retracted position).

FIGS. 14A and 14B show different aspects of the stopper 1453. In some aspects, as shown in FIG. 14A, the stopper 1453 may include a ridge (e.g., a tab), and a user may remove the stopper 1453 from the insertion tool 1400 by pulling on the ridge. In some alternative aspects, as shown in FIG. 14B, the stopper 1453 may include one or more clips, and a user may remove the stopper 1453 from the insertion tool 1400 by squeezing the one or more clips.

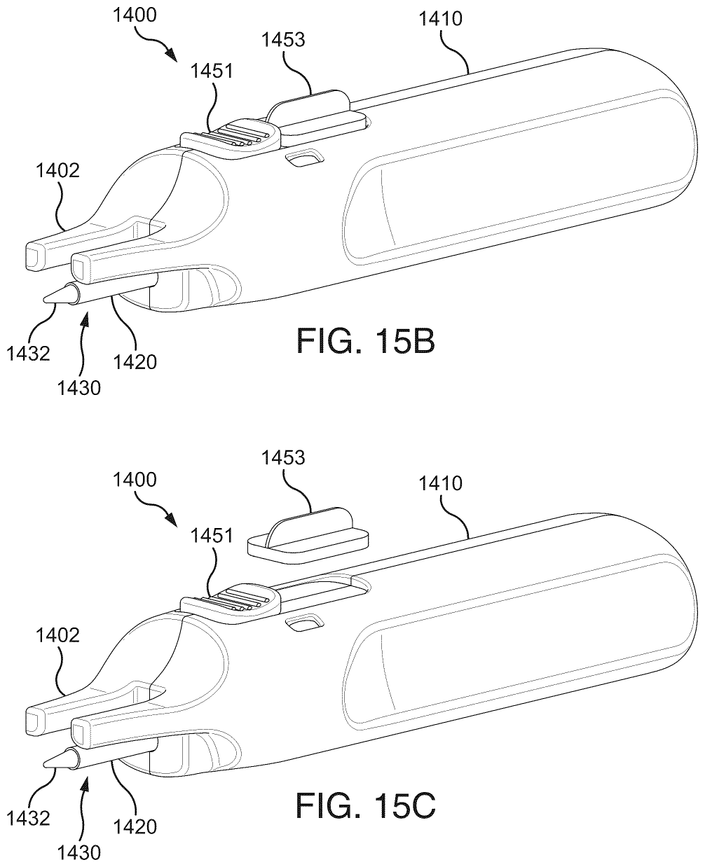

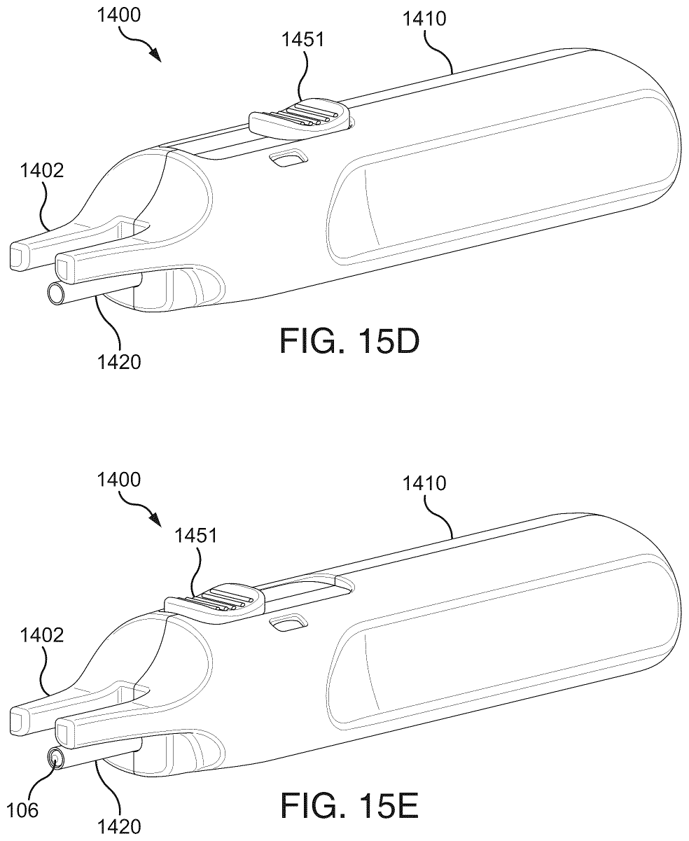

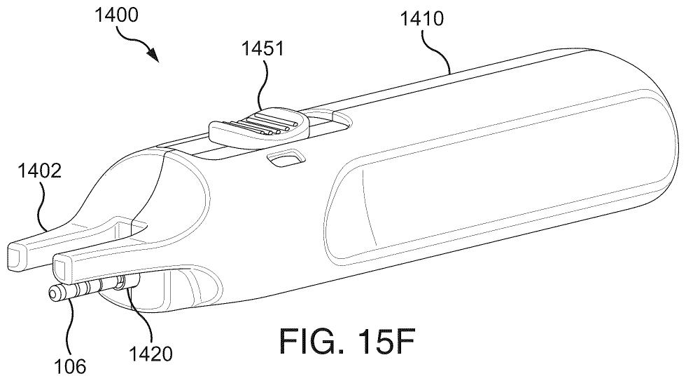

In some aspects, as shown in FIG. 15A, the insertion tool 1400 may be shipped in a package 1500. In some aspects, the package 1500 may include a base 1502 and a removable cover 1504. In some aspects, the package 1500 may include the insertion tool 1400 and a hydration fluid container 1506 (e.g., syringe) connected to the insertion tool 1400 by tubing 1508 (e.g., luer lock tubing). In some aspects, the tubing 1508 may enter the cavity 1411 in the handle 1410 via a loading port 1443 in the handle 1410. In some aspects, the hydration fluid container 1506 may introduce a hydration fluid (e.g., saline fluid) into the cannula 1442 (via the tubing 1508 and loading port 1443). In some aspects, the hydration fluid may hydrate (or at least begin hydration of) at least a portion of the device 106 (e.g., an analyte indicator or hydrogel of the device 106) before implantation of the device 106. In some aspects, the cannula 1442 may act as a hydration cavity inside the insertion tool 1400.

In some aspects, as shown in FIG. 15A, a user may open the package 1500 by removing the cover 1504. In some aspects, after the package 1500 has been opened, the user may use the hydration fluid container 1506 to provide the hydration fluid to device 106 in the cannula 1442 of the insertion tool 1400 (e.g., by activating a plunger of the hydration fluid container 1506). In some alternative aspects, when the package 1500 is opened, hydration fluid may have already been present in the tubing 1508 and/or the cannula 1442 of the insertion tool 1400 in the package 1500.

FIG. 15B shows a configuration of the insertion tool 1400 when used to create the subcutaneous pocket 102 below the skin surface 104 according to some aspects. In some aspects, the insertion tool 1400 may be used to create the subcutaneous pocket 102 below the skin surface 104 with the dissector 1430 (including a first rod 1434 and a dissector tip 1432) in an extended position in which the dissector tip 1432 protrudes out of a distal end of the tunneling tube 1420 and the stopper 1453 preventing movement of the slider knob 1451 (e.g., from a forward position), as shown in FIG. 15B. In some aspects, the subcutaneous pocket 102 may be created by inserting the tunneling tube 1420 with the dissector tip 1432 protruding from the tunneling tube 1420 through an incision in the skin surface.

In some aspects, after creating the subcutaneous pocket 102, the stopper 1453 may be removed from the insertion tool 1400 (as shown in FIG. 15C). In some aspects, after removal of the stopper 1453, the slider knob 1451 may be capable of movement (e.g., from the forward position).

In some aspects, after removing the stopper 1453, the slider knob 1451 of the actuator 1450 may be moved (e.g., from the forward position to a rear position) to cause movement of the dissector 1430 (e.g., including the tip 1432 and the first rod 1434) from its extended position to a retracted position (as shown in FIG. 15D). In some aspects, the dissector 1430 in the retracted position may be rotated from an operating position on the longitudinal axis of the tunneling tube 1420 to an idle position, and the inserter 1440 (e.g., including a cannula 1442 and a second rod 1444) may be rotated from an idle position to an operating position on the longitudinal axis of the tunneling tube 1420.

In some aspects, the slider knob 1451 of the actuator 1450 may be moved (e.g., from the rear position to the forward position) to cause movement of the inserter 1440 (e.g., including the second rod 1444 and cannula 1442) in the operating position from a retracted position toward an extended position so that the cannula 1442 (with the device 106 held therein) is disposed at least partially in the tunneling tube 1420 (as shown in FIG. 15E).

In some aspects, with the cannula 1442 disposed at least partially in the tunneling tube 1420, the slider knob 1451 of the actuator 1450 may be moved from the forward position to cause retraction of the tunneling tube 1420 toward the handle 1410 to slide the cannula 1442 along the second rod 1444 and into at least partially into the cavity 1411 of the handle 1410 (as shown in FIG. 15F). In some aspects, as the cannula 1442 retracts with the tunneling tube 1420 into the cavity 1411 of the handle 1410, the second rod 1444 may remain in the extended position (e.g., using a catch mechanism of the actuator 1450), abut against the device 106, act as a backstop, and force the device 106 at least partially out of the cannula 1442 and the tunneling tube 1420 (as shown in FIG. 15F).

In some aspects, after at least partially retracting the tunneling tube 1420 into the cavity 1411 of the handle 1410 (or simultaneously therewith), the handle 1410 may be used to pull the tunneling tube 1420 out of the subcutaneous pocket 102, and the device 106 may be left in the subcutaneous pocket 102. In some aspects, one or more of the dissector tip 1432, first rod 1434, cannula 1442, second rod 1444, actuator 1450, and rotation actuator may be moved while keeping the tunneling tube 1420 disposed in the subcutaneous pocket 102.

In some aspects, as shown in FIGS. 15B-15F, the insertion tool 1400 may include one or more guide prongs 1402 extending from a first end of the handle 1410. In some aspects, the one or more guide prongs 1402 may be configured to limit the depth at which the dissector tip 1432 is capable of creating the subcutaneous pocket 102. In some aspects, the dissector tip 1432 may extend farther from the end of the handle 110 than the one or more guide prongs 1402.

In some aspects, the blunt dissector functionality of the tool 1400 may be utilized to create the subcutaneous pocket 102 with the dissector tip 1432 and tunneling tube 1420 (e.g., a steel tunneling tube). In some aspects, the insertion tool 1400 may create the subcutaneous tunnel while the device 106 is hydrating. In some aspects, after the subcutaneous tunnel is created, the dissector 1430 may be retracted using the slider 1451 of the actuator 1450 and then a rotation actuator (e.g., a spring) may align the device 106 into the bore that is used for ejection. Then, by sliding the thumb slider forward, the device 106 may be deployed, and the tool 1400 may be pulled from the insertion site.

In some aspects, a method of creating a subcutaneous pocket 102 below a skin surface 104 and implanting a device 106 in the subcutaneous pocket 102 using the insertion tool 1400 may include a first step of using the insertion tool 1400 with the dissector tip 1432 and the first rod 1434 in the extended position to creating the subcutaneous pocket 102. In some aspects, during the first step, a tubing 1508 may seal off device 106 for hydration. In some aspects, the method may include a second step of, after removing the stopper 1453, retracting the slider knob 1451 to move the dissector 1430 to a retracted positon. In some aspects, the second step may include rotating the dissector 1430 from an operating position to an idle position and rotating the inserter 1440 from an idle positon to an operating positon. In some aspects, the method may include a third step of pushing the slider knob 1451 forward, which may push the cannula 1442 (with the device 106 held therein) at least partially in the tunneling tube 1420. In some aspects, the method may include a fourth step of retracting the slider knob 1451 to pull back the cannula 1442 and tunneling tube 1420 and release the device 106 into the subcutaneous pocket 102 created in the first step.

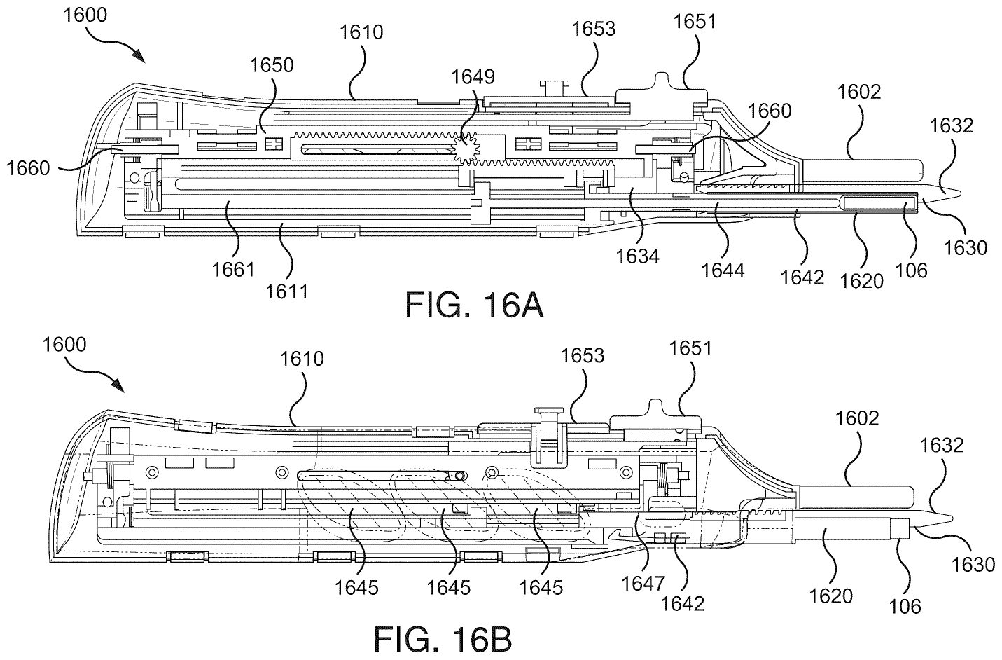

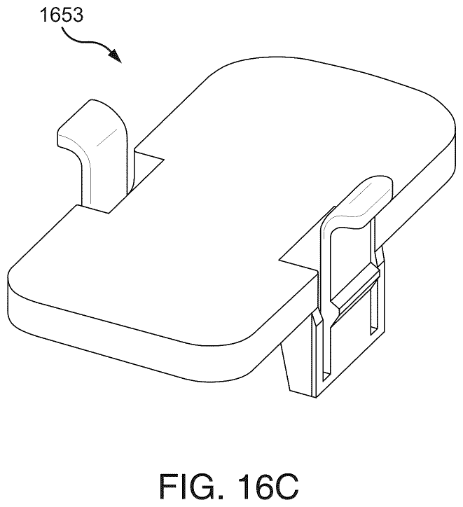

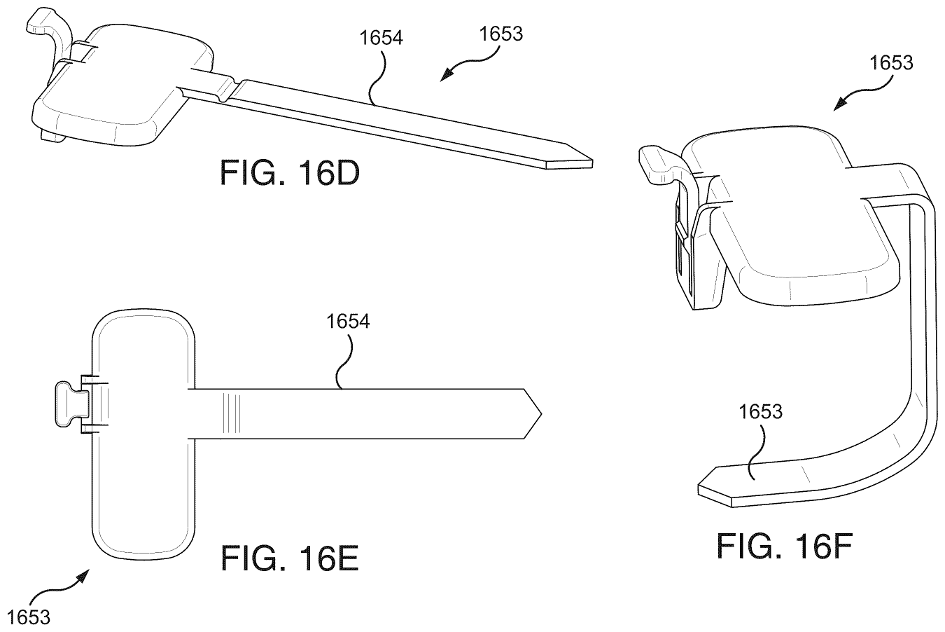

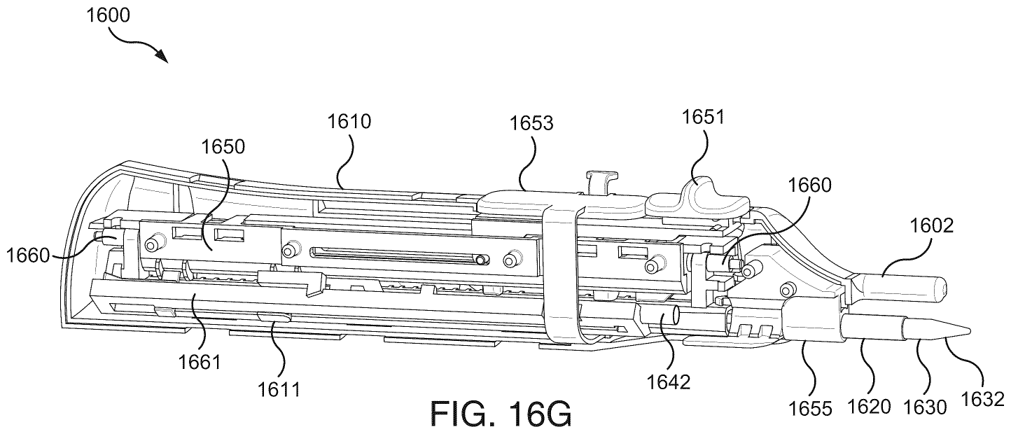

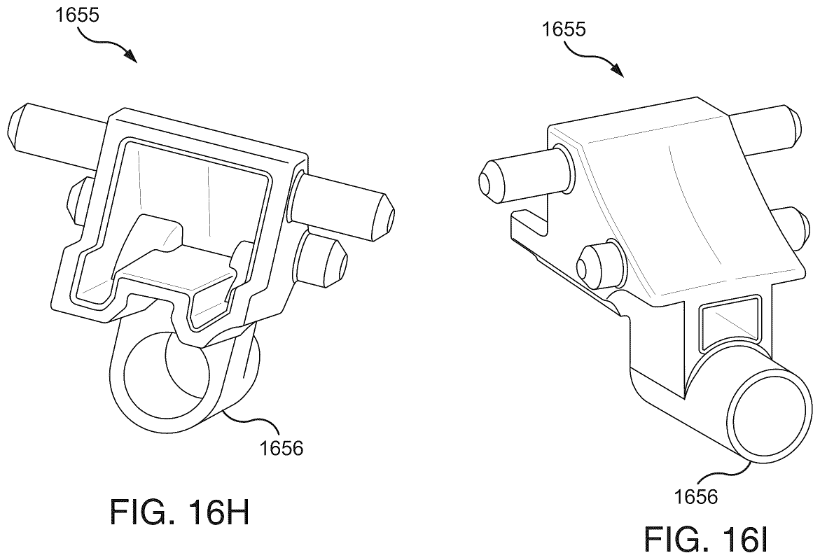

FIGS. 16A and 16B illustrate an exemplary insertion tool 1600 for creating the subcutaneous pocket 102 below the skin surface 104 and implanting the device 106 in the subcutaneous pocket 102. In some aspects, the insertion tool 1600 may include a handle 1610, a tunneling tube 1620, a dissector 1630, an inserter 1640, an actuator 1650, and a stopper 1653.

In some aspects, the insertion tool 1600 may include one or more of the same features as the aspects of the insertion tool 1400 shown in FIGS. 14A-14D. For example, the tunneling tube 1620 may be configured to move between an extended position to create the subcutaneous pocket 102 using the dissector 1630 and a retracted position to deploy the device 106 into the subcutaneous pocket 102 using the inserter 1440. In some aspects, the dissector 1630 may include a first rod 1634 configured to move along a cavity 1611 of the handle 1610 and a dissector tip 1632 coupled to a distal end of the first rod 1634. In some aspects, the inserter 1640 may include a second rod 1644 configured to move along the cavity 1611 of the handle 1610 and a cannula 1642 configured to hold the device 106 at a first end and be coupled to the second rod 1644 at a second end.

In some aspects, the dissector 1630 may be configured to move from an operating position in which a longitudinal axis of the dissector 1630 coincides with a longitudinal axis of the tunneling tube 1620 to an idle position is which the longitudinal axis of the dissector 1630 does not coincide with (e.g., is shifted away from and as is parallel to) the longitudinal axis of the tunneling tube 1620. In some aspects, the inserter 1640 may be configured to move from an idle position in which a longitudinal axis of the inserter 1640 does not coincide with (e.g., is shifted away from and as is parallel to) the longitudinal axis of the tunneling tube 1620 to an operating position in which the longitudinal axis of the inserter 1640 coincides with the longitudinal axis of the tunneling tube 1620.

In some aspects, the insertion tool 1600 may include a swing 1661. In some aspects, the swing 1661 may include one or more hinges 1660. In some aspects, the swing 1661 including the one or more hinges 1660 may be disposed in the handle 1610. In some aspects, the insertion tool 1600 may include a frame 1662, and the one or more hinges 1660 of the swing 1661 may be attached rotatably to the frame 1662. In some aspects, the swing 1661 may be configured to rotate the dissector 1630 and the inserter 1640 about the one or more hinges 1660. In some aspects, the dissector 1630 may be configured to rotate from its operating position to its idle position as the inserter 1640 rotates from its idle position to its operating position.

In some aspects, the insertion tool 1600 may include a rotation actuator operatively connected to the dissector 1630 and the inserter 1640 to cause the rotation of the dissector 1630 and the inserter 1640. In some aspects, the rotation actuator may include, for example and without limitation, a spring. In some aspects, the rotation actuator may be configured to automatically rotate the dissector 1630 from its operating position to its idle position and the inserter 1640 from its idle position to its operating position when an actuator 1650 is moved to a retracted positon. In some alternative aspects, the rotation actuator may include, for example and without limitation, a twist knob or a slider, and the rotation actuator may be configured to manually rotate the dissector 1630 and inserter 1640.

In some aspects, the actuator 1650 may be disposed in the handle 1610 and may be operatively connected to one or more of the tunneling tube 1620, the dissector 1630, and the inserter 1640. In some aspects, the actuator 1650 may be configured to selectively force one or more of the tunneling tube 1620, the dissector 1630, and the inserter 1640 to move between retracted and extended positions. In some aspects, the actuator 1650 may include a linear actuator (e.g., a rack and pinion). In some aspects, the actuator 1650 may include a track extending along the handle 1610 and a slider knob 1651 configured to slide along the track. In some aspects, the actuator 1650 may include a gear 1649 that engages with teeth. In some aspects, the gear 1649 may enable relatively small movements of the slider knob 1651 to cause relatively large movements of the dissector 1630 and/or the inserter 1640. In some aspects, the handle 1610 may have gripping elements 1645 to facilitate handling of the device.

In some aspects, the stopper 1653 of the insertion tool 1600 may be configured to prevent movement of the slider knob 1651 from a forward position and, thereby, prevent movement of the dissector tip 1632 from an extended position. In some aspects, the stopper 1653 may prevent movement (e.g., accidental movement) of the slider knob 1651 during (i) shipping/transportation of the of insertion tool 1600, (ii) sterilization of the insertion tool 1600, and/or (iii) creation of the subcutaneous pocket 102 below the skin surface 104 with the dissector tip 1632 in the extended position. In some aspects, the stopper 1653 may act as a safety feature that prevents attempted creation of the subcutaneous pocket 102 without the dissector tip 1632 at its extended position. In some aspects, the stopper 1653 may be configured to be removed from the insertion tool 1600 (e.g., after creation of the subcutaneous pocket 102 below the skin surface 104). In some aspects, after removal of the stopper 1653, the slider knob 1651 may be moved from the forward position (e.g., to move the dissector tip 1632 and first rod 1634 from their extended position to their retracted position).

In some aspects, as shown in FIGS. 16A and 16B, the stopper 1653 may include a ridge (e.g., a tab), and a user may remove the stopper 1653 from the insertion tool 1600 by pulling on the ridge. In some alternative aspects, as shown in FIGS. 16C-16F, the stopper 1653 may include one or more clips, and a user may remove the stopper 1653 from the insertion tool 1600 by squeezing the one or more clips. In some aspects, as shown in FIGS. 16D-16G, the stopper 1653 may include an extension 1654. In some aspects, as shown in FIG. 16G, the extension 1654 of the stopper 1653 may enter the cavity 1611 (e.g., through a slot or opening in the housing 1610). In some aspects, as shown in FIG. 16G, in the cavity 1611 of the housing 1610, the extension 1654 of the stopper 1653 may extend around the actuator 1650 and the swing 1661 and act as a backstop for the tunneling tube 1620 and/or dissector 1630. In this way, the stopper 1653 may be configured to both (a) prevent movement of the slider knob 1651 from a forward position and, thereby, prevent movement of the dissector tip 1632 from an extended position and/or prevent attempted creation of the subcutaneous pocket 102 without the dissector tip 1632 at its extended position and (b) prevent deflection and/or movement of the tunneling tube 1620 and/or dissector 1630 during use of the dissector 1630 to create the subcutaneous pocket 102.

In some aspects, as shown in FIG. 16B, the insertion tool 1600 may include a window 1647. In some aspects, the window 1647 may be made from a see-through material and may allow visual confirmation that the device 106 is in the insertion tool 1600. In some aspects, the device 106 may protrude slightly from the cannula 1642, and the portion of the device 106 protruding from the cannula 1642 may be visible through the window 1647.

In some aspects, as shown in FIG. 16G, the insertion tool 1600 may include a cannula support 1655. In some aspects, the cannula support 1655 may be held in place by the handle 1610. In some aspects, as shown in FIGS. 16H and 16I, the cannula support 1655 may include a passage 1656. In some aspects, as shown in FIG. 16G, when the insertion tool 1600 is assembled, the tunneling tube 1620 may pass through the passage 1656 of the cannula support 1655. In some aspects, the cannula support 1655 may be configured to minimize deflection of the tunneling tube 1620, dissector 1630, inserter 1640, and/or cannula 1642 during use of the insertion tool 1600.

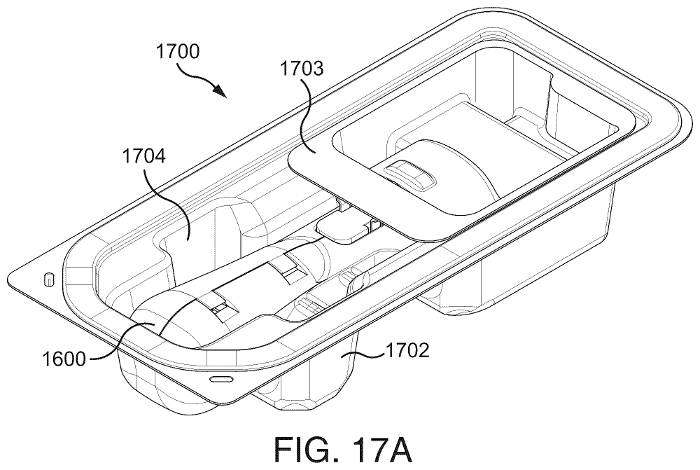

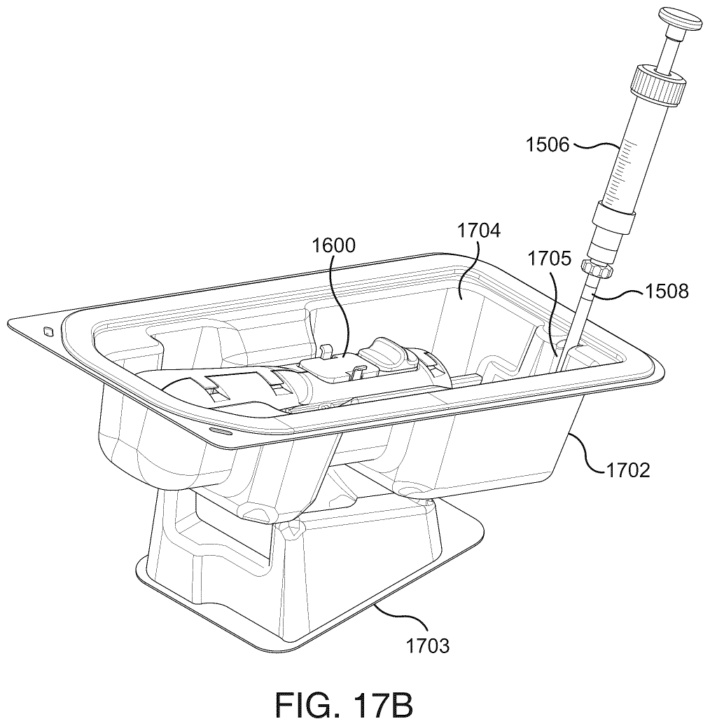

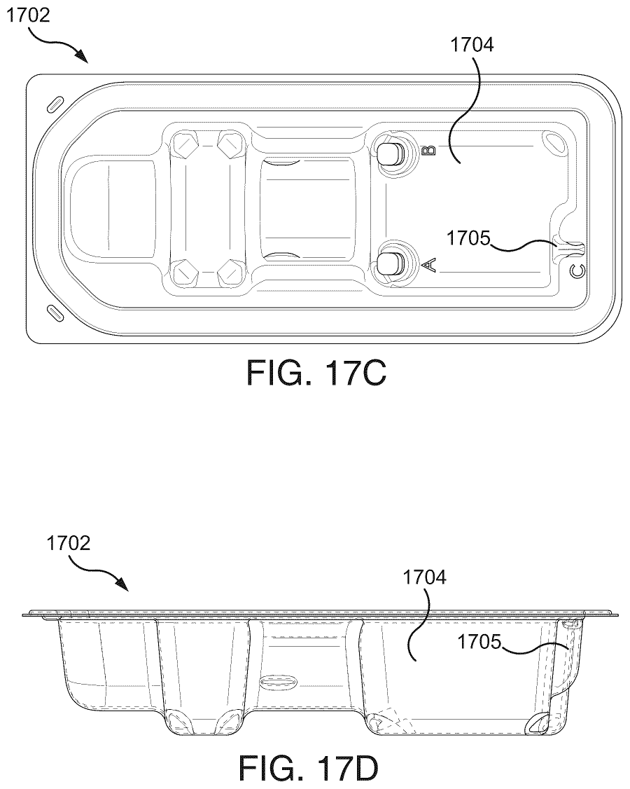

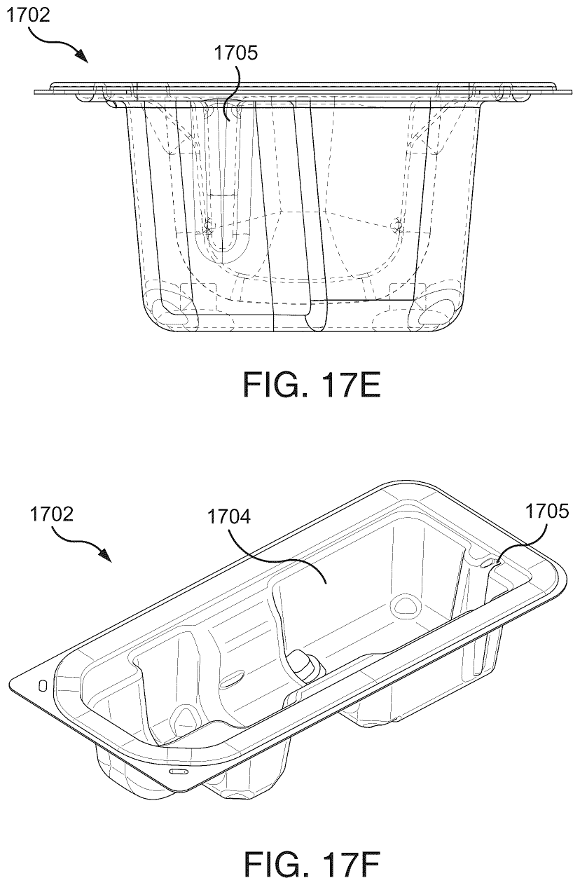

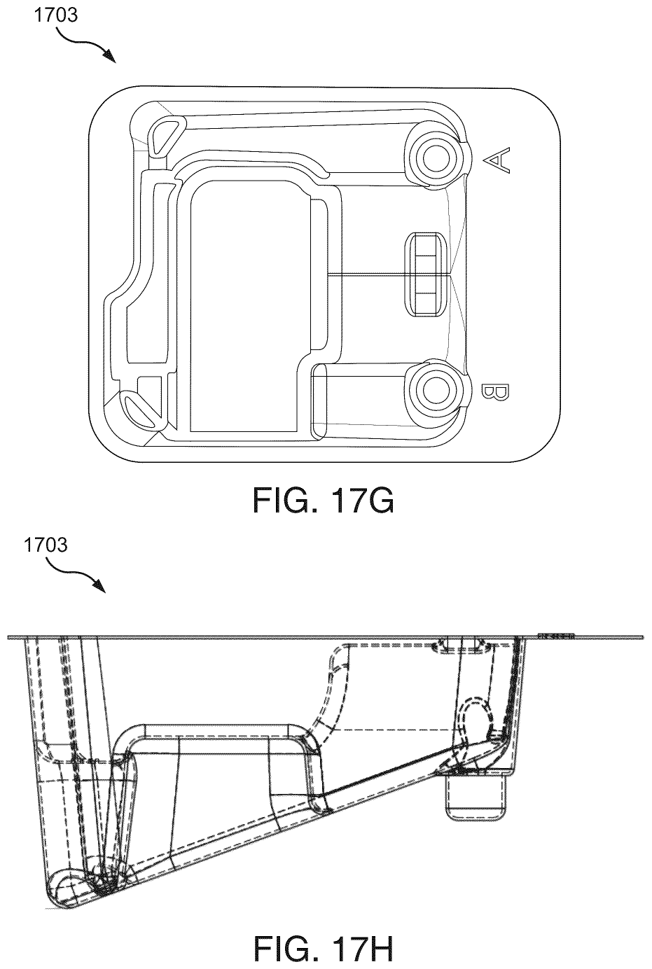

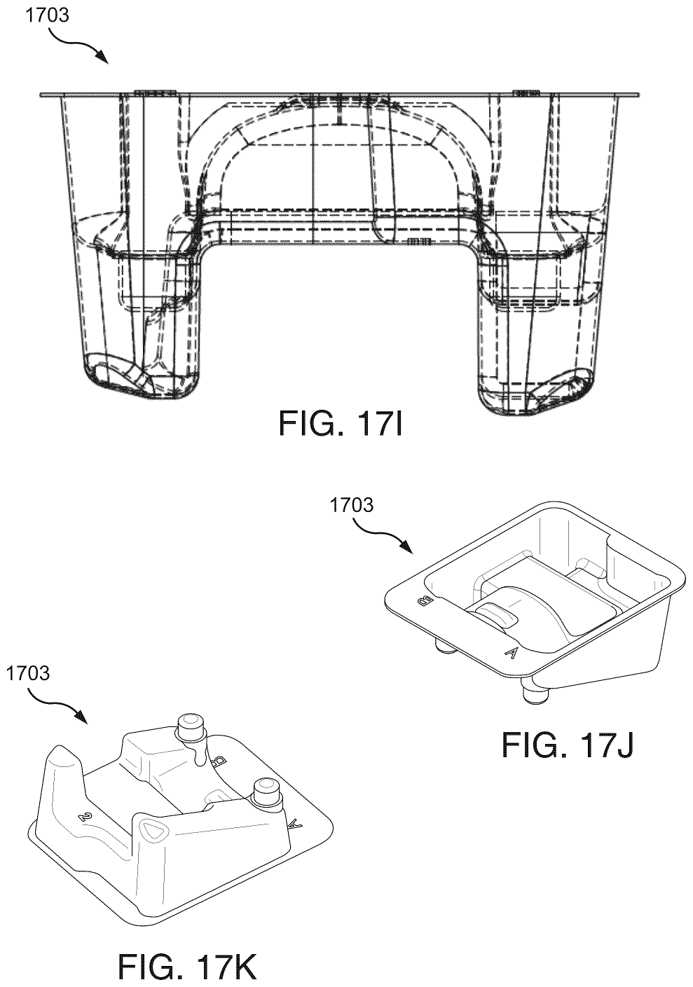

In some aspects, as shown in FIG. 17A, a product (e.g., the insertion tool 1600) may be shipped in a package 1700. In some aspects, as shown in FIGS. 17A and 17B, the package 1700 may include a base 1702. In some aspects, the base 1702 may include a cavity 1704. In some aspects, the base 1702 may be configured to support the product (e.g., the insertion tool 1600) at least partially within the cavity 1704. FIGS. 17C-17F provide additional views of the base 1702 according to some aspects.

In some aspects, as shown in FIGS. 17A and 17B, the package 1700 may include a stand 1703. In some aspects, as shown in FIG. 17A, the stand 1703 may be configured to fit at least partially within the cavity 1704 of the base 1702. In some aspects, as shown in FIG. 17A, the stand 1703 may be configured to fit fully within the cavity 1702. In some aspects, the stand 1703 may be configured to, when the stand 1703 is at least partially within the cavity 1704 of the base 1702, hold the product (e.g., the insertion tool 1600) at least partially within the cavity 1704 of the base 1702 between the stand 1703 and a bottom surface of the cavity 1704 of the base 1702. In some aspects, although not shown in FIG. 17A, the package may further include a removable cover, and the base 1702 and the cover may enclose the product and the stand 1703.

In some aspects, as shown in FIG. 17A, the base 1702 may be configured to support at least a portion of the bottom of the product (e.g., the insertion tool 1600) and at least a portion of sides of the product. In some aspects, as shown in FIG. 17A, the stand 1703 may be configured to, when the stand 1703 is at least partially within the cavity 1704 of the base 1702, hold the product at least partially within the cavity 1702 of the base by contacting at least a portion of a top of the product supported at least partially within the cavity 1704.

In some aspects, as shown in FIG. 17B, when the product is being prepared for use (e.g., for use creating a subcutaneous pocket 102 below a skin surface 104 and implanting a device 106 in the subcutaneous pocket 102), the stand 1703 may be removed from the cavity 1704 of the base 1702 and attached to an exterior bottom surface of the base 1702. In some aspects, the stand 1703 may be configured to, when the stand 1703 is attached to the exterior bottom surface of the base 1702, define an angle of tilt of the product (e.g., the insertion tool 1600) supported within the cavity of the base 1702. In some aspects, the angle of tilt of the product may be, for example, 20° with respect to a horizontal line. In some aspects in which the product is an insertion tool 1600 including a cannula 1642 and a device 106 in a passage of the cannula 1642, the angle of tilt defined by the stand 1703 may be such that hydration fluid in the cannula 1642 stays in the cannula 1642 around the device 106.

In some aspects (e.g., some aspects in which the product is an insertion tool 1600), as shown in FIG. 17B, the base 1702 may include a passage 1705 through which tubing 1508 (e.g., luer lock tubing) is capable of passing, and the tubing 1508 may be configured to connect to a hydration fluid container 1506 (e.g., syringe) to the product (e.g., the insertion tool 1600) and provide hydration fluid (e.g., saline fluid) from the hydration fluid container to the product supported within the cavity 1704 of the base 1702 to which the stand 1703 is attached. In some aspects, the tubing 1508 may enter the cavity 1611 in the handle 1610 via a loading port 1643 in the handle 1610. In some aspects, the hydration fluid container 1506 may introduce a hydration fluid into the cannula 1642 (via the tubing 1508 and loading port 1643). In some aspects, the hydration fluid may hydrate (or at least begin hydration of) at least a portion of the device 106 (e.g., an analyte indicator or hydrogel of the device 106) before implantation of the device 106. In some aspects, the cannula 1642 may act as a hydration cavity inside the insertion tool 1400. In some aspects, the angle of tilt defined by the stand 1703 attached to the base 1702 may be such that hydration fluid introduced in the cannula 1642 stays in the cannula 1642 around the device 106.

FIGS. 17G-17K provide additional views of the stand 1703 according to some aspects. As shown in FIGS. 17A, 17B, 17J, and 17K, when attached to the exterior bottom surface of the base 1702 (e.g., during hydration of the device 106), the stand 1703 may be upside down relative to its position at least partially within the cavity 1704 of the base 1702 (e.g., during shipping).

In some aspects, the package 1700 may additionally include the hydration fluid container 1506 and tubing 1508, which may be shipped with the product (e.g., insertion tool 1600). In some aspects in which the package 1700 includes a removable cover, the base 1702 and the cover may enclose the product, the stand 1703, the hydration fluid container 1506, and the tubing 1508.

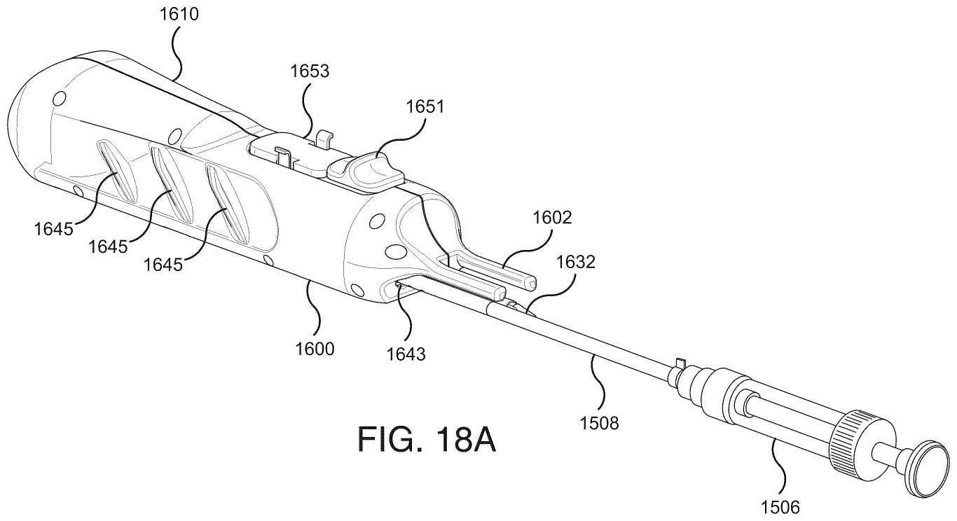

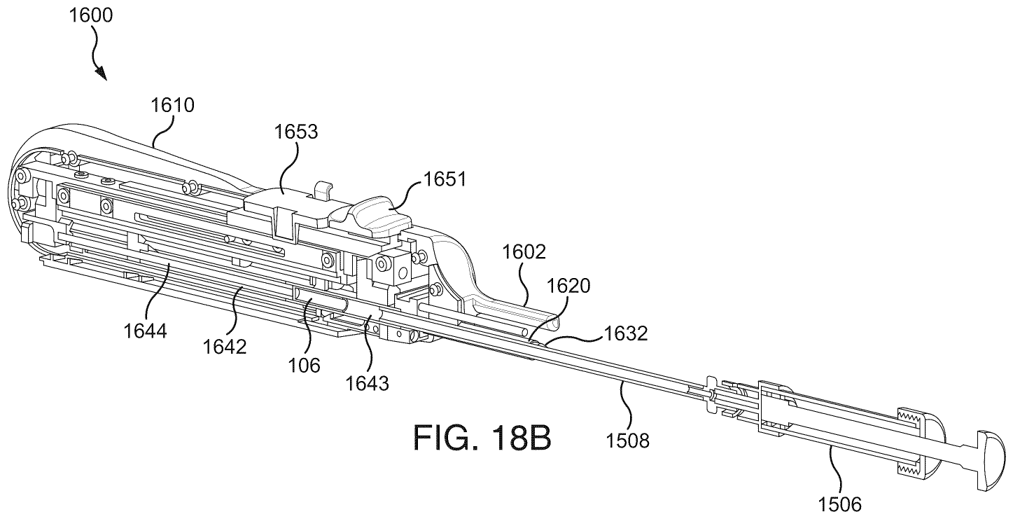

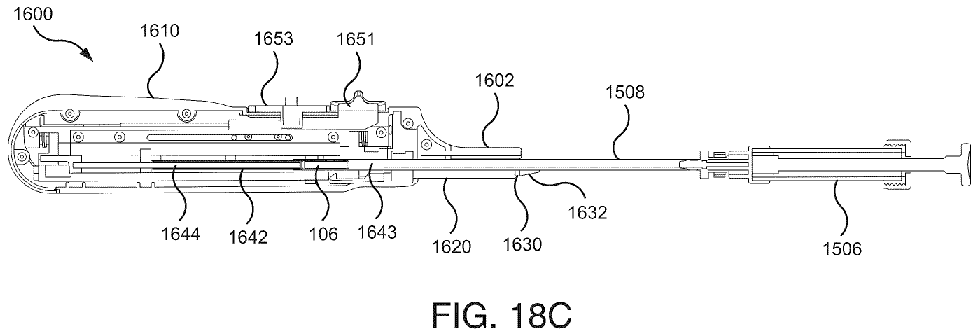

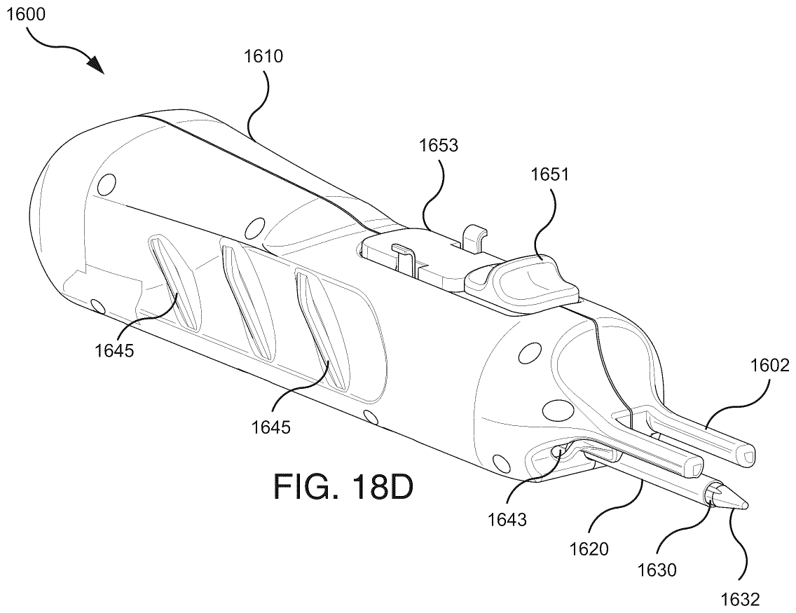

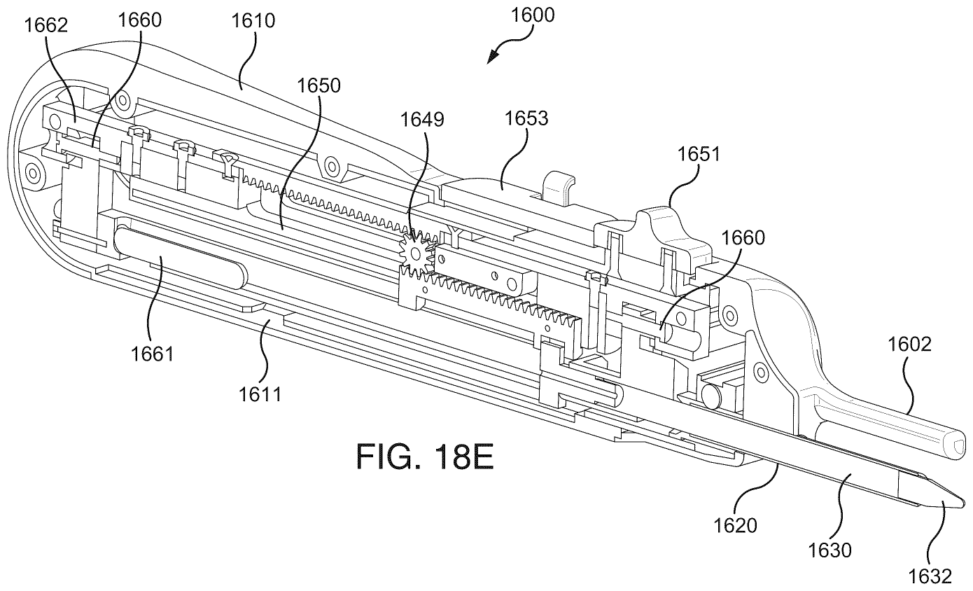

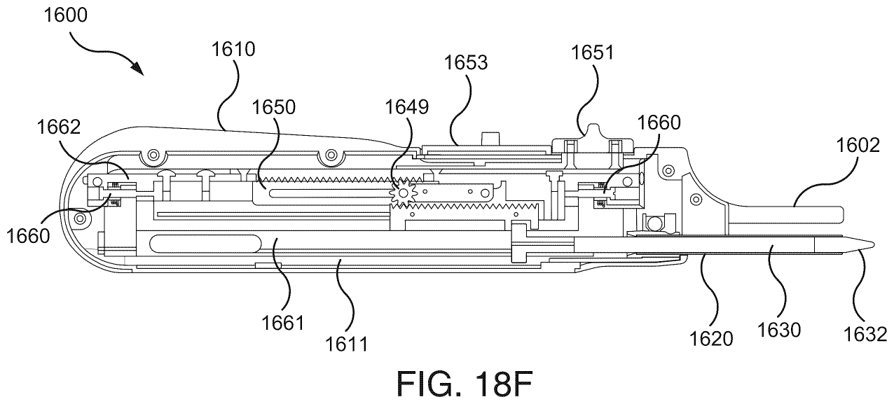

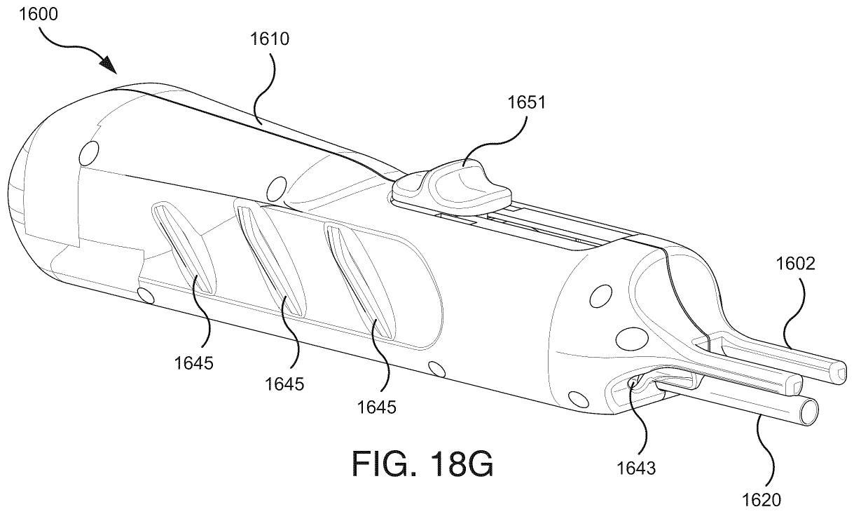

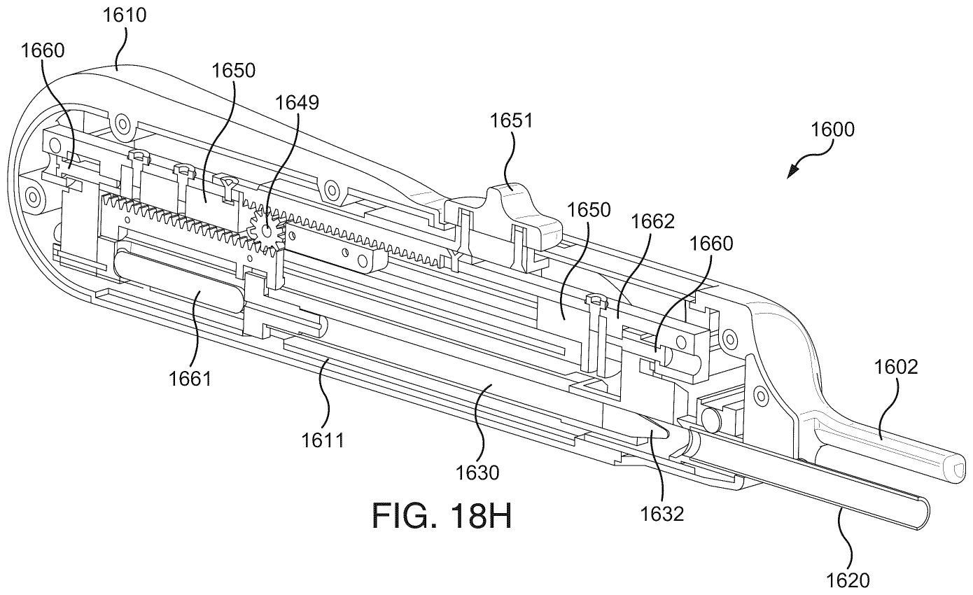

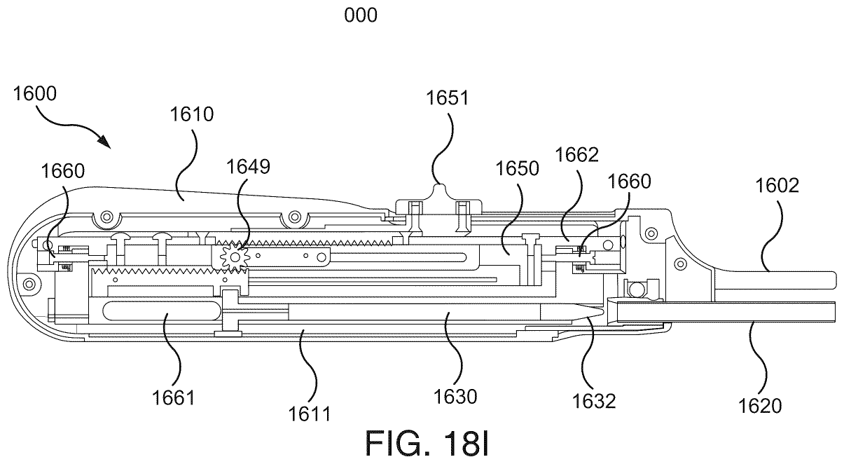

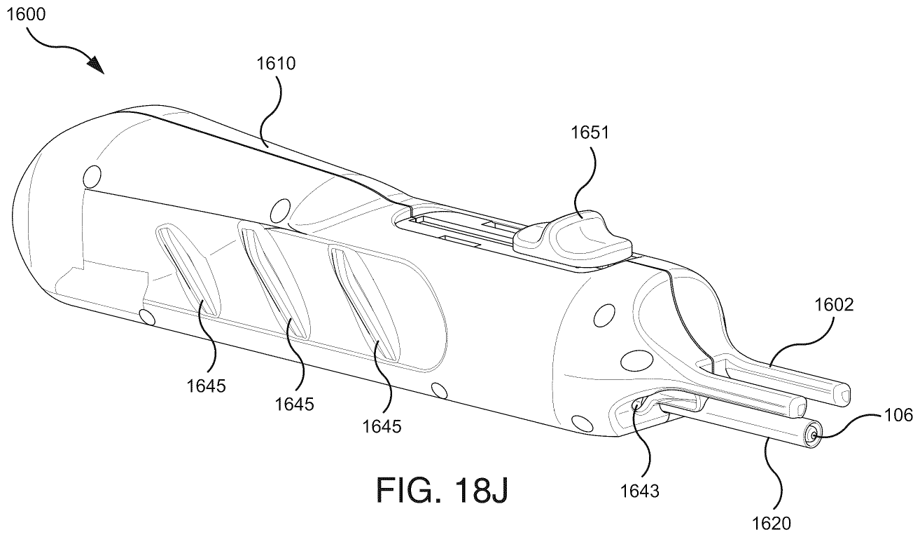

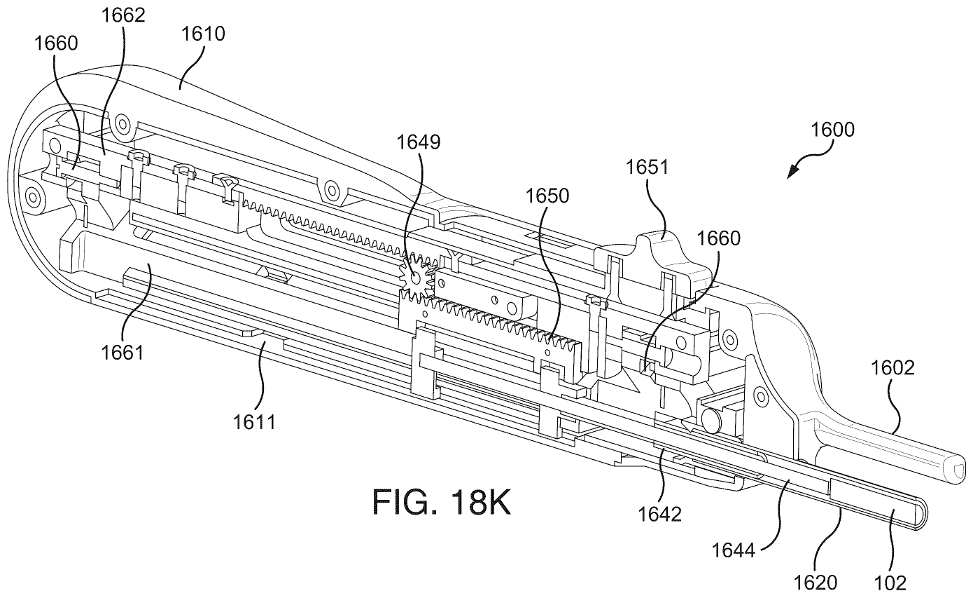

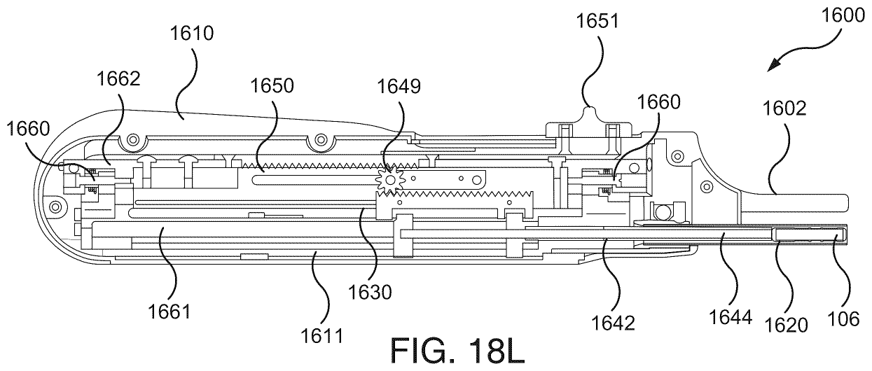

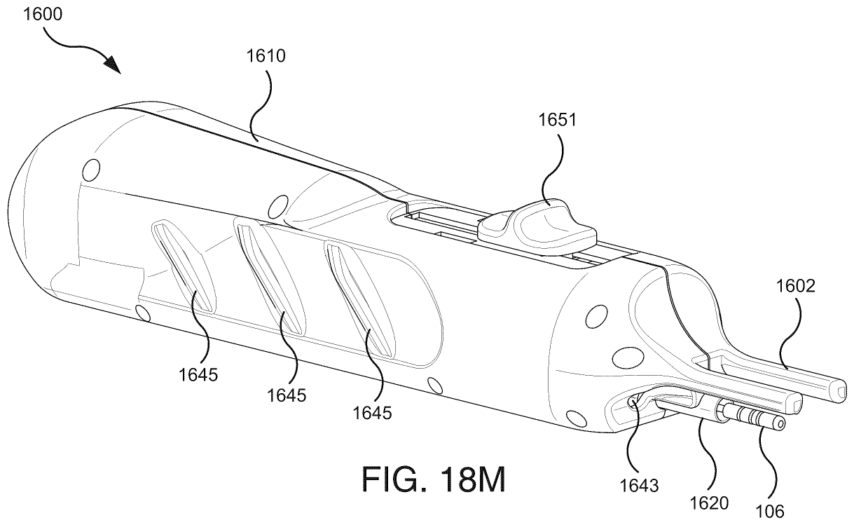

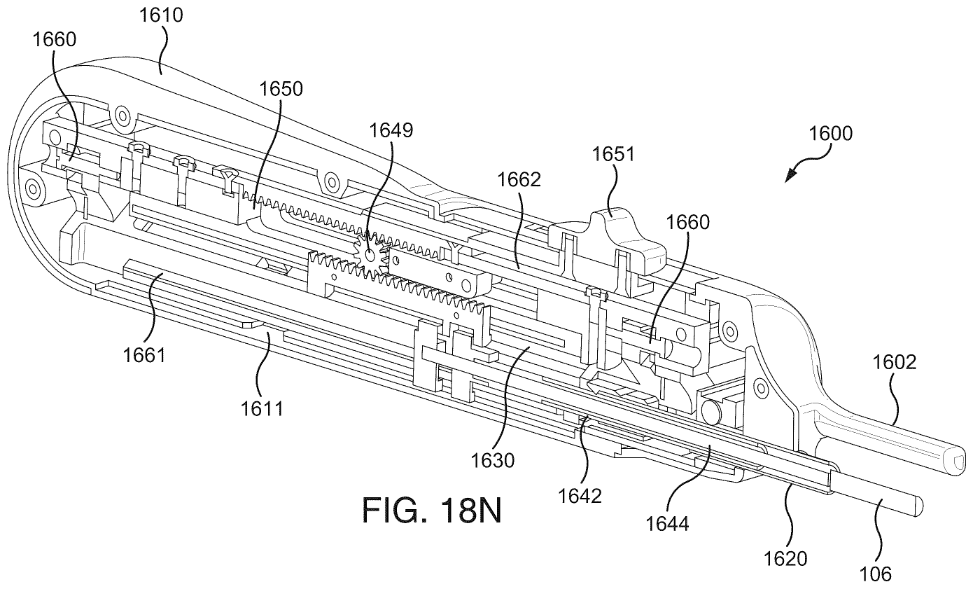

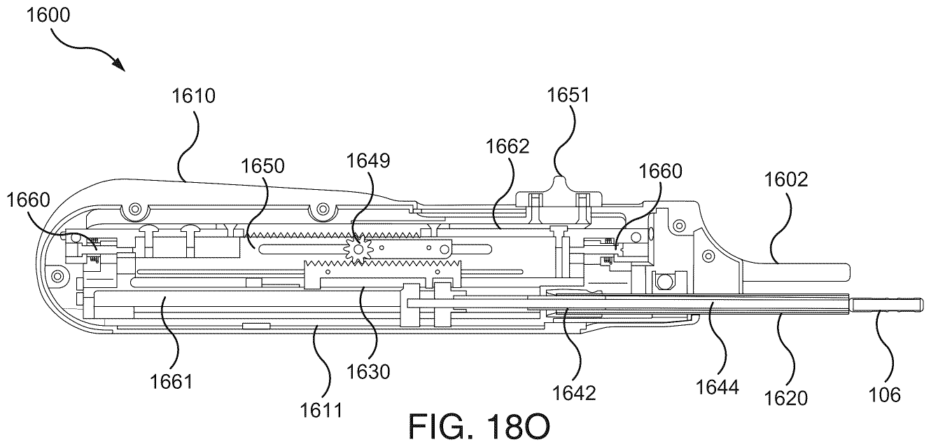

In some aspects, as shown in FIGS. 18A-18C, a user may open the package 1700 (e.g., by removing a cover that, together with the base 1702 encloses at least the insertion tool 1600 in the cavity 1704 of the base 1702). In some aspects, after the package 1700 has been opened, the user may use the hydration fluid container 1506 to provide the hydration fluid to device 106 in the cannula 1642 of the insertion tool 1600 (e.g., by activating a plunger of the hydration fluid container 1506). In some aspects, as shown in FIG. 17B, the hydration fluid may be provided to insertion tool 1600 with (i) the insertion tool 1600 supported by the base 1702 and at least partially within the cavity 1704 of the base 1702 and (ii) the stand 1703 attached to the exterior bottom surface of the base 1702 and defining an angle of tilt of the insertion tool 1600. In some aspects, the angle of tilt defined by the stand 1703 may be such that hydration fluid in the cannula 1642 stays in the cannula 1642 around the device 106. In some aspects, the hydration fluid may be provided to insertion tool 1600 via tubing 1508 passing through a passage 1705 of the base 1702. In some alternative aspects, when the package 1700 is opened, hydration fluid may have already been present in the tubing 1508 and/or the cannula 1642 of the insertion tool 1600 in the cavity 1704 of the package 1600.

FIGS. 18D-18F illustrate a configuration of the insertion tool for creating the subcutaneous pocket 102 below the skin surface 104 according to some aspects. In some aspects, the insertion tool 1600 may create the subcutaneous pocket 102 below the skin surface 104 with the dissector 1630 (e.g., including the dissector tip 1632 and the first rod 1634) in an extended position and the stopper 1653 preventing movement of the slider knob 1651 (e.g., from a forward position), as shown in FIGS. 18D-18F). In some aspects, the subcutaneous pocket 102 may be created by inserting the tunneling tube 1620 with the dissector tip 1632 protruding from the tunneling tube 1620 through an incision in the skin surface 104.

In some aspects, after creating the subcutaneous pocket 102, the stopper 1653 may be removed from the insertion tool 1600 (as shown in FIGS. 18G-18I). In some aspects, after removal of the stopper 1653, the slider knob 1651 may be capable of movement (e.g., from the forward position).

In some aspects, after removing the stopper 1653, the slider knob 1651 of the actuator 1650 may be moved (e.g., from the forward position to a rear position) to cause movement of the dissector 1630 (e.g., including the dissector tip 1632 and the first rod 1634) from the extended position to a retracted position (as shown in FIGS. 18G-18I). In some aspects, the dissector 1630 in the retracted position may be rotated from an operating position on the longitudinal axis of the tunneling tube 1620 to the idle position, and the inserter 1640 (e.g., including the second rod 1644 and the cannula 1642) may be rotated from the idle position to the operating position on the longitudinal axis of the tunneling tube 1620.

In some aspects, the slider knob 1651 of the actuator 1650 may be moved (e.g., from the rear position to the forward position) to cause movement of inserter 1640 (e.g., including the second rod 1644 and cannula 1642) in the operating position from a retracted position toward an extended position so that the cannula 1642 (with the device 106 held therein) is disposed at least partially in the tunneling tube 1620 (as shown in FIGS. 18J-18L).

In some aspects, with the cannula 1642 disposed at least partially in the tunneling tube 1620, the slider knob 1651 of the actuator 1650 may be moved (e.g., from the forward position) to cause retraction of the tunneling tube 1620 toward the handle 1610 to slide the cannula 1642 along the second rod 1644 and into at least partially into the cavity 1611 of the handle 1610 (as shown in FIGS. 18M-180). In some aspects, as the cannula 1642 retracts with the tunneling tube 1620 into the cavity 1611 of the handle 1610, the second rod 1644 may remain in the extended position (e.g., using a catch mechanism of the actuator 1650), abut against the device 106, act as a backstop, and force the device 106 at least partially out of the cannula 1642 and the tunneling tube 1620 (as shown in FIGS. 18M-180).

In some aspects, after at least partially retracting the tunneling tube 1620 into the cavity 1611 of the handle 1610 (or simultaneously therewith), the handle 1610 may be used to pull the tunneling tube 1620 out of the subcutaneous pocket 102, and the device 106 may be left in the subcutaneous pocket 102. In some aspects, one or more of the dissector tip 1632, first rod 1634, cannula 1642, second rod 1644, actuator 1650, and rotation actuator may be moved while keeping the tunneling tube 1620 disposed in the subcutaneous pocket 102.

In some aspects, as shown in FIGS. 18A-180, the insertion tool 1600 may include one or more guide prongs 1602 extending from a first end of the handle 1610. In some aspects, the one or more guide prongs 1602 may be configured to limit the depth at which the dissector tip 1632 is capable of creating the subcutaneous pocket 102. In some aspects, the dissector tip 1632 may extend farther from the end of the handle 110 than the one or more guide prongs 1602.

In some aspects, the blunt dissector functionality of the tool 1600 may be utilized to create the subcutaneous pocket 102 with the dissector tip 1632 and tunneling tube 1420 (e.g., a steel tunneling tube). In some aspects, the insertion tool 1600 may create the subcutaneous tunnel while the device 106 is hydrating. In some aspects, after the subcutaneous tunnel is created, the dissector 1630 may be retracted using the slider 1651 of the actuator 1650 and then a rotation actuator (e.g., a spring) may align the device 106 into the bore that is used for ejection. Then, by sliding the thumb slider forward, the device 106 may be deployed, and the insertion tool 1600 may be pulled from the insertion site.

In some aspects, a method of creating a subcutaneous pocket 102 below a skin surface 104 and implanting a device 106 in the subcutaneous pocket 102 using an insertion tool 1600 may include a step of using the insertion tool 1600 with the dissector tip 1632 and the first rod 1634 in the extended position to creating the subcutaneous pocket 102. In some aspects, during the first step, a tubing 1508 may seal off device 106 for hydration. In some aspects, the method may include a second step of, after removing the stopper 1653, retracting the slider knob 1651 to move the dissector 1630 to a retracted positon. In some aspects, the second step may include rotating the dissector 1630 from an operating position to an idle position and rotating the inserter 1640 from an idle positon to an operating positon. In some aspects, the method may include a third step of pushing the slider knob 1651 forward, which may push the cannula 1642 (with the device 106 held therein) at least partially in the tunneling tube 1620. In some aspects, the method may include a fourth step of retracting the slider knob 1651 to pull back the cannula 1642 and tunneling tube 1620 and release the device 106 into the subcutaneous pocket 102 created in the first step.

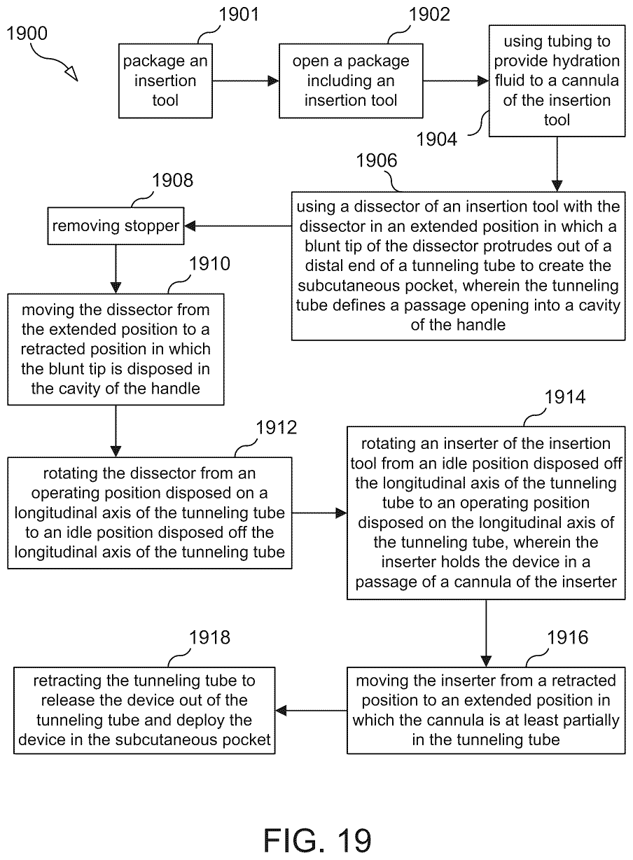

FIG. 19 is a flow chart showing a process 1900 of creating a subcutaneous pocket 102 below a skin surface 104 and implanting a device 106 in the subcutaneous pocket 102 embodying aspects of the present disclosure. In some aspects, the insertion tool 1400 described above with reference to FIGS. 14A-15F or the insertion tool 1600 described above with reference to FIGS. 16A-180 may be used to create the subcutaneous pocket 102 and implant the device 106 according to the process 1900 described in FIG. 19.

In some aspects, as shown in FIG. 19, the process 1900 may include a step 1901 of packaging an insertion tool 1400 or 1600 in a package 1500 or 1700. In some aspects, the package 1500 or 1700 may enclose the insertion tool 1400 or 1600, the hydration fluid container 1506, and/or the tubing 1508 connecting the hydration fluid container 1506 and the insertion tool 1400 or 1600 (e.g., in a cavity of a base 1502 or 1702 of the package 1500 or 1700). In some aspects using the package 1700, the step 1901 may include using the base 1702 to support the insertion tool 1400 or 1600 at least partially within the cavity 1704 of the base 1704. See FIG. 17A. In some aspects, the step 1901 may include fitting a stand 1703 at least partially within the cavity 1704 of the base 1702. In some aspects, as shown in FIG. 17A, the stand 1703 at least partially within the cavity 1704 of the base 1702 may hold the insertion tool 1600 at least partially within the cavity 1704 of the base 1702 between the stand 1703 and a bottom surface of the cavity 1704 of the base 1702. In some aspects, the step 1901 may include attaching a removable cover (e.g., cover 1504) to the base 1502 or 1702.

In some aspects, as shown in FIG. 19, the process 1900 may include a step 1902 of opening a package 1500 or 1700. In some aspects, as shown in FIG. 15A, opening the package 1500 or 1700 may include removing a cover (e.g., cover 1504) of the package 1500 or 1700 from a base 1502 or 1702 of the package 1500 or 1700.

In some aspects, as shown in FIG. 19, the process 1900 may include a step 1904 of using the tubing 1508, which may be connected to a cannula 1442 or 1642 of the insertion tool 1400 or 1600 (e.g., via the loading port 1443 or 1643 of the insertion tool 1400 or 1600), to provide hydration fluid to the passage of the cannula 1442 or 1642. In some aspects, the tubing 1508 may connect the hydration fluid container 1506 and the cannula 1442 or 1642, and the step 1904 may include using the hydration fluid container 1506 to provide the hydration fluid to the passage of the cannula 1442 or 1642 via the tubing 1508. See FIGS. 15A, 17B, and 18A-18C. In some aspects, the tubing 1508 may be disconnected from the cannula 1442 (e.g., after using the dissector 1430 to create the subcutaneous pocket 102).

In some aspects using the package 1700, the step 1902 may include removing the stand 1703 from the cavity 1704 of the base 1702. In some aspects, the step 1902 may include attaching the stand 1703 to an exterior bottom surface of the base 1702, and the stand 1703 attached to the exterior bottom surface of the base 1702 may define an angle of tilt of the insertion tool 1400 or 1600 supported within the cavity 1704 of the base 1702. See FIG. 17B. In some aspects, using the tubing 1508 to connect the hydration fluid container 1506 to the insertion tool 1400 or 1600 may include passing the tubing 1508 through the passage 1705 of the base 1702. See FIG. 17B.

In some aspects, as shown in FIG. 19, the process 1900 may include a step 1906 of using the dissector 1430 or 1630 of the insertion tool 1400 or 1600 with the dissector 1430 or 1630 in an extended position in which a blunt tip 1432 or 1632 of the dissector 1430 or 1630 protrudes out of a distal end of a tunneling tube 1420 or 1620 to create the subcutaneous pocket 102. See FIGS. 15B and 18D-18F. In some aspects, the tunneling tube 1420 or 1620 may define a passage opening into a cavity 1411 or 1611 of the handle 1410 or 1610. In some aspects, the step 1606 may include using the stopper 1453 or 1653 of the insertion tool 1400 or 1600 to prevent movement of the slider knob 1451 or 1651 while the dissector 1430 or 1630 is used create the subcutaneous pocket 102.

In some aspects, as shown in FIG. 19, the process 1900 may include a step 1908 of removing the stopper 1453 or 1653. See FIG. 15C. In some aspects, removal of the stopper 1453 or 1653 may allow movement of the slider knob 1451 or 1651 and, therefore, movement of the dissector 1430 or 1630 from the extended position.

In some aspects, as shown in FIG. 19, the process 1900 may include a step 1910 of moving the dissector 1430 or 1630 from the extended position to a retracted position in which the blunt tip 1432 or 1632 is disposed in the cavity of the handle 1410 or 1610. See FIGS. 15D and 18G-18I. In some aspects, the step 1910 may include moving a slider knob 1451 or 1651 of an actuator 1450 or 1650 of the insertion tool 1400 or 1600 in a first direction (e.g., toward the rear of the handle 1410 or 1610) to move the dissector 1430 or 1630 from the extended position to the retracted position in which the blunt tip 1432 or 1632 is disposed in the cavity of the handle 1410 or 1610. In some aspects, moving the slider knob 1451 or 1651 in the first direction to move the dissector 1430 or 1630 from the extended position to the retracted position in which the blunt tip 1432 or 1632 is disposed in the cavity of the handle 1410 or 1610 may occur after (and be enabled by) removal of the stopper 1453 or 1653. In some aspects, the dissector 1430 or 1630 may include a first rod 1434 or 1634 configured to move along the cavity 1411 or 1611 of the handle 1410 or 1610 and the passage of the tunneling tube 1420 or 1620, and the blunt tip 1432 or 1632 may be coupled to the first rod 1434 or 1634 such that the first rod 1434 or 1634 is configured to move the blunt tip 1432 or 1632 along the cavity 1411 or 1611 of the handle 1410 or 1610 and the passage of the tunneling tube 1420 or 1620 between the retracted and extended positions.

In some aspects, as shown in FIG. 19, the process 1900 may include a step 1912 of rotating the dissector 1430 or 1630 from an operating position disposed on a longitudinal axis of the tunneling tube 1420 or 1620 to an idle position disposed off the longitudinal axis of the tunneling tube 1420 or 1620.

In some aspects, as shown in FIG. 19, the process 1900 may include a step 1914 of rotating an inserter 1440 or 1640 of the insertion tool 1400 or 1600 from an idle position disposed off the longitudinal axis of the tunneling tube 1420 or 1620 to an operating position disposed on the longitudinal axis of the tunneling tube 1420 or 1620. In some aspects, the inserter 1400 or 1600 may hold the device 106 in a passage of the cannula 1442 or 1642 of the inserter 1440 or 1640. In some aspects, rotating the dissector 1430 or 1630 in step 1912 and rotating the inserter 1440 or 1640 may occur simultaneously. In some aspects, rotating the dissector 1430 or 1630 in step 1912 and/or rotating the inserter may include using a swing 1461 or 1661 rotatably coupled to the dissector 1430 or 1630 and the inserter 1440 or 1640.

In some aspects, as shown in FIG. 19, the process 1900 may include a step 1916 of moving the inserter 1440 or 1640 from a retracted position to an extended position in which the cannula 1442 or 1642 is at least partially in the tunneling tube 1442 or 1642. See FIGS. 15E and 18J-18L. In some aspects, the step 1916 may include moving the slider knob 1451 or 1651 in a second direction (e.g., toward the tunneling tube 1420 or 1620) to move the inserter 1440 or 1640 from the retracted position to the extended position. In some aspects, moving the inserter 1440 or 1640 from the retracted position to the extended position may occur after rotation of the inserter 1440 or 1640 in the step 1914 and while the inserter 1440 or 1640 is in the operating position disposed on the longitudinal axis of the tunneling tube 1420 or 1620. In some aspects, the insertor 1440 or 1640 may include a second rod 1444 or 1644 configured to move along the cavity of the handle 1410 or 1610 and the passage of the tunneling tube 1420 or 1620. In some aspects, the cannula 1442 or 1642 may include a first end configured to hold and release the device 106 and a second end coupled to the second rod 1444 or 1644 such that the second rod 1444 or 1644 is configured to move the cannula 1442 or 1642 along the cavity 1411 or 1611 of the handle 1410 or 1610 and the passage of the tunneling tube 1420 or 1620 between the retracted and extended positions.

In some aspects, as shown in FIG. 19, the process 1900 may include a step 1918 of retracting the tunneling tube 1420 or 1620 to release the device 106 out of the tunneling tube 1420 or 1620 and deploy the device 106 in the subcutaneous pocket 102. See FIGS. 15F and 18M-180. In some aspects, the step 1918 may include moving the slider knob 1451 or 1651 in the first direction to retract the tunneling tube 1420 or 1620. In some aspects, the cannula 1442 or 1642 may retract with tunneling tube 1420 or 1620, and the step 1918 may include using the second rod 1440 or 1640 to force the device 106 at least partially out of the cannula 1442 or 1642 and the tunneling tube 1420 or 1620.

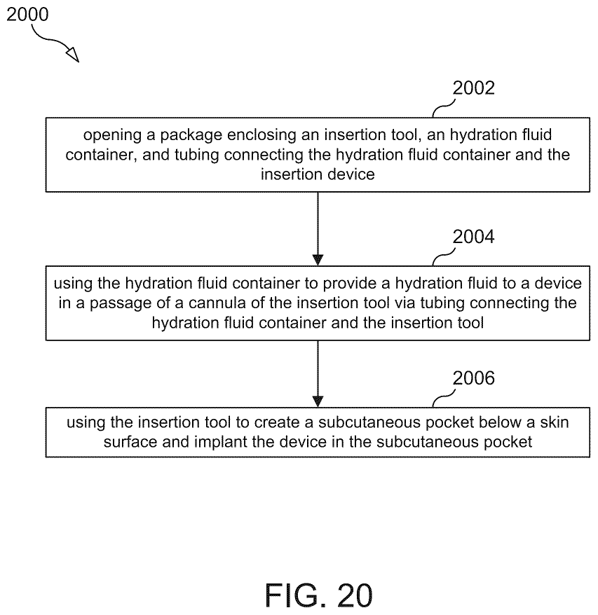

FIG. 20 is a flow chart showing a process 2000 of creating a subcutaneous pocket 102 below a skin surface 104 and implanting a device 106 in the subcutaneous pocket 102 embodying aspects of the present disclosure. In some aspects, the insertion tool 1400 described above with reference to FIGS. 14A-15F or the insertion tool 1600 described above with reference to FIGS. 16A-180 may be used to create the subcutaneous pocket 102 and implant the device 106 according to the process 2000 described in FIG. 20.

In some aspects, as shown in FIG. 20, the process 2000 may include a step 2002 of opening a package 1500 or 1700 enclosing an insertion tool 1400 or 1600, a hydration fluid container 1506, and tubing 1508 connecting the hydration fluid container 1506 and the insertion tool 1400 or 1600. In some aspects, opening the package 1500 or 1700 may include removing a cover (e.g., cover 1504) of the package 1500 or 1700 from a base 1502 or 1702 of the package 1500 or 1700. In some aspects, the insertion tool 1400 or 1600 may include a tunneling tube 1420 or 1620 and a dissector tip 1432 or 1632 protruding from the tunneling tube 1420 or 1620.

In some aspects, as shown in FIG. 20, the process 2000 may include a step 2004 of using the hydration fluid container 1506 to provide a hydration fluid to a device 106 in a passage of a cannula 1442 or 1642 of the insertion tool 1400 or 1600 via the tubing 1508 connecting the hydration fluid container 1506 and the insertion tool 1400 or 1600. In some aspects, using the hydration fluid container 1506 to provide the hydration fluid in the step 1506 may include activating a plunger of the hydration fluid container 1506. In some aspects, the hydration fluid container 1506 may provide the hydration fluid to the device 106 in the passage of the cannula 1442 or 1642 via the tubing 1508 and a loading port 1443 or 1643 of the insertion tool 1400 or 1600.

In some aspects, as shown in FIG. 20, the process 2000 may include a step 2006 of using the insertion tool 1400 or 1600 to create a subcutaneous pocket 102 below a skin surface 104 and implant the device 106 in the subcutaneous pocket 102. In some aspects, the step 2006 may include one or more of the steps of the process 1900 illustrated in FIG. 19 (e.g., one or more of steps 1906-1918).

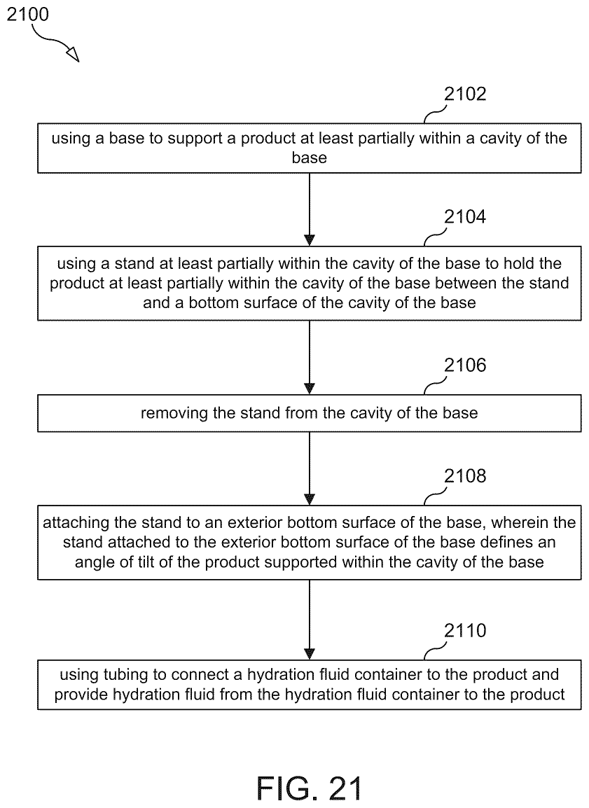

FIG. 21 is a flow chart showing a process 2100 embodying aspects of the present disclosure. In some aspects, as shown in FIG. 21, the process 2100 may include a step 2102 of using a base 1702 to support a product (e.g., insertion tool 1400 or 1600) at least partially within a cavity 1704 of the base 1703. See FIGS. 17A and 17B. In some aspects, as shown in FIG. 21, the process 2100 may include a step 2104 of using a stand 1703 at least partially within the cavity 1704 of the base 102 to hold the product (e.g., insertion tool 1400 or 1600) at least partially within the cavity 1704 of the base 1702 between the stand 1703 and a bottom surface of the cavity 104 of the base 104. See FIG. 17A. In some aspects, as shown in FIG. 21, the process 2100 may include a step 2106 of removing the stand 1703 from the cavity 104 of the base 1702. In some aspects, as shown in FIG. 21, the process 2100 may include a step 2108 of attaching the stand 1703 to an exterior bottom surface of the base 1702, and the stand 1703 attached to the exterior bottom surface of the base 1702 may define an angle of tilt of the product (e.g., insertion tool 1400 or 1600) supported within the cavity 1703 of the base 1702. See FIG. 17B.

In some aspects, as shown in FIG. 21, the process 2100 may include a step 2110 of using tubing 1508 to connect a hydration fluid container 1506 to the product (e.g., insertion tool 1400 or 1600) and provide hydration fluid from the hydration fluid container 1506 to the product. In some aspects, using the tubing 1508 to connect the hydration fluid container to the product in step 2110 may include passing the tubing 1506 through a passage 1705 of the base 1702. In some aspects, the process 2100 may include an initial step of removing a cover from the base 1702, and the base 1702 and the cover may enclose the product and the stand 1703 before the cover is removed.

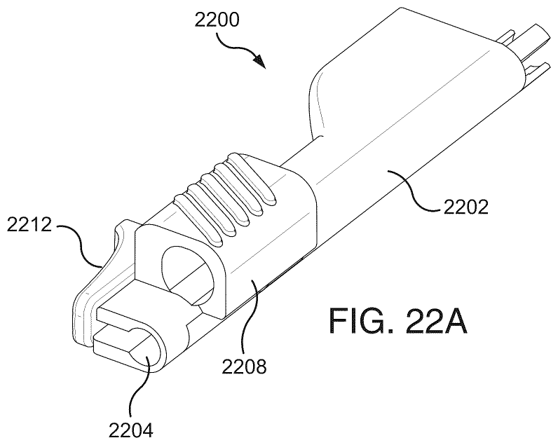

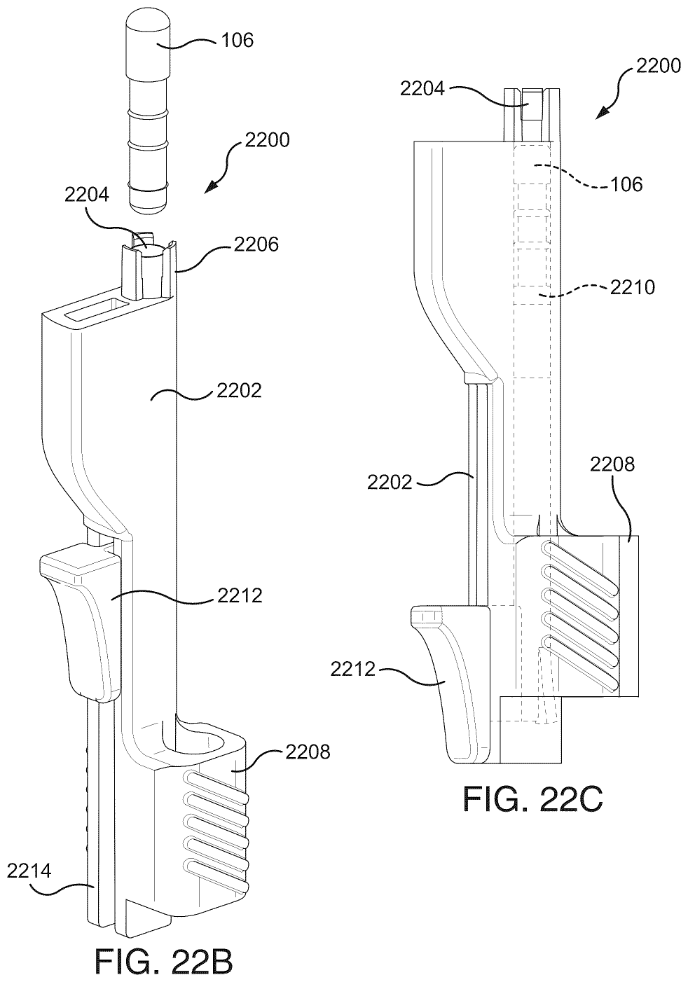

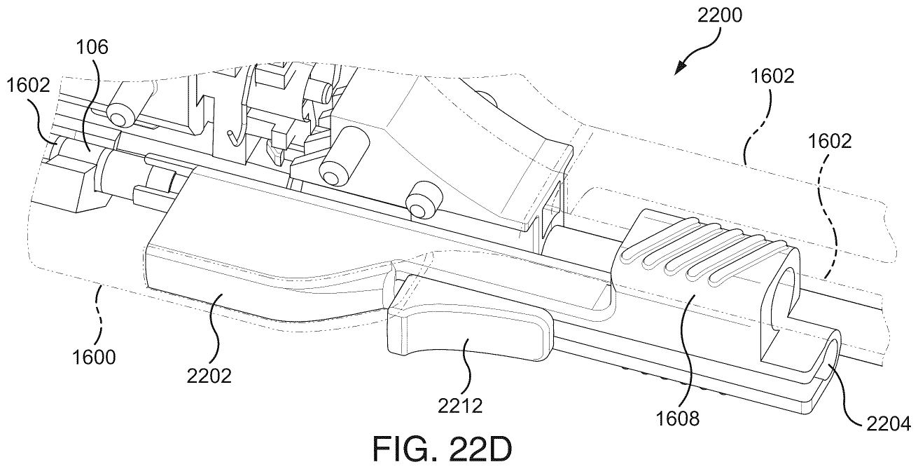

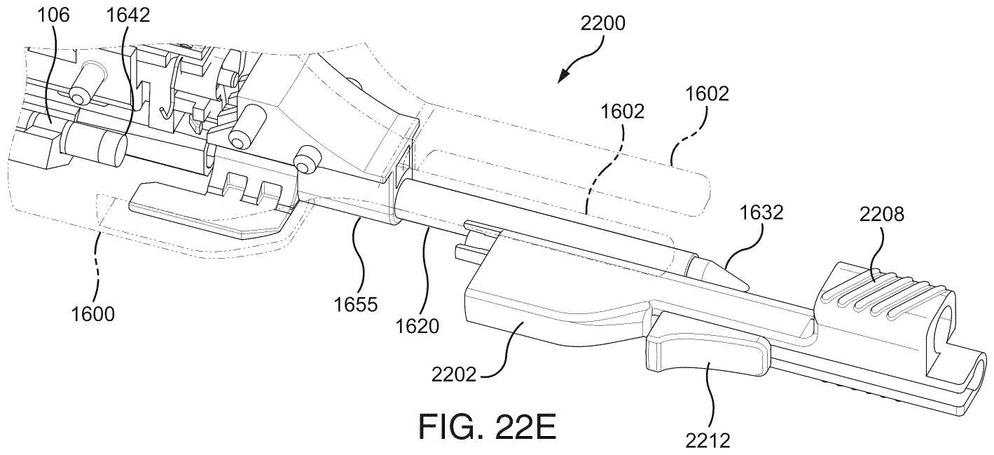

FIG. 22A illustrates a loading tool 2200 embodying aspects of the present disclosure. FIGS. 22B-22E illustrate steps of a loading process for using the loading tool 2200 to load a device 106 into a cannula 1442 or 1642 of an insertion tool 1400 or 1600 embodying aspects of the present disclosure. In some aspects, the device 106 may be loaded into the insertion tool 1400 or 1600 before the insertion tool 1400 or 1600 is placed in the package 1500 or 1700. In some alternative aspects, the device 106 may be loaded into the insertion tool 1400 or 1600 after the package 1500 or 1700 is opened (e.g., after opening the package in step 1902 of the process 1900 shown in FIG. 19 and before the step 1904 of using the tubing 1508 to provide hydration fluid to the passage of the cannula 1442 or 1642).

In some aspects, as shown in FIGS. 22A-22E, the loading tool 2200 may include a body 2202, a passage 2204, a slider 2206, and/or a clip 2208. In some aspects, the slider 2206 may include an opening 2210 for receiving the device 106 and/or an actuator 2212. In some aspects, the slider 2206 may be configured to move within the passage 2204 of the body 2202 of the loading tool 2200 between a forward position (e.g., as shown in FIGS. 22B, 22D, and 22E) and a rearward position (e.g., as shown in FIGS. 22A and 22C). In some aspects, the body 2202 may include a slot 2214 configured to allow movement of the actuator 2210 when the slider 2206 moves between the forward position and the rearward position. In some aspects, as shown in FIG. 22D, the clip 2208 may be configured to attach the loading tool 2200 to the insertion device 1400 or 1600 (e.g., to a guide prong 1402 or 1602 of the insertion tool 1400 or 1600) for loading the device 106 into the cannula 1442 or 1642 of the insertion tool 1400 or 1600.

In some aspects, as shown in FIG. 22B, the loading process may include a first step of loading a device 106 into a loading tool 2200 (e.g., into the passage 2204 of the loading tool 2200). In some aspects, the first step of loading the device 106 into the loading tool 2200 may be performed with the slider 2206 in the forward position. In some aspects, when loaded into the passage 2204 of the loading tool 2200, the device 106 may be received into the opening 2210 of the slider 2206. In some aspects, the actuator 2212 may be configured to keep the slider 2206 in the forward position during loading of the device 106 into the loading tool 2200 (e.g., so that the device 106 may be received into the opening 2210 of the slider 2206).

In some aspects, as shown in FIG. 22C, the loading process may include a second step of moving the slider 2206 from the forward position to the rearward position, which may pull the device 106 into the passage 2204 of the insertion tool 1400 or 1600. In some aspects, the body 2202 may be configured to keep the slider 2206 in the rearward position until the device 106 is loaded into the insertion tool 1400 or 1600.

In some aspects, as shown in FIG. 22D, the loading process may include a third step of moving the slider 2206 from the rearward position to the forward position, which may push the device 106 forward, out of the passage 2204 of the loading tool 2200, and into the cannula 1442 or 1642 of the insertion tool 1400 or 1600. In some aspects, the passage 2204 of the loading tool 2200 may be aligned with and/or inserted into the loading port 1443 or 1643 of the insertion tool 1400 or 1600. In some aspects, as shown in FIG. 22D, the clip 2208 of the loading tool 2200 may be attached to the insertion tool 1400 or 1600 (e.g., attached to a guide prong 1402 or 1602 of the insertion tool 1400 or 1600) when the slider 2206 is moved forward to push the device 106 forward from the passage 2204 of the loading tool 2200 and into the cannula 1442 or 1642 of the insertion tool 1400 or 1600.

In some aspects, as shown in FIG. 22E, the loading process may include a fourth step of removing the loading tool 2200 from the insertion tool 1400 or 1600 and leaving the device 106 in the cannula 1442 or 1642 of the insertion tool 1400 or 1600. In some aspects, the material (e.g., polytetrafluoroethylene (PTFE)) inside the cannula 1442 or 1642 may be such that the cannula 1442 or 1642 retains the device 106 as the loading tool 2200 is removed from the insertion tool 1400 or 1600.

While the subject matter of this disclosure has been described and shown in considerable detail with reference to certain illustrative aspects, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A method for creating a subcutaneous pocket below a skin surface and implanting a device in the subcutaneous pocket, the method comprising:
using a dissector of an insertion tool with the dissector in an extended position in which a blunt tip of the dissector protrudes out of a distal end of a tunneling tube to create the subcutaneous pocket, wherein the tunneling tube defines a passage opening into a cavity of the handle;
moving the dissector from the extended position to a retracted position in which the blunt tip is disposed in the cavity of the handle;
rotating the dissector from an operating position disposed on a longitudinal axis of the tunneling tube to an idle position disposed off the longitudinal axis of the tunneling tube;
rotating an inserter of the insertion tool from an idle position disposed off the longitudinal axis of the tunneling tube to an operating position disposed on the longitudinal axis of the tunneling tube, wherein the inserter holds the device in a passage of a cannula of the inserter;
moving the inserter from a retracted position to an extended position in which the cannula is at least partially in the tunneling tube; and
retracting the tunneling tube to release the device out of the tunneling tube and deploy the device in the subcutaneous pocket.

2. The method of claim 1, further comprising:
moving a slider knob of an actuator of the insertion tool in a first direction to move the dissector from the extended position to the retracted position in which the blunt tip is disposed in the cavity of the handle;
moving the slider knob in a second direction to move the inserter from the retracted position to the extended position; and
moving the slider knob in the first direction to retract the tunneling tube.

3. The method of claim 2, further comprising:
using a stopper of the insertion tool to prevent movement of the slider knob while the dissector is used create the subcutaneous pocket; and
removing the stopper of the insertion tool;
wherein moving the slider knob in the first direction to move the dissector from the extended position to the retracted position in which the blunt tip is disposed in the cavity of the handle occurs after removal of the stopper.

4. The method of claim 1, further comprising using tubing connected to the cannula to provide hydration fluid to the passage of the cannula.

5. The method of claim 4, wherein the tubing connects a hydration fluid container and the cannula, and the method further comprises using the hydration fluid container to provide the hydration fluid to the passage of the cannula via the tubing.

6. The method of claim 1, wherein rotating the dissector and rotating the inserter comprise using a swing rotatably coupled to the dissector and the inserter.

7. The method of claim 1, wherein moving the inserter from the retracted position to the extended position comprises moving the inserter from the retracted position to the extended position while the inserter is in the operating position.

8. The method of claim 1, wherein the dissector comprises a first rod configured to move along the cavity of the handle and the passage of the tunneling tube, and the blunt tip is coupled to the first rod such that the first rod is configured to move the blunt tip along the cavity of the handle and the passage of the tunneling tube between the retracted and extended positions.

9. The method of claim 1, wherein the insertor comprises a second rod configured to move along the cavity of the handle and the passage of the tunneling tube, and the cannula comprises a first end configured to hold and release the device and a second end coupled to the second rod such that the second rod is configured to move the cannula along the cavity of the handle and the passage of the tunneling tube between the retracted and extended positions.

10. The method of claim 9, wherein the cannula retracts with tunneling tube, and the method further comprises using the second rod to force the device at least partially out of the cannula and the tunneling tube.

11. An insertion tool for creating a subcutaneous pocket below a skin surface and implanting a device in the subcutaneous pocket, the insertion tool comprising:
a handle defining a cavity therein;
a tunneling tube defining a passage opening into the cavity of the handle;
a dissector comprising a blunt tip and configured to:
(i) create the subcutaneous pocket;
(ii) move from an extended position, wherein the blunt tip protrudes out of a distal end of the tunneling tube, to a retracted position, wherein the blunt tip is disposed in the cavity of the handle; and
(iii) rotate from an operating position disposed on a longitudinal axis of the tunneling tube to an idle position disposed off the longitudinal axis of the tunneling tube; and
an insertor comprising a cannula and configured to:
(i) hold the device in a passage of the cannula;
(ii) rotate from an idle position disposed off the longitudinal axis of the tunneling tube to an operating position disposed on the longitudinal axis of the tunneling tube; and
(iii) move from a retracted position to an extended position in which the cannula is at least partially within the tunneling tube;
wherein the tunneling tube is configured to retract to release the device out of the tunneling tube and deploy the device in the subcutaneous pocket.

12. The insertion tool of claim 11, wherein the actuator comprises a slider knob configured to cause the inserter and the dissector to move between the retracted and extended positions.

13. The insertion tool of claim 11, further comprising a removable stopper configured to prevent movement of the slider knob.

14. The insertion tool of claim 11, further comprising tubing connected to the cannula and configured to provide hydration fluid to the passage of the cannula.

15. The insertion tool of claim 11, wherein the handle comprises a swing rotatably coupled to the dissector and the inserter.

16. The insertion tool of claim 11, wherein the inserter is configured to move from the retracted position to the extended position when the inserter is set at the operating position.

17. The insertion tool of claim 11, wherein the dissector comprises a first rod configured to move along the cavity of the handle and the passage of the tunneling tube, and the blunt tip is coupled to the first rod such that the first rod is configured to move the blunt tip along the cavity of the handle and the passage of the tunneling tube between the retracted and extended positions.

18. The insertion tool of claim 11, wherein the insertor comprises a second rod configured to move along the cavity of the handle and the passage of the tunneling tube, and the cannula comprises a first end configured to hold and release the device and a second end coupled to the second rod such that the second rod is configured to move the cannula along the cavity of the handle and the passage of the tunneling tube between the retracted and extended positions.

19. The insertion tool of claim 18, wherein the cannula is configured to retract with tunneling tube, and the second rod is configured to force the device at least partially out of the cannula and the tunneling tube.

20. A package comprising:
an insertion tool;
a hydration fluid container; and
tubing connecting the hydration fluid container to the insertion tool and configured to provide hydration fluid from the hydration fluid container to the insertion tool.

21. A package comprising:
a base including a cavity, wherein the base is configured to support a product at least partially within the cavity; and
a stand configured to (i) fit at least partially within the cavity of the base, (ii) when the stand is at least partially within the cavity of the base, hold the product at least partially within the cavity of the base between the stand and a bottom surface of the cavity of the base, (iii) attach to an exterior bottom surface of the base, and (iv) when the stand is attached to the exterior bottom surface of the base, define an angle of tilt of the product supported within the cavity of the base.

22. A method of loading a device into an insertion tool, the method comprising:
moving a slider of a loading tool from a forward position to a rearward position so that the slider pulls the device into a passage of the insertion tool;
moving the slider from the rearward position to the forward position so that the slider pushes the device forward, out of the passage of the loading tool, and into a cannula of the insertion tool; and
removing the loading tool from the insertion tool and leaving the device in the cannula of the insertion tool.
